(19) United States
(12) Patent Application Publication
(10) Pub. No.: US 2021/0198366 A1
(43) Pub. Date: Jul. 1, 2021

(54) B7-H7-BINDING AGENTS AND METHODS OF USE THEREOF

(71) Applicant: NGM Biopharmaceuticals, Inc., South San Francisco, CA (US)

(72) Inventors: Jer-Yuan Hsu, San Bruno, CA (US); Suzanne Christine Crawley, Brisbane, CA (US)

(21) Appl. No.: 17/269,635

(22) PCT Filed: Aug. 20, 2019
(86) PCT No.: PCT/US19/47250

§ 371 Date: Feb. 19, 2021

Related U.S. Application Data
(60) Provisional application No. 62/720,708, filed on Aug. 21, 2018.

(51) Int. Cl. C07K 16/28; A61K 45/06; A61P 35/00

(52) U.S. Cl. CPC C07K 16/2827; A61K 2039/505; A61P 35/00; A61K 45/06

(57) ABSTRACT

The present disclosure provides binding agents, such as antibodies, that specifically bind B7-H7, including human B7-H7, as well as compositions comprising the binding agents and methods of their use. The disclosure also provides related polynucleotides and vectors encoding the binding agents and cells comprising the binding agents.

### CROSS-REFERENCE TO RELATED APPLICATIONS

This application claims the priority benefit of U.S. Provisional Application No. 62/720,708, filed Aug. 21, 2018, the contents of which are incorporated in their entirety herein.

### SEQUENCE LISTING

The present specification is being filed with a computer readable form (CRF) copy of the Sequence Listing. The CRF entitled 47702-0013WO1_SL.txt, which was created on Aug. 20, 2019 and is 110,754 bytes in size, is identical to the paper copy of the Sequence Listing and is incorporated herein by reference in its entirety.

### FIELD OF THE INVENTION

The present disclosure generally relates to agents that bind B7-H7, particularly antibodies that bind human B7-H7, as well as compositions comprising the B7-H7 binding agents and therapeutic methods of using the agents and compositions.

### BACKGROUND

The basis for immunotherapy is the manipulation and/or modulation of the immune system, including both innate immune responses and adaptive immune responses. The general aim of immunotherapy is to treat diseases by controlling the immune response to a “foreign agent”, for example a pathogen or a tumor cell. However, in some instances, immunotherapy is used to treat autoimmune diseases which may arise from an abnormal immune response against proteins, molecules, and/or tissues normally present in the body. Immunotherapy may include agents and methods to induce or enhance specific immune responses or to inhibit or reduce specific immune responses.

The immune system is a highly complex system made up of a great number of cell types, including but not limited to, T-cells, B-cells, natural killer cells, antigen-presenting cells, dendritic cells, monocytes, and macrophages. These cells possess complex and subtle systems for controlling their interactions and responses. The cells utilize both activating and inhibitory mechanisms and feedback loops to keep responses in check and not allow negative consequences of an uncontrolled immune response (e.g., autoimmune diseases).

The concept of cancer immunosurveillance is based on the theory that the immune system can recognize tumor cells, mount an immune response, and suppress the development and/or progression of a tumor. However, it is clear that many cancerous cells have developed mechanisms to evade the immune system which can allow for uninhibited growth of tumors. Cancer/tumor immunotherapy focuses on the development of new and novel agents that can activate and/or boost the immune system to achieve a more effective attack against tumor cells resulting in increased killing of tumor cells and/or inhibition of tumor growth.

### BRIEF SUMMARY

B7-H7 is a member of the B7 family of proteins involved in immune regulation. The B7 family currently includes B7-1 (CD80), B7-2 (CD86), B7-H1 (PD-L1), B7-DC (PD-L2), B7-H2 (ICOS-L), B7-H3, B7-H4 (VTCN1), B7-H5 (VISTA), B7-H6, and B7-H7 (HHLA2). The B7 family proteins function as co-stimulatory or co-inhibitory molecules, generally in collaboration with receptors of the CD28 family to modulate immune responses.

The present disclosure provides agents that bind B7-H7. The agents include, but are not limited to, polypeptides such as antibodies that specifically bind B7-H7. The agents may be referred to herein as “B7-H7-binding agents”. The disclosure provides methods of using a B7-H7-binding agent. In some embodiments, a B7-H7-binding agent induces, activates, promotes, increases, enhances, or prolongs an immune response. In some embodiments, a B7-H7-binding agent increases natural killer cell activity. In some embodiments, a B7-H7-binding agent inhibits growth of tumor cells. In some embodiments, a B7-H7-binding agent is used in a method of treating cancer. In some embodiments, a B7-H7-binding agent is used in combination therapy. In some embodiments, a B7-H7-binding agent is used in combination with at least one additional therapeutic agent.

The disclosure also provides compositions comprising the B7-H7-binding agents described herein. In some embodiments, the disclosure provides pharmaceutical compositions comprising the B7-H7-binding agents described herein. Polynucleotides and/or vectors encoding the B7-H7-binding agents and methods of making the agents are also provided. Cells comprising or producing the B7-H7-binding agents described herein are provided. Cells comprising the polynucleotides and/or the vectors described herein are also provided.

In one aspect, the present disclosure provides agents that bind B7-H7. In some embodiments, the agent binds human B7-H7. In some embodiments, an agent binds SEQ ID NO:2. In some embodiments, the agent binds cynomolgus monkey (“cyno”) B7-H7. In some embodiments, an agent binds SEQ ID NO:8. In some embodiments, the agent binds human B7-H7 and cyno B7-H7. In some embodiments, the agent is an antibody. In some embodiments, the agent is an antibody that binds human B7-H7. In some embodiments, the agent is an antibody that binds cyno B7-H7. In some embodiments, the agent is an antibody that binds human B7-H7 and cyno B7-H7.

In some embodiments, a B7-H7-binding agent (e.g., an antibody) binds the extracellular domain of B7-H7. In some embodiments, a B7-H7-binding agent binds the extracellular domain of human B7-H7. In some embodiments, a B7-H7-binding agent binds SEQ ID NO:3. In some embodiments, a B7-H7-binding agent binds the extracellular domain of cyno B7-H7. In some embodiments, a B7-H7-binding agent binds SEQ ID NO:9. In some embodiments, a B7-H7-binding agent binds an Ig-like domain within B7-H7. In some embodiments, a B7-H7-binding agent binds an Ig-like domain within the extracellular domain of B7-H7. In some embodiments, a B7-H7-binding agent binds an IgV-type domain of B7-H7. In some embodiments, a B7-H7-binding agent binds the IgV-type domain 1 of B7-H7. In some embodiments, a B7-H7-binding agent binds the IgV-type domain 1 of human B7-H7. In some embodiments, a B7-H7-binding agent binds SEQ ID NO:4. In some embodiments, a B7-H7-binding agent binds the IgV-type domain 1 of cyno B7-H7. In some embodiments, a B7-H7-binding agent binds SEQ ID NO: 10. In some embodiments, a B7-H7-binding agent binds the IgV-type domain 2 of B7-H7. In some embodiments, a B7-H7-binding agent binds the IgV-type domain 2 of human B7-H7. In some embodiments, a B7-H7-binding agent binds SEQ ID NO: 6. In some embodiments, a B7-H7-binding agent binds the IgV-type domain 2 of cyno B7-H7. In some embodiments, a B7-H7-binding agent binds SEQ ID NO: 12. In some embodiments, a B7-H7-binding agent binds an IgC-type domain of B7-H7. In some embodiments, a B7-H7-binding agent binds the IgC1 type domain of B7-H7. In some embodiments, a B7-H7-binding agent binds the IgC1 type domain of human B7-H7. In some embodiments, a B7-H7-binding agent binds SEQ ID NO:5. In some embodiments, a B7-H7-binding agent binds the IgC1 type domain of cyno B7-H7. In some embodiments, a B7-H7-binding agent binds SEQ ID NO: 11.

In one aspect, the disclosure provides an agent (e.g., an antibody) that binds B7-H7 and that has at least one (e.g., 1, 2, 3, 4, 5, 6, 7, 8, 9, 10, 11, or 12) of the following properties: (1) the agent binds human B7-H7; (2) the agent binds cyno B7-H7; (3) binds both human B7-H7 and cyno B7-H7; (4) the agent binds the extracellular domain of human B7-H7; (5) the agent binds the extracellular domain of human cyno B7-H7; (6) the agent binds the extracellular domain of human and cyno B7-H7; (7) the agent inhibits binding of B7-H7 to CD28H; (8) the agent inhibits binding of B7-H7 to KIR3DL3; (9) the agent induces NK cell cytotoxicity; (10) the agent enhances production of IFN-gamma; (11) the agent enhances production of TNF-alpha; and (12) the agent enhances production of IFN-gamma and TNF-alpha. In some embodiments, the binding molecule comprises the six CDRs of any of the anti-B7-H7 antibodies described herein. In some embodiments, the binding molecule comprises an amino acid sequence that is at least 75%, 80%, 85%, 90%, 95%, 96%, 97%, 98%, or 99% identical to the VH and/or VL sequences of any of the anti-B7-H7 antibodies described herein.

In one aspect, the disclosure provides an agent (e.g., an antibody) that specifically binds B7-H7, wherein the agent comprises a heavy chain variable region and a light chain variable region, wherein the heavy chain variable region comprises a heavy chain variable region CDR1, a heavy chain variable region CDR2, and a heavy chain variable region CDR3 of the heavy chain variable regions of the anti-B7-H7 antibodies disclosed herein; and wherein the light chain variable region comprises a light chain variable region CDR1, a light chain variable region CDR2, and a light chain variable region CDR3 of the light chain variable regions of the anti-B7-H7 antibodies disclosed herein. The CDRs can be based on any definition known to those of skill in the art and/or as defined herein.

In some embodiments, an agent (e.g., an antibody) that specifically binds B7-H7 comprises: (a) a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15); and/or (b) a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, an agent that specifically binds B7-H7 comprises a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15). In some embodiments, an agent that specifically binds B7-H7 comprises a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, an agent that specifically binds B7-H7 comprises: (a) a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15); and (b) a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, an agent that specifically binds B7-H7 comprises: (a) a heavy chain variable region comprising a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15); and (b) a light chain variable region comprising a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18).

In some embodiments, an agent (e.g., an antibody) that specifically binds B7-H7 comprises: (a) a heavy chain framework region (FR) 1, a heavy chain FR2, a heavy chain FR3, and a heavy chain FR4; and/or (b) a light chain FR1, a light chain FR2, a light chain FR3, and a light chain FR4. In some embodiments, an agent that specifically binds B7-H7 comprises a heavy chain FR1, a heavy chain FR2, a heavy chain FR3, and a heavy chain FR4. In some embodiments, an agent that specifically binds B7-H7 comprises a light chain FR1, a light chain FR2, a light chain FR3, and a light chain FR4. In some embodiments, an agent that specifically binds B7-H7 comprises: (a) a heavy chain FR1, a heavy chain FR2, a heavy chain FR3, and a heavy chain FR4; and (b) a light chain FR1, a light chain FR2, a light chain FR3, and a light chain FR4.

In some embodiments, an agent (e.g., an antibody) that specifically binds B7-H7 comprises: (a) a heavy chain variable region having at least 90% sequence identity to SEQ ID NO: 19 or SEQ ID NO:21; and/or (b) a light chain variable region having at least 90% sequence identity to SEQ ID NO:20 or SEQ ID NO:22. In some embodiments, an agent that specifically binds B7-H7 comprises a heavy chain variable region having at least 95% sequence identity to SEQ ID NO: 19 and a light chain variable region having at least 95% sequence identity to SEQ ID NO:20. In some embodiments, an agent that specifically binds B7-H7 comprises a heavy chain variable region having at least 95% sequence identity to SEQ ID NO:21 and a light chain variable region having at least 95% sequence identity to SEQ ID NO:22. In some embodiments, an agent that specifically binds B7-H7 comprises a heavy chain variable region comprising SEQ ID NO: 19 and a light chain variable region comprising SEQ ID NO:20. In some embodiments, an agent that specifically binds B7-H7 comprises a heavy chain variable region comprising SEQ ID NO:21 and a light chain variable region comprising SEQ ID NO:22. In some embodiments, the agent that specifically binds B7-H7 is an antibody.

In some embodiments, an agent (e.g., an antibody) that specifically binds B7-H7 comprises the heavy chain variable region CDR1, CDR2, and CDR3, and/or the light chain variable region CDR1, CDR2, and CDR3 from: (a) the antibody designated 2D7 that comprises a heavy chain variable region comprising SEQ ID NO: 19 and a light chain variable region comprising SEQ ID NO:20; or (b) the antibody designated Hz2D7 that comprises a heavy chain variable region comprising SEQ ID NO:21 and a light chain variable region comprising SEQ ID NO:22. In some embodiments, an agent (e.g., an antibody) that specifically binds B7-H7 comprises the CDR1, CDR2, and CDR3 from a heavy chain variable region comprising SEQ ID NO: 19 and the CDR1, CDR2, and CDR3 from a light chain variable region comprising SEQ ID NO:20. In some embodiments, an agent (e.g., an antibody) that specifically binds B7-H7 comprises the CDR1, CDR2, and CDR3 from a heavy chain variable region comprising SEQ ID NO:21 and the CDR1, CDR2, and CDR3 from a light chain variable region comprising SEQ ID NO:22.

In some embodiments, an agent (e.g., an antibody) that specifically binds B7-H7 comprises the CDR1, CDR2, and CDR3 from a heavy chain variable region comprising SEQ ID NO: 19 and the CDR1, CDR2, and CDR3 from a light chain variable region comprising SEQ ID NO:20. In some embodiments, an agent (e.g., an antibody) that specifically binds B7-H7 comprises the CDR1, CDR2, and CDR3 from a heavy chain variable region comprising SEQ ID NO:21 and the CDR1, CDR2, and CDR3 from a light chain variable region comprising SEQ ID NO:22.

In some embodiments, a B7-H7-binding agent is an antibody that comprises a heavy chain of SEQ ID NO:24 and/or a light chain of SEQ ID NO:26. In some embodiments, a B7-H7-binding agent is an antibody that comprises a heavy chain of SEQ ID NO:24 and a light chain of SEQ ID NO:26.

In some embodiments, an agent (e.g., an antibody) that specifically binds B7-H7 comprises: (a) a heavy chain variable region CDR1 comprising GYSFTDYIIV (SEQ ID NO:27), a heavy chain variable region CDR2 comprising KINPYYGTTTYNLRFED (SEQ ID NO:28), and a heavy chain variable region CDR3 comprising WDYVSTLFAMDY (SEQ ID NO:29), and/or (b) a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, an agent (e.g., an antibody) that specifically binds B7-H7 comprises: (a) a heavy chain variable region CDR1 comprising GYTFTEYTMH (SEQ ID NO: 13), a heavy chain variable region CDR2 comprising GINPNNGGAPYNQKFKG (SEQ ID NO:34), and a heavy chain variable region CDR3 comprising GGYYFDY (SEQ ID NO: 15), and/or (b) a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, an agent (e.g., an antibody) that specifically binds B7-H7 comprises: (a) a heavy chain variable region CDR1 comprising GYTFSSYSMH (SEQ ID NO:38), a heavy chain variable region CDR2 comprising TIYPGNENTSYNQKFKG (SEQ ID NO:39), and a heavy chain variable region CDR3 comprising GGYYFDY (SEQ ID NO: 15), and/or a light chain variable region CDR1 comprising KASQDVSTAVA (SEQ ID NO:35), a light chain variable region CDR2 comprising WASTRHT (SEQ ID NO:30), and a light chain variable region CDR3 comprising (QQHFDIPYW (SEQ ID NO:40). In some embodiments, an agent (e.g., an antibody) that specifically binds B7-H7 comprises: (a) a heavy chain variable region CDR1 comprising GYTFTTYTMH (SEQ ID NO:43), a heavy chain variable region CDR2 comprising YINPSRGYSDYSQKFQG (SEQ ID NO:44), and a heavy chain variable region CDR3 comprising GGYDFDY (SEQ ID NO:45), and/or a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, an agent (e.g., an antibody) that specifically binds B7-H7 comprises: (a) a heavy chain variable region CDR1 comprising GFSLTGYGVN (SEQ ID NO:49), a heavy chain variable region CDR2 comprising VIWGDGSTDYNSVLKS (SEQ ID NO:50), and a heavy chain variable region CDR3 comprising EATEYLYWYFDV (SEQ ID NO:51), and/or a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, an agent (e.g., an antibody) that specifically binds B7-H7 comprises: (a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SVYDAPWLAH (SEQ ID NO:59); and/or (b) a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, an agent (e.g., an antibody) that specifically binds B7-H7 comprises: (a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SFYDAPYLTY (SEQ ID NO:65); and/or (b) a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, an agent (e.g., an antibody) that specifically binds B7-H7 comprises: (a) a heavy chain variable region CDR1 comprising GYTFTSFWIH (SEQ ID NO:69), a heavy chain variable region CDR2 comprising YIIPNTDYTEYNQKFKD (SEQ ID NO:70), and a heavy chain variable region CDR3 comprising GLRGAYYFDY (SEQ ID NO:71); and/or (b) a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74).

In another aspect of the disclosure, provided herein is an antibody that competes for binding to B7-H7 with any of the B7-H7-binding agents described herein. In some embodiments, provided herein is an antibody that competes for binding to B7-H7 with a reference antibody, wherein the reference antibody comprises: (a) a heavy chain variable region CDR1 comprising GYTFTEYTMH (SEQ ID NO: 13); a heavy chain variable region CDR2 comprising GINPNNYGAPYNQKFKG (SEQ ID NO: 14), and a heavy chain variable region CDR3 comprising GGYYFDY (SEQ ID NO: 15); and (b) a light chain variable region CDR1 comprising KASQDVGTAVA (SEQ ID NO: 16); a light chain variable region CDR2 comprising WAFTRHT (SEQ ID NO: 17); and a light chain variable region CDR3 comprising QQHYDTPFT (SEQ ID NO: 18).

In some embodiments, provided herein is an antibody that competes for binding to B7-H7 with a reference antibody, wherein the reference antibody comprises a heavy chain variable region comprising SEQ ID NO: 19 or SEQ ID NO:21; and a light chain variable region comprising SEQ ID NO:20 or SEQ ID NO:22. In some embodiments, a reference antibody comprises a heavy chain variable region comprising SEQ ID NO: 19 and a light chain variable region comprising SEQ ID NO:20. In some embodiments, a reference antibody comprises a heavy chain variable region comprising SEQ ID NO:21 and a light chain variable region comprising SEQ ID NO:22.

In some embodiments, provided herein is a B7-H7-binding agent that binds the same epitope on B7-H7 as an antibody described herein. In some embodiments, provided herein is a B7-H7-binding agent that binds an epitope on B7-H7 that overlaps with the epitope on B7-H7 bound by an antibody described herein. In some embodiments, provided herein is a B7-H7-binding agent that binds the same epitope as an antibody comprising the heavy chain variable region CDR1, CDR2, and CDR3 and the light chain variable region CDR1, CDR2, and CDR3 of antibody 2D7 or Hz2D7. In some embodiments, provided herein is a B7-H7-binding agent that binds an epitope that overlaps with the epitope bound by an antibody comprising the heavy chain variable region CDR1, CDR2, and CDR3 and the light chain variable region CDR1, CDR2, and CDR3 of antibody 2D7 or Hz2D7. In some embodiments, provided herein is a B7-H7-binding agent that binds the same epitope as an antibody comprising the heavy chain variable region and the light chain variable region from antibody 2D7 or Hz2D7. In some embodiments, provided herein is a B7-H7-binding agent that binds an epitope that overlaps with the epitope bound by an antibody comprising the heavy chain variable region and the light chain variable region from antibody 2D7 or Hz2D7.

In some embodiments, provided herein is a B7-H7-binding agent that competes for binding to B7-H7 with an antibody comprising a heavy chain having the amino acid sequence of SEQ ID NO:24 and a light chain having the amino acid sequence of SEQ ID NO:26.

In some embodiments, provided herein is an antibody that competes for binding to B7-H7 with a reference antibody, wherein the reference antibody comprises: (a) a heavy chain variable region CDR1 comprising GYSFTGYNMN (SEQ ID NO:57); a heavy chain variable region CDR2 comprising NIDPYSGGSTYNQKFKG (SEQ ID NO:58), and a heavy chain variable region CDR3 comprising SVYDAPWLAH (SEQ ID NO:59); and (b) a light chain variable region CDR1 comprising RASENIYIYLA (SEQ ID NO:60); a light chain variable region CDR2 comprising NAKTLAE (SEQ ID NO:61); and a light chain variable region CDR3 comprising QHHYGTPPT (SEQ ID NO:62).

In some embodiments, provided herein is an antibody that competes for binding to B7-H7 with a reference antibody, wherein the reference antibody comprises a heavy chain variable region comprising SEQ ID NO:63 and a light chain variable region comprising SEQ ID NO:64.

In some embodiments, provided herein is an antibody that competes for binding to B7-H7 with a reference antibody, wherein the reference antibody comprises: (a) a heavy chain variable region CDR1 comprising GYTFTSFWIH (SEQ ID NO:69); a heavy chain variable region CDR2 comprising YIIPNTDYTEYNQKFKD (SEQ ID NO:70), and a heavy chain variable region CDR3 comprising GLRGAYYFDY (SEQ ID NO:71); and (b) a light chain variable region CDR1 comprising RSSQSVSTSTNGYMH (SEQ ID NO:72); a light chain variable region CDR2 comprising YASNLES (SEQ ID NO:73); and a light chain variable region CDR3 comprising QHSWVLPYT (SEQ ID NO:74).

In some embodiments, provided herein is an antibody that competes for binding to B7-H7 with a reference antibody, wherein the reference antibody comprises a heavy chain variable region comprising SEQ ID NO:75 and a light chain variable region comprising SEQ ID NO:76.

In some embodiments of each of the aforementioned aspects and embodiments, as well as other aspects and embodiments described herein, a B7-H7-binding agent is an antibody. In some embodiments, the antibody is a monoclonal antibody. In some embodiments, the antibody is a humanized antibody. In some embodiments, the antibody is a human antibody. In some embodiments, the antibody is a chimeric antibody. In some embodiments, the antibody is an IgG antibody. In some embodiments, the antibody is an IgG1 antibody, an IgG2 antibody, an IgG3 antibody, or an IgG4 antibody. In some embodiments, the antibody is a bispecific antibody or a multispecific antibody. In some embodiments, the antibody is an antibody fragment. In some embodiments, the antibody or antibody fragment is a Fab, Fab′, F(ab′)₂, Fv, scFv, (SCFV)₂, single chain antibody, dual variable region antibody, single variable region antibody, linear antibody, or a V region antibody. In some embodiments, the antibody is a scFv antibody. In some embodiments, the antibody is a disulfide-linked scFv (dsscFv).

In some embodiments of each of the aforementioned aspects and embodiments, as well as other aspects and embodiments described herein, a B7-H7-binding agent (e.g., an antibody) described herein inhibits binding of B7-H7 to a B7-H7 receptor. In some embodiments, a B7-H7-binding agent inhibits or blocks the interaction between B7-H7 and a B7-H7 receptor. In some embodiments, the B7-H7 receptor is KIR3DL3. In some embodiments, the B7-H7 receptor is CD28H.

In some embodiments of each of the aforementioned aspects and embodiments, as well as other aspects and embodiments described herein, a B7-H7-binding agent (e.g., an antibody) described herein induces and/or increases an immune response. In some embodiments, the immune response is directed to a tumor or tumor cell. In some embodiments, a B7-H7-binding agent described herein increases cell-mediated immunity. In some embodiments, a B7-H7-binding agent described herein increases natural killer (NK) cell activity. In some embodiments, the NK cell activity is directed to a tumor or tumor cells.

In another aspect, the disclosure provides compositions comprising a B7-H7-binding agent (e.g., an antibody) described herein.

In another aspect, the disclosure provides pharmaceutical compositions comprising a B7-H7-binding agent (e.g., an antibody) described herein and a pharmaceutically acceptable carrier.

In some embodiments of each of the aforementioned aspects, as well as other aspects and/or embodiments described elsewhere herein, the B7-H7-binding agent (e.g., an antibody) is isolated. In some embodiments, the B7-H7-binding agent (e.g., an antibody) is substantially pure.

In another aspect, the disclosure provides polynucleotides comprising a polynucleotide that encodes a B7-H7-binding agent described herein. In some embodiments, the polynucleotide is isolated. In some embodiments, a vector comprises a polynucleotide that encodes a B7-H7-binding agent described herein. In some embodiments, an isolated cell comprises a polynucleotide that encodes a B7-H7-binding agent described herein. In some embodiments, an isolated cell comprises a vector comprising a polynucleotide that encodes a B7-H7-binding agent described herein. In some embodiments, the disclosure provides a cell comprising a B7-H7-binding agent described herein. In some embodiments, the disclosure provides a cell producing a B7-H7-binding agent described herein. In some embodiments, a cell produces an anti-B7-H7 antibody described herein. In some embodiments, a cell is a monoclonal cell line. In some embodiments, a cell is a hybridoma.

In another aspect, the disclosure provides methods of making the B7-H7-binding agents (e.g., antibodies) described herein. In some embodiments, a method of making a B7-H7-binding agent comprises (a) culturing a cell comprising one or more polynucleotides or vectors encoding the B7-H7-binding agent, and (b) isolating the binding agent. In some embodiments, a method further comprises formulating the B7-H7-binding agent as a pharmaceutical composition.

In another aspect, the disclosure provides methods of using the B7-H7-binding agents (e.g., an antibody) described herein. In some embodiments, a method comprises using a composition comprising a B7-H7-binding agent (e.g., an antibody) described herein. In some embodiments, a method comprises using a pharmaceutical composition comprising a B7-H7-binding agent (e.g., an antibody) described herein.

In some embodiments, a method of modulating an immune response in a subject comprises administering to the subject a therapeutically effective amount of a B7-H7-binding agent (e.g., an antibody) described herein. In some embodiments, a method of inducing, activating, promoting, increasing, enhancing, or prolonging an immune response in a subject comprises administering to the subject a therapeutically effective amount of a B7-H7-binding agent (e.g., an antibody) described herein. In some embodiments, the immune response is against a tumor or cancer. In some embodiments, a method of increasing NK cell activity in a subject comprises administering to the subject a therapeutically effective amount of a B7-H7-binding agent (e.g., an antibody) described herein. In some embodiments, the increased NK cell activity is directed to a tumor or cancer.

In some embodiments, a method of inhibiting growth of tumor cells comprises contacting the tumor cells with an effective amount of a B7-H7-binding agent (e.g., an antibody) described herein. In some embodiments, a method of inhibiting growth of a tumor in a subject comprises administering to the subject a therapeutically effective amount of a B7-H7-binding agent (e.g., an antibody) described herein.

In some embodiments, a method of treating cancer in a subject comprises administering to the subject a therapeutically effective amount of a B7-H7-binding agent (e.g., an antibody) described herein.

In some embodiments of the methods described herein, a B7-H7-binding agent described herein is administered to a subject as part of a combination therapy. In some embodiments, a method described herein further comprises administering at least one additional therapeutic agent. In some embodiments, the additional therapeutic agent is a chemotherapeutic agent. In some embodiments, the additional therapeutic agent is a biological agent. In some embodiments, the additional therapeutic agent is an antibody. In some embodiments, the additional therapeutic agent is an immunomodulatory agent. In some embodiments, the additional therapeutic agent is an immune checkpoint inhibitor.

Also disclosed is the use of a B7-H7-binding agent (e.g., an antibody) described herein in the manufacture of a medicament. In some embodiments, use of a B7-H7-binding agent in the manufacture of a medicament for inducing, activating, promoting, increasing, enhancing, or prolonging an immune response is disclosed. In some embodiments, use of a B7-H7-binding agent in the manufacture of a medicament for increasing NK cell activity is disclosed. In some embodiments, use of a B7-H7-binding agent in the manufacture of a medicament for inhibiting growth of a tumor or tumor cells is disclosed. In some embodiments, use of a B7-H7-binding agent in the manufacture of a medicament for treating cancer is disclosed.

In some embodiments of each of the aforementioned aspects and embodiments, as well as other aspects and embodiments described herein, the subject is human.

Where aspects or embodiments of the disclosure are described in terms of a Markush group or other grouping of alternatives, the present disclosure encompasses not only the entire group listed as a whole, but also each member of the group individually and all possible subgroups of the main group, and also the main group absent one or more of the group members. The present disclosure also envisages the explicit exclusion of one or more of any of the group members in the claimed disclosure.

### BRIEF DESCRIPTION OF THE FIGURES

FIG. 1. Diagram of mRNA expression levels of B7-H7 in a number of human cancers and normal tissues based on information for TCGA database.

FIG. 2. Binding of soluble B7-H7 to KIR3DL3 expressed on the surface of cells and binding of soluble KIR3DL3 to B7-H7 expressed on the surface of cells.

FIG. 3. Natural killer cell cytotoxicity assay.

FIG. 4. Blockade of NK suppression by exemplary anti-B7-H7 antibodies.

FIG. 5. Effect of anti-B7-H7 antibodies on IFN-gamma and TNF-alpha secretion in tetanus toxoid recall assay.

FIG. 6. Pharmacokinetic analysis of exemplary anti-B7-H7 antibodies in mice.

FIG. 7. Inhibition of tumor growth in a mouse model.

FIG. 8. Pharmacokinetic analysis of an exemplary anti-B7-H7 antibody in monkeys.

### DETAILED DESCRIPTION

The present disclosure provides novel agents, including, but not limited to, polypeptides such as antibodies, that specifically bind B7-H7. The B7-H7-binding agents include, but are not limited to, polypeptides, antibodies, scaffold proteins, and heterodimeric molecules. B7-H7-binding agents include, but are not limited to, polypeptides that (i) block binding of B7-H7 to a B7-H7 receptor and/or (ii) block or inhibit interaction of B7-H7 to a B7-H7 receptor. Related polypeptides, polynucleotides, vectors, cells, compositions comprising the agents, and methods of making the agents are also provided. Methods of using the novel B7-H7-binding agents are also provided.

### I. Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “binding agent” as used herein refers to a molecule that binds a specific antigen or target (e.g., B7-H7). A binding agent may comprise a protein, peptide, nucleic acid, carbohydrate, lipid, or small molecular weight compound. In some embodiments, a binding agent comprises an antibody or an antigen-binding fragment thereof. In some embodiments, a binding agent is an antibody or an antigen-binding fragment thereof. In some embodiments, a binding agent comprises an alternative protein scaffold or artificial scaffold (e.g., a non-immunoglobulin backbone). In some embodiments, a binding agent is a fusion protein comprising an antigen-binding site. In some embodiments, a binding agent is a bispecific or multispecific molecule comprising at least one antigen-binding site.

The term “antibody” as used herein refers to an immunoglobulin molecule that recognizes and binds a target through at least one antigen-binding site. “Antibody” is used herein in the broadest sense and encompasses various antibody structures, including but not limited to, polyclonal antibodies, recombinant antibodies, monoclonal antibodies, chimeric antibodies, humanized antibodies, human antibodies, bispecific antibodies, multispecific antibodies, diabodies, tribodies, tetrabodies, single chain Fv (scFv) antibodies, and antibody fragments as long as they exhibit the desired antigen-binding activity.

The term “intact antibody” or “full-length antibody” refers to an antibody having a structure substantially similar to a native antibody structure. This includes, for example, an antibody comprising two light chains each comprising a variable region and a light chain constant region (CL) and two heavy chains each comprising a variable region and at least heavy chain constant regions CH1, CH2, and CH3. Generally, an intact antibody includes a hinge region (or a portion thereof) between the CH1 and CH2 regions.

The term “antibody fragment” as used herein refers to a molecule other than an intact antibody that comprises a portion of an antibody and generally an antigen-binding site. Examples of antibody fragments include, but are not limited to, Fab, Fab′, F(ab′)2, Fv, single chain antibody molecules (e.g., scFv), sc(Fv)₂, disulfide-linked scFv (dsscFv), diabodies, tribodies, tetrabodies, minibodies, dual variable domain antibodies (DVD), single variable domain antibodies (e.g., camelid antibodies), and multispecific antibodies formed from antibody fragments.

The term “monoclonal antibody” as used herein refers to a substantially homogenous antibody population involved in the highly specific recognition and binding of a single antigenic determinant or epitope. The term “monoclonal antibody” encompasses intact and full-length monoclonal antibodies as well as antibody fragments (e.g., Fab, Fab′, F(ab′)₂, Fv), single chain antibodies (e.g., scFv), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR amino acid residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region amino acid residues of the human heavy chain or light chain variable regions are replaced by corresponding residues from nonhuman antibody. Furthermore, humanized antibodies can comprise amino acid residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc, and/or hinge region), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography may be used to characterize an epitope on a target protein by analyzing the amino acid residue interactions of an antigen/antibody complex.

The term “specifically binds” as used herein refers to an agent (e.g., an antibody) that interacts more frequently, more rapidly, with greater duration, with greater affinity, or with some combination of the above to a particular antigen, epitope, protein, or target molecule than with alternative substances. A binding agent (e.g. antibody) that specifically binds an antigen can be identified, for example, by immunoassays, ELISAs, surface plasmon resonance (SPR), or other techniques known to those of skill in the art. In some embodiments, an agent (e.g., an antibody) that specifically binds an antigen (e.g., human B7-H7) can bind related antigens (e.g., cyno B7-H7). A binding agent that specifically binds an antigen can bind the target antigen at a higher affinity than its affinity for a different antigen. The different antigen can be a related antigen. In some embodiments, a binding agent that specifically binds an antigen can bind the target antigen with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology in a Biacore system as described herein or as known to those of skill in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, (i) the coding region of a nucleotide sequence or (ii) an amino acid sequence.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies do not abrogate the binding of the polypeptide or antibody to the target binding site. Methods of identifying nucleotide and amino acid conservative substitutions that do not eliminate binding are well-known in the art.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like.

The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” or “acceptable pharmaceutical carrier” as used herein refer to an excipient, carrier, or adjuvant that can be administered to a subject, together with at least one therapeutic agent (e.g., an antibody), and that is generally safe, non-toxic, and has no effect on the pharmacological activity of the therapeutic agent. In general, those of skill in the art and the U.S. FDA consider a pharmaceutically acceptable excipient, carrier, or adjuvant to be an inactive ingredient of any formulation.

The term “pharmaceutical formulation” or “pharmaceutical composition” as used herein refers to a preparation that is in such form as to permit the biological activity of the agent (e.g, an antibody) to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent (e.g, an antibody) that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g, an agent other than the binding agents provided herein).

The term “therapeutic effect” as used herein refers to the effect and/or ability of an agent (e.g, an antibody) to reduce and/or ameliorate the severity and/or duration of (i) a disease, disorder, or condition in a subject, and/or (ii) a symptom in a subject. The term also encompasses the ability of an agent to (i) reduce or ameliorate the advancement or progression of a given disease, disorder, or condition, (ii) reduce or ameliorate the recurrence, development, or onset of a given disease, disorder, or condition, and/or (iii) to improve or enhance the prophylactic or therapeutic effect(s) of another agent or therapy (e.g, an agent other than the binding agents provided herein).

The term “treat” or “treatment” or “treating” or “to treat” or “alleviate” or alleviation” or “alleviating” or “to alleviate” as used herein refers to both (1) therapeutic measures that aim to cure, slow down, lessen symptoms of, and/or halt progression of a pathologic condition or disorder and (2) prophylactic or preventative measures that aim to prevent or slow the development of a targeted pathologic condition or disorder. Thus, those in need of treatment include those already with the disorder, those at risk of having/developing the disorder, and those in whom the disorder is to be prevented.

The term “prevent” or “prevention” or “preventing” as used herein refers to the partial or total inhibition of the development, recurrence, onset, or spread of a disease, disorder, or condition, or a symptom thereof in a subject.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

As used herein, reference to “about” or “approximately” a value or parameter includes (and describes) embodiments that are directed to that value or parameter. For example, a description referring to “about X” includes description of “X”.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. B7-H7-Binding Agents

B7-H7 is also known as human endogenous retrovirus-H long terminal repeat associating 2, HHLA2, HERV-H LTR-associating 2, B7 Homolog 7, and B7-H5 (not VISTA). B7-H7 is a type I transmembrane molecule that comprises three Ig-like domains (IgV-IgC-IgV) in its extracellular region. Recently CD28H (TMIGD2) was identified as a receptor for B7-H7 (Janakiram et al., 2015, Clinical Cancer Research, 21:2359-2366). B7-H7 has been shown to be constitutively expressed on human monocytes and induced on B cells after stimulation with IFN-gamma. In addition, studies showed that normal tissues do not express B7-H7, with the exception of placenta, intestines, kidney, gallbladder, and breast. (Janakiram, et al., 2017, Immunological Reviews, 276:26-39). Interestingly, B7-H7 has been found to be expressed and/or over-expressed in a number of human cancers, including but not limited to, liver cancer, pancreatic cancer, colorectal cancer, ovarian cancer, breast cancer, prostate cancer, bone cancer, lung cancer, kidney cancer, and melanoma.

Representative amino acid (aa) sequences for human B7-H7 (UniProtKB No. Q9UM44) and cynomolgus monkey (“cyno”) B7-H7 (NCBI Ref. No. XP 015301968.1) are provided herein as SEQ ID NO: 1 and SEQ ID NO:7, respectively. No ortholog or homolog for B7-H7 has been identified in mice or rats. As used herein, reference to amino acid positions of B7-H7 refer to the numbering of amino acid sequences including the signal sequence. Representative amino acid (aa) sequences for human CD28H (UniProtKB No. Q96BF3) and human KIR3DL3 (UniProtKB No. Q8N743) are provided herein as SEQ ID NO:81 and SEQ ID NO:83, respectively.

The present disclosure provides agents that bind B7-H7. In some embodiments, a B7-H7-binding agent binds a fragment of B7-H7. In some embodiments, a B7-H7-binding agent binds the extracellular domain of B7-H7. In some embodiments, a B7-H7-binding agent binds the extracellular domain of human B7-H7. In some embodiments, a B7-H7-binding agent binds SEQ ID NO:3. In some embodiments, a B7-H7-binding agent binds the extracellular domain of cyno B7-H7. In some embodiments, a B7-H7-binding agent binds SEQ ID NO:9. In some embodiments, a B7-H7-binding agent binds an IgV-type domain of B7-H7. In some embodiments, a B7-H7-binding agent binds the IgV-type domain 1 of B7-H7. In some embodiments, a B7-H7-binding agent binds the IgV-type domain 1 of human B7-H7. In some embodiments, a B7-H7-binding agent binds SEQ ID NO:4. In some embodiments, a B7-H7-binding agent binds the IgV-type domain 1 of cyno B7-H7. In some embodiments, a B7-H7-binding agent binds SEQ ID NO: 10. In some embodiments, a B7-H7-binding agent binds the IgV-type domain 2 of human B7-H7. In some embodiments, a B7-H7-binding agent binds SEQ ID NO:6. In some embodiments, a B7-H7-binding agent binds the IgV-type domain 2 of cyno B7-H7. In some embodiments, a B7-H7-binding agent binds SEQ ID NO: 12. In some embodiments, a B7-H7-binding agent binds an IgC-type domain of B7-H7. In some embodiments, a B7-H7-binding agent binds the IgC1 type domain of B7-H7. In some embodiments, a B7-H7-binding agent binds the IgC1 type domain of human B7-H7. In some embodiments, a B7-H7-binding agent binds SEQ ID NO:5. In some embodiments, a B7-H7-binding agent binds the IgC1 type domain of cyno B7-H7. In some embodiments, a B7-H7-binding agent binds SEQ ID NO: 11.

In some embodiments, a B7-H7-binding agent binds an epitope on B7-H7. In some embodiments, a B7-H7-binding agent binds a linear epitope on B7-H7. In some embodiments, a B7-H7-binding agent binds a conformational epitope on B7-H7. In some embodiments, a B7-H7-binding agent binds human B7-H7. In some embodiments, a B7-H7-binding agent binds SEQ ID NO:2. In some embodiments, a B7-H7-binding agent binds cyno B7-H7. In some embodiments, a B7-H7-binding agent binds SEQ ID NO:8. In some embodiments, a B7-H7-binding agent binds human B7-H7 and cyno B7-H7. In some embodiments, a B7-H7-binding agent binds SEQ ID NO:2 and SEQ ID NO:8. In some embodiments, a B7-H7-binding agent binds within amino acids 23-344 of human B7-H7. In some embodiments, a B7-H7-binding agent binds within amino acids 61-131 of human B7-H7. In some embodiments, a B7-H7-binding agent binds within amino acids 138-222 of human B7-H7. In some embodiments, a B7-H7-binding agent binds within amino acids 235-328 of human B7-H7. In some embodiments, a B7-H7-binding agent binds within amino acids 61-222 of human B7-H7. In some embodiments, a B7-H7-binding agent binds within amino acids 138-328 of human B7-H7.

In some embodiments, a B7-H7-binding agent binds within amino acids 23-344 of SEQ ID NO: 1. In some embodiments, a B7-H7-binding agent binds within amino acids 61-131 of SEQ ID NO: 1. In some embodiments, a B7-H7-binding agent binds within amino acids 138-222 of SEQ ID NO: 1. In some embodiments, a B7-H7-binding agent binds within amino acids 235-328 of SEQ ID NO: 1. In some embodiments, a B7-H7-binding agent binds within amino acids 61-222 of SEQ ID NO: 1. In some embodiments, a B7-H7-binding agent binds within amino acids 138-328 of SEQ ID NO: 1.

In some embodiments, a B7-H7-binding agent binds a polypeptide comprising the amino acid sequence of SEQ ID NO:3. In some embodiments, a B7-H7-binding agent binds a polypeptide comprising the amino acid sequence of SEQ ID NO:4. In some embodiments, a B7-H7-binding agent binds a polypeptide comprising the amino acid sequence of SEQ ID NO:5. In some embodiments, a B7-H7-binding agent binds a polypeptide comprising the amino acid sequence of SEQ ID NO:6. In some embodiments, a B7-H7-binding agent binds a polypeptide comprising the amino acid sequence of SEQ ID NO:9. In some embodiments, a B7-H7-binding agent binds a polypeptide comprising the amino acid sequence of SEQ ID NO: 10. In some embodiments, a B7-H7-binding agent binds a polypeptide comprising the amino acid sequence of SEQ ID NO: 11. In some embodiments, a B7-H7-binding agent binds a polypeptide comprising the amino acid sequence of SEQ ID NO: 12.

In some embodiments, the B7-H7-binding agent binds an epitope comprising amino acids within SEQ ID NO:3. In some embodiments, the B7-H7-binding agent binds an epitope comprising amino acids within SEQ ID NO:4. In some embodiments, the B7-H7-binding agent binds an epitope comprising amino acids within SEQ ID NO:5. In some embodiments, the B7-H7-binding agent binds an epitope comprising amino acids within SEQ ID NO:6. In some embodiments, the B7-H7-binding agent binds an epitope comprising amino acids within SEQ ID NO:9. In some embodiments, the B7-H7-binding agent binds an epitope comprising amino acids within SEQ ID NO: 10. In some embodiments, the B7-H7-binding agent binds an epitope comprising amino acids within SEQ ID NO: 11. In some embodiments, the B7-H7-binding agent binds an epitope comprising amino acids within SEQ ID NO: 12. In some embodiments, the B7-H7-binding agent binds at least one amino acid within SEQ ID NO:3. In some embodiments, the B7-H7-binding agent binds at least one amino acid within SEQ ID NO:4. In some embodiments, the B7-H7-binding agent binds at least one amino acid within SEQ ID NO:5. In some embodiments, the B7-H7-binding agent binds at least one amino acid within SEQ ID NO:6. In some embodiments, the B7-H7-binding agent binds at least one amino acid within SEQ ID NO:9. In some embodiments, the B7-H7-binding agent binds at least one amino acid within SEQ ID NO: 10. In some embodiments, the B7-H7-binding agent binds at least one amino acid within SEQ ID NO: 11. In some embodiments, the B7-H7-binding agent binds at least one amino acid within SEQ ID NO: 12.

In some embodiments, a B7-H7-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In some embodiments, the antibody comprises a kappa light chain constant region. In some embodiments, the antibody comprises a lambda light chain constant region. In some embodiments, the antibody is an antibody fragment comprising an antigen-binding site. In some embodiments, the antibody is a scFv. In some embodiments, the antibody is a disulfide-linked scFv. In some embodiments, the antibody is a sc(Fv)₂. In some embodiments, the antibody is a Fab, Fab′, or F(ab)₂ antibody. In some embodiments, the antibody is a diabody or a nanobody. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody.

In some embodiments, the antibody is isolated. In some embodiments, the antibody is substantially pure.

In some embodiments, a B7-H7-binding agent is a polyclonal antibody. Polyclonal antibodies can be prepared by any method known to those of skill in the art. In some embodiments, polyclonal antibodies are produced by immunizing an animal (e.g., a rabbit, rat, mouse, goat, donkey) with an antigen of interest (e.g., a purified peptide fragment, a recombinant protein, or a fusion protein) using multiple subcutaneous or intraperitoneal injections. In some embodiments, the antigen is conjugated to a carrier such as keyhole limpet hemocyanin (KFH), serum albumin, bovine thyroglobulin, or soybean trypsin inhibitor. The antigen (with or without a carrier protein) is diluted in sterile saline and usually combined with an adjuvant (e.g., Complete or Incomplete Freund's Adjuvant) to form a stable emulsion.

After a period of time, polyclonal antibodies are recovered from the immunized animal (e.g., from blood or ascites). In some embodiments, the polyclonal antibodies are purified from serum or ascites according to standard methods in the art including, but not limited to, affinity chromatography, ion-exchange chromatography, gel electrophoresis, and/or dialysis.

In some embodiments, a B7-H7-binding agent is a monoclonal antibody. Monoclonal antibodies can be prepared by any method known to those of skill in the art. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a cyno protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process.

Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody and their sequence is determined using standard techniques. The isolated polynucleotides encoding the heavy and light chains are then cloned into suitable expression vectors that produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable domains or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, a B7-H7-binding agent is a humanized antibody. Various methods for generating humanized antibodies are known in the art. In some embodiments, a humanized antibody comprises one or more amino acid residues that have been introduced into its sequence from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region are used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody.

In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, (i) a method called “superhumanization” that is described as the direct transfer of CDRs to a human germline framework, (ii) a method termed Human String Content (HSC) that is based on a metric of “antibody humanness”, (iii) methods based on generation of large libraries of humanized variants (including phage, ribosomal, and yeast display libraries), and (iv) methods based on framework region shuffling.

In some embodiments, a B7-H7-binding agent is a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, a B7-H7-binding agent is a bispecific antibody. Bispecific antibodies are capable of recognizing and binding at least two different antigens or epitopes. The different epitopes can either be within the same molecule (e.g., two epitopes on B7-H7) or on different molecules (e.g., one epitope on B7-H7 and one epitope on a different target). In some embodiments, a bispecific antibody has enhanced potency as compared to an individual antibody or to a combination of more than one antibody.

In some embodiments, a bispecific antibody has reduced toxicity as compared to an individual antibody or to a combination of more than one antibody. It is known to those of skill in the art that any therapeutic agent may have unique pharmacokinetics (PK) (e.g, circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g, tissue) in a subject. In some embodiments, a bispecific antibody has the ability to concentrate the actions of two agents to a common target (e.g, a specific cell type). In some embodiments, a bispecific antibody has the ability to target the actions of two agents to more than one biological pathway or function. In some embodiments, a bispecific antibody has the ability to target two different cells and bring them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

Several techniques for making bispecific antibodies are known by those skilled in the art. In some embodiments, the bispecific antibodies comprise heavy chain constant regions with modifications in the amino acids that are part of the interface between the two heavy chains. These modifications are made to enhance heterodimer formation and generally reduce or eliminate homodimer formation. In some embodiments, the bispecific antibodies are generated using a knobs-into-holes (KIH) strategy. In some embodiments, the bispecific antibodies comprise variant hinge regions incapable of forming disulfide linkages between identical heavy chains (e.g, reduce homodimer formation). In some embodiments, the bispecific antibodies comprise heavy chains with changes in amino acids that result in altered electrostatic interactions. In some embodiments, the bispecific antibodies comprise heavy chains with changes in amino acids that result in altered hydrophobic/hydrophilic interactions.

Bispecific antibodies can be intact antibodies or antibody fragments comprising antigen-binding sites.

B7-H7-binding agents with more than two specificities are contemplated. In some embodiments, trispecific or tetraspecific antibodies are generated. B7-H7-binding agents with more than two valencies are contemplated. In some embodiments, trivalent or tetravalent antibodies are generated.

In some embodiments, a B7-H7-binding agent is an antibody that binds B7-H7. In some embodiments, an anti-B7-H7 antibody binds human B7-H7 (e.g., SEQ ID NO:2). In some embodiments, an anti-B7-H7 antibody binds cyno B7-H7 (e.g, SEQ ID NO:8). In some embodiments, an anti-B7-H7 antibody binds human and cyno B7-H7. In some embodiments, an anti-B7-H7 antibody binds a portion or fragment of B7-H7. In some embodiments, an anti-B7-H7 antibody binds the extracellular domain of B7-H7. In some embodiments, an anti-B7-H7 antibody binds the extracellular domain of human B7-H7 (e.g., SEQ ID NO:3). In some embodiments, an anti-B7-H7 antibody binds the extracellular domain of cyno B7-H7 (e.g., SEQ ID NO:9). In some embodiments, an anti-B7-H7 antibody binds within amino acids 23-344 of human B7-H7. In some embodiments, an anti-B7-H7 antibody binds an IgV-type domain of B7-H7. In some embodiments, an anti-B7-H7 antibody binds the IgV-type domain 1 of B7-H7. In some embodiments, an anti-B7-H7 antibody binds the IgV-type domain 1 of human B7-H7 (e.g., SEQ ID NO:4). In some embodiments, an anti-B7-H7 antibody binds the IgV-type domain 1 of cyno B7-H7 (e.g., SEQ ID NO: 10). In some embodiments, an anti-B7-H7 antibody binds within amino acids 61-131 of human B7-H7. In some embodiments, an anti-B7-H7 antibody binds the IgV-type domain 2 of B7-H7. In some embodiments, an anti-B7-H7 antibody binds the IgV-type domain 2 of human B7-H7 (e.g., SEQ ID NO:6). In some embodiments, an anti-B7-H7 antibody binds the IgV-type domain 2 of cyno B7-H7 (SEQ ID NO: 12). In some embodiments, an anti-B7-H7 antibody binds within amino acids 235-328 of human B7-H7. In some embodiments, an anti-B7-H7 antibody binds an IgC-type domain of B7-H7. In some embodiments, a B7-H7-binding agent binds the IgC1 type domain of B7-H7. In some embodiments, a B7-H7-binding agent binds the IgC1 type domain of human B7-H7 (e.g., SEQ ID NO:5). In some embodiments, a B7-H7-binding agent binds the IgC1 type domain of cyno B7-H7 (e.g., SEQ ID NO: 11). In some embodiments, an anti-B7-H7 antibody binds within amino acids 138-222 of human B7-H7. In some embodiments, an anti-B7-H7 antibody binds an epitope within the extracellular domain of B7-H7.

CDRs of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequence and determination of CDRs.

The specific CDR sequences defined herein are generally based on a combination of Kabat and Chothia definitions (Exemplary system). However, it will be understood that reference to a heavy chain variable region CDR or CDRs and/or a light chain variable region CDR or CDRs of a specific antibody will encompass all CDR definitions as known to those of skill in the art.

In some embodiments, an anti-B7-H7 antibody described herein comprises the six CDRs of antibody 2D7, Hz2D7, 2F10, 3H6, 5F7, 16D5, 28F5, 1D3, 5B10, or 5C9 based on the Kabat definition. In some embodiments, an anti-B7-H7 antibody described herein comprises the six CDRs of antibody 2D7, Hz2D7, 2F10, 3H6, 5F7, 16D5, 28F5, 1D3, 5B10, or 5C9 based on the Chothia definition. In some embodiments, an anti-B7-H7 antibody described herein comprises the six CDRs of antibody 2D7, Hz2D7, 2F10, 3H6, 5F7, 16D5, 28F5, 1D3, 5B10, or 5C9 based on the AbM definition. In some embodiments, an anti-B7-H7 antibody described herein comprises the six CDRs of antibody 2D7, Hz2D7, 2F10, 3H6, 5F7, 16D5, 28F5, 1D3, 5B10, or 5C9 based on the IMGT definition. In some embodiments, an anti-B7-H7 antibody described herein comprises the six CDRs of antibody 2D7, Hz2D7, 2F10, 3H6, 5F7, 16D5, 28F5, 1D3, 5B10, or 5C9 based on the contact definition. In some embodiments, an anti-B7-H7 antibody described herein comprises the six CDRs of antibody 2D7, Hz2D7, 2F10, 3H6, 5F7, 16D5, 28F5, 1D3, 5B10, or 5C9 based on the Exemplary definition.

In some embodiments, a B7-H7-binding agent is an anti-B7-H7 antibody that comprises one, two, three, four, five, and/or six CDRs of any one of the antibodies described herein. In some embodiments, an anti-B7-H7 antibody comprises (i) one, two, and/or three heavy chain variable region CDRs from Table 1A, and/or (ii) one, two, and/or three light chain variable region CDRs from Table 1A. In some embodiments, an anti-B7-H7 antibody comprises (i) one, two, and/or three heavy chain variable region CDRs from Table IB, and/or (ii) one, two, and/or three light chain variable region CDRs from Table IB. In some embodiments, an anti-B7-H7 antibody comprises (i) one, two, and/or three heavy chain variable region CDRs from Table 2, and/or (ii) one, two, and/or three light chain variable region CDRs from Table 2. In some embodiments, an anti-B7-H7 antibody comprises (i) one, two, and/or three heavy chain variable region CDRs from Table 3, and/or (ii) one, two, and/or three light chain variable region CDRs from Table 3. In some embodiments, an anti-B7-H7 antibody comprises (i) one, two, and/or three heavy chain variable region CDRs from Table 4, and/or (ii) one, two, and/or three light chain variable region CDRs from Table 4. In some embodiments, an anti-B7-H7 antibody comprises (i) one, two, and/or three heavy chain variable region CDRs from Table 5, and/or (ii) one, two, and/or three light chain variable region CDRs from Table 5. In some embodiments, an anti-B7-H7 antibody comprises (i) one, two, and/or three heavy chain variable region CDRs from Table 6, and/or (ii) one, two, and/or three light chain variable region CDRs from Table 6. In some embodiments, an anti-B7-H7 antibody comprises (i) one, two, and/or three heavy chain variable region CDRs from Table 7, and/or (ii) one, two, and/or three light chain variable region CDRs from Table 7. In some embodiments, an anti-B7-H7 antibody comprises (i) one, two, and/or three heavy chain variable region CDRs from Table 8, and/or (ii) one, two, and/or three light chain variable region CDRs from Table 8. In some embodiments, an anti-B7-H7 antibody comprises (i) one, two, and/or three heavy chain variable region CDRs from Table 9, and/or (ii) one, two, and/or three light chain variable region CDRs from Table 9. In some embodiments, an anti-B7-H7 antibody comprises (i) three heavy chain variable region CDRs from Table 1A, and (ii) three light chain variable region CDRs from Table 1A. In some embodiments, an anti-B7-H7 antibody comprises (i) three heavy chain variable region CDRs from Table IB, and (ii) three light chain variable region CDRs from Table IB. In some embodiments, an anti-B7-H7 antibody comprises (i) three heavy chain variable region CDRs from Table 2, and (ii) three light chain variable region CDRs from Table 2. In some embodiments, an anti-B7-H7 antibody comprises (i) three heavy chain variable region CDRs from Table 3, and (ii) three light chain variable region CDRs from Table 3. In some embodiments, an anti-B7-H7 antibody comprises (i) three heavy chain variable region CDRs from Table 4, and (ii) three light chain variable region CDRs from Table 4. In some embodiments, an anti-B7-H7 antibody comprises (i) three heavy chain variable region CDRs from Table 5, and (ii) three light chain variable region CDRs from Table 5. In some embodiments, an anti-B7-H7 antibody comprises (i) three heavy chain variable region CDRs from Table 6, and (ii) three light chain variable region CDRs from Table 6. In some embodiments, an anti-B7-H7 antibody comprises (i) three heavy chain variable region CDRs from Table 7, and (ii) three light chain variable region CDRs from Table 7. In some embodiments, an anti-B7-H7 antibody comprises (i) three heavy chain variable region CDRs from Table 8, and (ii) three light chain variable region CDRs from Table 8. In some embodiments, an anti-B7-H7 antibody comprises (i) three heavy chain variable region CDRs from Table 9, and (ii) three light chain variable region CDRs from Table 9.

TABLE 1A
Antibody 2D7 Sequences

| | Exem- | | | | |
|---|---|---|---|---|---|
| | plary | Chothia | AbM | Kabat | Contact |
| Heavy | GYTFT | GYTFTEY | GYTFTE | EYTMH | TEYTMH |
| Chain | EYTMH | (SEQ ID | YTMH | (SEQ ID | (SEQ ID |
| Variable | (SEQ ID | NO: 85) | (SEQ ID | NO: 88) | NO: 89) |
| Region | NO: 13) | | NO: 13) | | |
| CDR1 | | | | | |
| Heavy | GINPNNYGA | NPNNYG | GINPN | GINPNN | W1GGINP |
| Chain | PYNQKFKG | (SEQ ID | NYGAP | YGAPYN | NNYGAP |
| Variable | (SEQ ID | NO: 86) | (SEQ ID | QKFKG | (SEQ ID |
| Region | NO: 14) | | NO: 87) | (SEQ ID | NO: 90) |
| CDR2 | | | | NO: 14) | |
| Heavy | GGYYFDY | GGYYFDY | GGYYFDY | GGYYFDY | ASGGYYFD |
| Chain | (SEQ ID | (SEQ ID | (SEQ ID | (SEQ ID | (SEQ ID |
| Variable | NO: 15) | NO: 15) | NO: 15) | NO: 15) | NO: 91) |
| Region | | | | | |
| CDR3 | | | | | |
| Light | KASQDV | KASQDV | KASQDV | KASQDV | GTAVAWY |
| Chain | GTAVA | GTAVA | GTAVA | GTAVA | (SEQ ID |
| Variable | (SEQ ID | (SEQ ID | (SEQ ID | (SEQ ID | NO: 92) |
| Region | NO: 16) | NO: 16) | NO: 16) | NO: 16) | |
| CDR1 | | | | | |
| Light | WAFTRHT | WAFTRHT | WAFTRHT | WAFTRHT | LLISWA |
| Chain | (SEQ ID | (SEQ ID | (SEQ ID | (SEQ ID | FTRH |
| Variable | NO: 17) | NO: 17) | NO: 17) | NO: 17) | (SEQ ID |
| Region | | | | | NO: 93) |
| CDR2 | | | | | |
| Light | QQHYD | QQHYD | QQHYD | QQHYD | QQHY |
| Chain | TPFT | TPFT | TPFT | TPFT | DTPF |
| Variable | (SEQ ID | (SEQ ID | (SEQ ID | (SEQ ID | (SEQ ID |
| Region | NO: 18) | NO: 18) | NO: 18) | NO: 18) | NO: 94) |
| CDR3 | | | | | |

2D7 Heavy chain variable region
(SEQ ID NO: 19)
EVQLQQSGPELVKPGASVKISCKTSGYTFTEYTMHWVKQSHGK
SLEWIGGINPNNYGAPYNQKFKGKATLTVDKSSNTAYMELRSL
TSEDSAIYYCASGGYYFDYWGQGTTLTVSS
2D7 Light chain variable region
(SEQ ID NO: 20)
DIVMTQSHKFMSTSFGDRVSITCKASQDVGTAVAWYQQKLGQS
PKLLISWAFTRHTGVPDRFTGSGSGTDYTLTISSLQAEDLALY
YCQQHYDTPFTFGSGTKLEIK

TABLE 1B
Antibody Hz2D7 Sequences

| | Exem- | | | | |
|---|---|---|---|---|---|
| | plary | Chothia | AbM | Kabat | Contact |
| Heavy | GYTFT | GYTFTEY | GYTFT | EYTMH | TEYTMH |
| Chain | EYTMH | (SEQ ID | EYTMH | (SEQ ID | (SEQ ID |
| Variable | (SEQ ID | NO: 85) | (SEQ ID | NO: 88) | NO: 89) |
| Region | NO: 13) | | NO: 13) | | |
| CDR1 | | | | | |
| Heavy | GINPNN | NPNNYG | GINPN | GINPNN | WMGGIN |
| Chain | YGAPYN | (SEQ ID | NYGAP | YGAPYN | PNNYGA |
| Variable | QKFKG | NO: 86) | (SEQ ID | QKFKG | P |
| Region | (SEQ ID | | NO: 87) | (SEQ ID | (SEQ ID |
| CDR2 | NO: 14) | | | NO: 14) | NO: 5) |
| Heavy | GGYY | GGYYFDY | GGYY | GGYY | ASGGYYFD |
| Chain | FDY | (SEQ ID | FDY | FDY | (SEQ ID |
| Variable | (SEQ ID | NO: 15) | (SEQ ID | (SEQ ID | NO: 91) |
| Region | NO: 15) | | NO: 15) | NO: 15) | |
| CDR3 | | | | | |
| Light | KASQD | KASQDV | KASQDV | KASQDV | GTAVAWY |
| Chain | VGTAVA | GTAVA | GTAVA | GTAVA | (SEQ ID |
| Variable | (SEQ ID | (SEQ ID | (SEQ ID | (SEQ ID | NO: 92) |
| Region | NO: 16) | NO: 16) | NO: 16) | NO: 16) | |
| CDR1 | | | | | |
| Light | WAFT | WAFTRHT | WAFT | WAFT | LLIYWA |
| Chain | RHT | (SEQ ID | RHT | RHT | FTRH |
| Variable | (SEQ ID | NO: 17) | (SEQ ID | (SEQ ID | (SEQ ID |
| Region | NO: 17) | | NO: 17) | NO: 17) | NO: 96) |
| CDR2 | | | | | |
| Light | QQHYD | QQHY | QQHYD | QQHYD | QQHY |
| Chain | TPFT | DTPFT | TPFT | TPFT | DTPF |
| Variable | (SEQ ID | (SEQ ID | (SEQ ID | (SEQ ID | (SEQ ID |
| Region | NO: 18) | NO: 18) | NO: 18) | NO: 18) | NO: 94) |
| CDR3 | | | | | |

Hz2D7 Heavy chain variable region (SEQ ID NO: 21)
QVQLVQSGAEVKKPGASVKVSCKTSGYTFTEYTMHWVRQAPGQGLEWMG
GINPNNYGAPYNQKFKGRVTMTVDTSISTAYMELSRLRSDDTAVYYCAS
GGYYFDYWGQGTLVTVSS
Hz2D7 Light chain variable region (SEQ ID NO: 22)
DIQMTQSPSSLSASVGDRVTITCKASQDVGTAVAWYQQKPGKAPKLLIY
WAFTRHTGVPSRFSGSGSGTDYTLTISSLQPEDFATYYCQQHYDTPFTF
GQGTKVEIK

TABLE 2
Antibody 2F10 Sequences

| | Exem- | | | | |
|---|---|---|---|---|---|
| | plary | Chothia | AbM | Kabat | Contact |
| Heavy | GYSFT | GYSF | GYSFT | DYIIV | TDYIIV |
| Chain | DYIIV | TDY | DYIIV | (SEQ ID | (SEQ ID |
| Variable | (SEQ ID | (SEQ ID | (SEQ ID | NO: 100) | NO: 101) |
| Region | NO: 27) | NO: 97) | NO: 27) | | |
| CDR1 | | | | | |
| Heavy | KINPYY | NPYYGT | KINPY | KINPY | WIEKIN |
| Chain | GTTTYN | (SEQ ID | YGTTT | YGTTT | PYYGTT |
| Variable | LRFED | NO: 98) | (SEQ ID | YNLRF | T |
| Region | (SEQ ID | | NO: 99) | ED | (SEQ ID |
| CDR2 | NO: 28) | | | (SEQ ID | NO: 102) |
| | | | | NO: 28) | |
| Heavy | WDYVST | WDYVST | WDYVST | WDYVST | ARWDYV |
| Chain | LFAMDY | LFAMDY | LFAMDY | LFAMDY | STLFAM |
| Variable | (SEQ ID | (SEQ ID | (SEQ ID | (SEQ ID | D |
| Region | NO: 29) | NO: 29) | NO: 29) | NO: 29) | (SEQ ID |
| CDR3 | | | | | NO: 103) |
| Light | KASQDV | KASQDV | KASQDV | KASQDV | GTAV |
| Chain | GTAVA | GTAVA | GTAVA | GTAVA | AWY |
| Variable | (SEQ ID | (SEQ ID | (SEQ ID | (SEQ ID | (SEQ ID |
| Region | NO: 16) | NO: 16) | NO: 16) | NO: 16) | NO: 92) |
| CDR1 | | | | | |
| Light | WAST | WASTRHT | WAST | WAST | LLIYW |
| Chain | RHT | (SEQ ID | RHT | RHT | ASTRH |
| Variable | (SEQ ID | NO 30) | (SEQ ID | (SEQ ID | (SEQ ID |
| Region | NO: 30) | | NO: 30) | NO: 30) | NO: 104) |
| CDR2 | | | | | |
| Light | QQYKR | QQYK | QQYK | QQYKR | QQYK |
| Chain | YYT | RYYT | RYYT | YYT | RYY |
| Variable | (SEQ ID | (SEQ ID | (SEQ ID | (SEQ ID | (SEQ ID |
| Region | NO: 31) | NO: 31) | NO: 31) | NO: 31) | NO: 105) |
| CDR3 | | | | | |

2F10 Heavy chain variable region (SEQ ID NO: 32)
EIQLQQTGPELVKPGASLRISCKASGYSFTDYIIVWVKQNQGKNLDW
IEKINPYYGTTTYNLRFEDKATLTVNKSSTTAYMQLNSLTSEDSAVY
YCARWDYVSTLFAMDYWGQGTSITVSS
2F10 Light chain variable region (SEQ ID NO: 33)
DIVMTQSHKVMSTSVGDRVSIICKASQDVGTAVAWYQQKPGQSPKLL
IYWASTRHTGVPDRFTGSGSGTDFILTITNVQSENLANYFCQQYKRY
YTFGGGTKLEIK

TABLE 3
Antibody 3H6 Sequences

| | Exem- | | | | |
|---|---|---|---|---|---|
| | plary | Chothia | AbM | Kabat | Contact |
| Heavy | GYTFT | GYTFTEY | GYTFT | EYTMH | TEYTMH |
| Chain | EYTMH | (SEQ ID | EYTMH | (SEQ ID | (SEQ ID |
| Variable | (SEQ ID | NO: 85) | (SEQ ID | NO: 88) | NO: 89) |
| Region | NO: 13) | | NO: 13) | | |
| CDR1 | | | | | |
| Heavy | GINPNN | NPNNGG | GINPN | GINPNNG | WIGGIN |
| Chain | GGAPYN | (SEQ ID | NGGAP | GAPYNQK | PNNGGAP |
| Variable | QKFKG | NO: 106) | (SEQ ID | FKG | (SEQ ID |
| Region | (SEQ ID | | NO: 107) | (SEQ ID | NO: 108) |
| CDR2 | NO: 34) | | | NO: 34) | |
| Heavy | GGYYFDY | GGYYFDY | GGYY | GGYYFDY | ARGG |
| Chain | (SEQ ID | (SEQ ID | FDY | (SEQ ID | YYFD |
| Variable | NO: 15) | NO: 15) | (SEQ ID | NO: 15) | (SEQ ID |
| Region | | | NO: 15) | | NO: 109) |
| CDR3 | | | | | |
| Light | KASQDV | KASQDV | KASQDV | KASQDV | STAV |
| Chain | STAVA | STAVA | STAVA | STAVA | AWY |
| Variable | (SEQ ID | (SEQ ID | (SEQ ID | (SEQ ID | (SEQ ID |
| Region | NO: 35) | NO: 35) | NO: 35) | NO: 35) | NO: 110) |
| CDR1 | | | | | |
| Light | WASTRHT | WAST | WAST | WASTRHT | LLIYW |
| Chain | (SEQ ID | RHT | RHT | (SEQ ID | ASTRH |
| Variable | NO: 30) | (SEQ ID | (SEQ ID | NO: 30) | (SEQ ID |
| Region | | NO: 30) | NO: 30) | | NO: 111) |
| CDR2 | | | | | |
| Light | QQHYDT | QQHYD | QQHYD | QQHYD | QQHY |
| Chain | PFT | TPFT | TPFT | TPFT | DTPF |
| Variable | (SEQ ID | (SEQ ID | (SEQ ID | (SEQ ID | (SEQ ID |
| Region | NO: 18) | NO: 18) | NO: 18) | NO: 18) | NO: 94) |
| CDR3 | | | | | |

3H6 Heavy chain variable region (SEQ ID NO: 36)
EVQLQQSGPELVKPGASVKISCKTSGYTFTEYTMHWVKQSHGKSL
EWIGGINPNNGGAPYNQKFKGKATLTVDKSSSTAYMELRSLTSED
SAVYYCARGGYYFDYWGQGTTLTVSS
3H6 Light chain v ariable region (SEQ ID NO: 37)
DIVMTQSHKFMSTSVGDRVSITCKASQDVSTAVAWYQQKSGQSPKL
LIYWASTRHTGVPDRFTGSGSGTDYTLTISSVQAEDLALYYCQQHY
DTPFTFGGGTKLEIK

TABLE 4
Antibody 5F7 Sequences

| | Exem- | | | | |
|---|---|---|---|---|---|
| | plary | Chothia | AbM | Kabat | Contact |
| Heavy | GYTFS | GYTFSSY | GYTFSS | SYSMH | SSYSMH |
| Chain | SYSMH | (SEQ ID | YSMH | (SEQ ID | (SEQ ID |
| Variable | (SEQ ID | NO: 112) | (SEQ ID | NO: 115) | NO: 116) |
| Region | NO: 38) | | NO: 38) | | |
| CDR1 | | | | | |
| Heavy | TIYPG | YPGNEN | TIYPG | TIYPGN | WMGTIY |
| Chain | NENTSY | (SEQ ID | NENTS | ENTSYN | PGNENT |
| Variable | NQKFKG | NO: 113) | (SEQ ID | QKFKG | S |
| Region | (SEQ ID | | NO: 114) | (SEQ ID | (SEQ ID |
| CDR2 | NO: 39) | | | NO: 39) | NO: 117) |
| Heavy | GGYYF | GGYYFDY | GGYY | GGYYFDY | ARGG |
| Chain | DY | (SEQ ID | FDY | (SEQ ID | YYFD |
| Variable | (SEQ ID | NO: 15) | (SEQ ID | NO: 15) | (SEQ ID |
| Region | NO: 15) | | NO: 15) | | NO: 109) |
| CDR3 | | | | | |
| Light | KASQDV | KASQDV | KASQDV | KASQDV | STAV |
| Chain | STAVA | STAVA | STAVA | STAVA | AWY |
| Variable | (SEQ ID | (SEQ ID | (SEQ ID | (SEQ ID | (SEQ ID |
| Region | NO: 35) | NO: 35) | NO: 35) | NO: 35) | NO: 110) |
| CDR1 | | | | | |
| Light | WAST | WASTRHT | WAST | WAST | LLIYWA |
| Chain | RHT | (SEQ ID | RHT | RHT | STRH |
| Variable | (SEQ ID | NO: 30) | (SEQ ID | (SEQ ID | (SEQ ID |
| Region | NO: 30) | | NO: 30) | NO: 30) | NO: 111) |
| CDR2 | | | | | |
| Light | QQHF | QQHFD | QQHFD | QQHFD | QQHF |
| Chain | DIPYW | IPYW | IPYW | IPYW | DIPY |
| Variable | (SEQ ID | (SEQ ID | (SEQ ID | (SEQ ID | (SEQ ID |
| Region | NO: 40) | NO: 40) | NO: 40) | NO: 40) | NO: 118) |
| CDR3 | | | | | |

5F7 Heavy chain variable region (SEQ ID NO: 41)
QEQLQQPGTELVKPGASVKISCKASGYTFSSYSMHWVKLTPGQGLEW
MGTIYPGNENTSYNQKFKGKATLTADKSSSTAYMQLSSLTSEDSAVY
YCARGGYYFDYWGQGTTLTVSS
5F7 Light chain variable region (SEQ ID NO: 42)
DIVMTQSHKFLSTSVGDRVSITCKASQDVSTAVAWYQQKPGQSPKLL
IYWASTRHTGVSDRFTGSGSGTDFTLTISSVQAEDLALYYCQQHFDI
PYWFGGGTKLEIK

TABLE 5
Antibody 16D5 Sequences

| | Exem- | | | | |
|---|---|---|---|---|---|
| | plary | Chothia | AbM | Kabat | Contact |
| Heavy | GYTFT | GYTFTTY | GYTFTT | TYTMH | TTYTMH |
| Chain | TYTMH | (SEQ ID | YTMH | (SEQ ID | (SEQ ID |
| Variable | (SEQ ID | NO: 119) | (SEQ ID | NO: 122) | NO: 123) |
| Region | NO: 43) | | NO: 43) | | |
| CDR1 | | | | | |
| Heavy | YINPSRG | NPSRGY | YINPS | YINPSR | WIGYIN |
| Chain | YSDYSQK | (SEQ ID | RGYSD | GYSDYS | PSRGYSD |
| Variable | FQG | NO: 120) | (SEQ ID | QKFQG | (SEQ ID |
| Region | (SEQ ID | | NO: 121) | (SEQ ID | NO: 124) |
| CDR2 | NO: 44) | | | NO: 44) | |
| Heavy | GGYDFDY | GGYDFDY | GGYD | GGYD | ARGGY |
| Chain | (SEQ ID | (SEQ ID | FDY | FDY | DFD |
| Variable | NO: 45) | NO: 45) | (SEQ ID | (SEQ ID | (SEQ ID |
| Region | | | NO: 45) | NO: 45) | NO: 125) |
| CDR3 | | | | | |
| Light | KASQDV | KASQDV | KASQDV | KASQDV | GTAV |
| Chain | GTAVA | GTAVA | GTAVA | GTAVA | AWY |
| Variable | (SEQ ID | (SEQ ID | (SEQ ID | (SEQ ID | (SEQ ID |
| Region | NO: 16) | NO: 16) | NO: 16) | NO: 16) | NO: 92) |
| CDR1 | | | | | |
| Light | WASTRHT | WASTRHT | WAST | WASTRHT | LLLYW |
| Chain | (SEQ ID | (SEQ ID | RHT | (SEQ ID | ASTRH |
| Variable | NO: 30) | NO: 30) | (SEQ ID | NO: 30) | (SEQ ID |
| Region | | | NO: 30) | | NO: 126) |
| CDR2 | | | | | |
| Light | QQHFI | QQHFI | QQHFI | QQHFI | QQHFI |
| Chain | TPYT | TPYT | TPYT | TPYT | TPY |
| Variable | (SEQ ID | (SEQ ID | (SEQ ID | (SEQ ID | (SEQ ID |
| Region | NO: 46) | NO: 46) | NO: 46) | NO: 46) | NO: 127) |
| CDR3 | | | | | |

16D5 Heavy chain variable region (SEQ ID NO: 47)
QVQLQQSAAELARPGASVKMSCKASGYTFTTYTMHWVKQRPGQGLE
WIGYINPSRGYSDYSQKFQGKSTLTTDKSSNTAYIQLTSLTSEDSA
VYFCARGGYDFDYWGQGTTLTVSS
16D5 Light chain variable region (SEQ ID NO: 48)
DIVMTQSHKFMSTSVGDRVSITCKASQDVGTAVAWYQQKPGQSPKL
LLYWASTRHTGVPDRFTGSGSGTDYTLTISSVQAEDLALYHCQQHF
ITPYTFGGGTKLEIK

TABLE 6
Antibody 28F5 Sequences

| | Exem- | | | | |
|---|---|---|---|---|---|
| | plary | Chothia | AbM | Kabat | Contact |
| Heavy | GFSLT | GFSLTGY | GFSLT | GYGVN | TGYGVN |
| Chain | GYGVN | (SEQ ID | GYGVN | (SEQ ID | (SEQ ID |
| Variable | (SEQ ID | NO: 128) | (SEQ ID | NO: 131) | NO: 132) |
| Region | NO: 49) | | NO: 49) | | |
| CDR1 | | | | | |
| Heavy | VIWGDG | WGDGS | VIWGD | VIWGDG | WLGVIW |
| Chain | STDYNS | (SEQ ID | GSTD | STDYNS | GDGSTD |
| Variable | VLKS | NO: 129) | (SEQ ID | VLKS | (SEQ ID |
| Region | (SEQ ID | | NO: 130) | (SEQ ID | NO: 133) |
| CDR2 | NO: 50) | | | NO: 50) | |
| Heavy | EATEYL | EATEYL | EATEY | EATEYL | AREATEY |
| Chain | YWYFDV | YWYFDV | LYWYF | YWYFDV | LYWYFD |
| Variable | (SEQ ID | (SEQ ID | DV | (SEQ ID | (SEQ ID |
| Region | NO: 51) | NO: 51) | (SEQ ID | NO: 51) | NO: 134) |
| CDR3 | | | NO: 51) | | |
| Light | RASESV | RASESV | RASESV | RASESVE | EYYGS |
| Chain | EYYGSS | EYYGSS | EYYGSS | YYGSSLM | SLMQWY |
| Variable | LMQ | LMQ | LMQ | Q | (SEQ ID |
| Region | (SEQ ID | (SEQ ID | (SEQ ID | (SEQ ID | NO: 135) |
| CDR1 | NO: 52) | NO: 52) | NO: 52) | NO: 52) | |
| Light | AASN | AASN | AASN | AASNVES | LLIFAA |
| Chain | VES | VES | VES | (SEQ ID | SNVE |
| Variable | (SEQ ID | (SEQ ID | (SEQ ID | NO: 53) | (SEQ ID |
| Region | NO: 53) | NO: 53) | NO: 53) | | NO: 136) |
| CDR2 | | | | | |
| Light | QQGRRV | QQGRR | QQGRR | QQGRRV | QQGRR |
| Chain | PWT | VPWT | VPWT | PWT | VPW |
| Variable | (SEQ ID | (SEQ ID | (SEQ ID | (SEQ ID | (SEQ ID |
| Region | NO: 54) | NO: 54) | NO: 54) | NO: 54) | NO: 137) |
| CDR3 | | | | | |

28F5 Heavy chain variable region (SEQ ID NO: 55)
QVQLKESGPGLVAPSQSLSITCTVSGFSLTGYGVNWVRQPPGKGLEW
LGVIWGDGSTDYNSVLKSRLSISKDNSKSQVFLKMNSLQTDDTARYY
CAREATEYLYWYFDVWGAGTTVTVSS
28F5 Light chain variable region (SEQ ID NO: 56)
DIVLTQSPASLAVSLGQRATISCRASESVEYYGSSLMQWYQQKPGQP
PKLLIFAASNVESGVPPRFSGSGSGTDFNLN1HPVDEDD1AMYFCQQ
GRRVPWTFGGGTKLEIK

TABLE 7
Antibody 1D3 Sequences

| | Exem- | | | | |
|---|---|---|---|---|---|
| | plary | Chothia | AbM | Kabat | Contact |
| Heavy | GYSFT | GYSFTGY | GYSFT | GYNMN | TGYNMN |
| Chain | GYNMN | (SEQ ID | GYNMN | (SEQ ID | (SEQ ID |
| Variable | (SEQ ID | NO: 138) | (SEQ ID | NO: 141) | NO: 142) |
| Region | NO: 57) | | NO: 57) | | |
| CDR1 | | | | | |
| Heavy | NIDPYS | DPYSGG | NIDPY | NIDPYS | WIGNID |
| Chain | GGSTYN | (SEQ ID | SGGST | GGSTYN | PYSGGS |
| Variable | QKFKG | NO: 139) | (SEQ ID | QKFKG | T |
| Region | (SEQ ID | | NO: 140) | (SEQ ID | (SEQ ID |
| CDR2 | NO: 58) | | | NO: 58) | NO: 143) |
| Heavy | SVYDA | SVYDA | SVYDAP | SVYDA | ARSVYD |
| Chain | PWLAH | PWLAH | WLAH | PWLAH | APWLA |
| Variable | (SEQ ID | (SEQ ID | (SEQ ID | (SEQ ID | (SEQ ID |
| Region | NO: 59) | NO: 59) | NO: 59) | NO: 59) | NO: 144) |
| CDR3 | | | | | |
| Light | RASENI | RASENI | RASENI | RASENI | YIYLAWY |
| Chain | YIYLA | YIYLA | YIYLA | YIYLA | (SEQ ID |
| Variable | (SEQ ID | (SEQ ID | (SEQ ID | (SEQ ID | NO: 145) |
| Region | NO: 60) | NO: 60) | NO: 60) | NO: 60) | |
| CDR1 | | | | | |
| Light | NAKT | NAKTLAE | NAKTLAE | NAKT | LLVYNA |
| Chain | LAE | (SEQ ID | (SEQ ID | LAE | KTLA |
| Variable | (SEQ ID | NO: 61) | NO: 61) | (SEQ ID | (SEQ ID |
| Region | NO: 61) | | | NO: 61) | NO: 146) |
| CDR2 | | | | | |
| Light | QHHYG | QHHYG | QHHY | QHHYG | QHHY |
| Chain | TPPT | TPPT | GTPPT | TPPT | GTPP |
| Variable | (SEQ ID | (SEQ ID | (SEQ ID | (SEQ ID | (SEQ ID |
| Region | NO: 62) | NO: 62) | NO: 62) | NO: 62) | NO: 147) |
| CDR3 | | | | | |

1D3 Heavy chain variable region (SEQ ID NO: 63)
EVQLQQSGPELEKPGASVKISCKASGYSFTGYNMNWVKESNGRSLE
WIGN1DPYSGGSTYNQKFKGKATMTVDKSSSTAYMQLKSLTSEDSA
VYYCARSVYDAPWLAHWGQGTLVTVSA
1D3 Light chain variable region (SEQ ID NO: 64)
DIQMTQSPASLSASVGETVTITCRASENIYIYLAWYQQKQGKSPQL
LVYNAKTLAEGVPSRFSGSGSGTQFSLKINRLQPEDFGNYYCQHHY
GTPPTFGGGTKLEIK

TABLE 8
Antibody 5B10 Sequences

| | Exem- | | | | |
|---|---|---|---|---|---|
| | plary | Chothia | AbM | Kabat | Contact |
| Heavy | GYSFTG | GYSFTGY | GYSFTG | GYNMN | TGYNMN |
| Chain | YNMN | (SEQ ID | YNMN | (SEQ ID | (SEQ ID |
| Variable | (SEQ ID | NO: 138) | (SEQ ID | NO: 141) | NO: 142) |
| Region | NO: 57) | | NO: 57) | | |
| CDR1 | | | | | |
| Heavy | NIDPYS | DPYSGG | NIDPYS | NIDPYSG | WIGNIDP |
| Chain | GGSTYN | (SEQ ID | GGST | GSTYNQK | YSGGST |
| Variable | QKFKG | NO: 139) | (SEQ ID | FKG | (SEQ ID |
| Region | (SEQ ID | | NO: 140) | (SEQ ID | NO: 143) |
| CDR2 | NO: 58) | | | NO: 58) | |
| Heavy | SFYDAP | SFYDA | SFYDAP | SFYDAP | ARSFYD |
| Chain | YLTY | PYLTY | YLTY | YLTY | APYLT |
| Variable | (SEQ ID | (SEQ ID | (SEQ ID | (SEQ ID | (SEQ ID |
| Region | NO: 65) | NO: 65) | NO: 65) | NO: 65) | NO: 148) |
| CDR3 | | | | | |
| Light | RASENI | RASENI | RASENI | RASENI | FIYLA |
| Chain | FIYLA | FIYLA | FIYLA | FIYLA | WY |
| Variable | (SEQ ID | (SEQ ID | (SEQ ID | (SEQ ID | (SEQ ID |
| Region | NO: 66) | NO: 66) | NO: 66) | NO: 66) | NO: 149) |
| CDR1 | | | | | |
| Light | NAKTLAE | NAKTLAE | NAKTLAE | NAKTLAE | LLVFNA |
| Chain | (SEQ ID | (SEQ ID | (SEQ ID | (SEQ ID | KTLA |
| Variable | NO: 61) | NO: 61) | NO: 61) | NO: 61) | (SEQ ID |
| Region | | | | | NO: 150) |
| CDR2 | | | | | |
| Light | QHHY | QHHYG | QHHY | QHHYG | QHHY |
| Chain | GTPPT | TPPT | GTPPT | TPPT | GTPP |
| Variable | (SEQ ID | (SEQ ID | (SEQ ID | (SEQ ID | (SEQ ID |
| Region | NO: 62) | NO: 62) | NO: 62) | NO: 62) | NO: 147) |
| CDR3 | | | | | |

5B10 Heavy chain variable region (SEQ ID NO: 67)
EVQLQQSGPELEKPGASVKISCKASGYSFTGYNMNWVKQSNGKSLEW
IGNIDPYSGGSTYNQKFKGKATLTVDKSSSTASLQLKSLTSEDSSVY
YCARSFYDAPYLTYWGQGTLVTVSA
5B10 Light chain variable region (SEQ ID NO: 68)
DIQMTQSPTSLSASVGETVTITCRASENIFIYLAWYQQKQGKSPQLL
VFNAKTLAEGVPSRFSGSGSGTQFSLKINSLRPEDFGTYYCQHHYGT
PPTFGGGTKLEV

TABLE 9
Antibody 5C9 Sequences

| | Exem- | | | | |
|---|---|---|---|---|---|
| | plary | Chothia | AbM | Kabat | Contact |
| Heavy | GYTFT | GYTFTSF | GYTFT | SFWIH | TSFWIH |
| Chain | SFWIH | (SEQ ID | SFWIH | (SEQ ID | (SEQ ID |
| Variable | (SEQ ID | NO: 151) | (SEQ ID | NO: 154) | NO: 155) |
| Region | NO: 69) | | NO: 69) | | |
| CDR1 | | | | | |
| Heavy | YIIPNT | IPNTDY | YIIPN | YIIPN | WIGYII |
| Chain | DYTEYN | (SEQ ID | TDYTE | TDYTE | PNTDYTE |
| Variable | QKFKD | NO: 152) | (SEQ ID | YNQKF | (SEQ ID |
| Region | (SEQ ID | | NO: 153) | KD | NO: 156) |
| CDR2 | NO: 70) | | | (SEQ ID | |
| | | | | NO: 70) | |
| Heavy | GLRGA | GLRGAY | GLRGAY | GLRGA | ARGLRG |
| Chain | YYFDY | YFDY | YFDY | YYFDY | AYYFD |
| Variable | (SEQ ID | (SEQ ID | (SEQ ID | (SEQ ID | (SEQ ID |
| Region | NO: 71) | NO: 71) | NO: 71) | NO: 71) | NO: 157) |
| CDR3 | | | | | |
| Light | RSSQSV | RSSQSV | RSSQSVS | RSSQSV | STSTNG |
| Chain | STSTNG | STSTNG | TSTNGYM | STSTNG | YMHWY |
| Variable | YMH | YMH | H | YMH | (SEQ ID |
| Region | (SEQ ID | (SEQ ID | (SEQ ID | (SEQ ID | NO: 158) |
| CDR1 | NO:72) | NO: 72) | NO: 72) | NO: 72) | |
| Light | YASN | YASNLES | YASNLES | YASNLES | LLIMY |
| Chain | LES | (SEQ ID | (SEQ ID | (SEQ ID | ASNLE |
| Variable | (SEQ ID | NO: 73) | NO: 73) | NO: 73) | (SEQ ID |
| Region | NO:73) | | | | NO: 159) |
| CDR2 | | | | | |
| Light | QHSWV | QHSWVL | QHSWV | QHSWV | QHSWV |
| Chain | LPYT | PYT | LPYT | LPYT | LPY |
| Variable | (SEQ ID | (SEQ ID | (SEQ ID | (SEQ ID | (SEQ ID |
| Region | NO: 74) | NO: 74) | NO: 74) | NO: 74) | NO: 160) |
| CDR3 | | | | | |

5C9 Heavy chain variable region (SEQ ID NO: 75)
QVQLQQSGAELAKPGASVKMSCKASGYTFTSFWIHWIKQRPGQGLE
WIGYIIPNTDYTEYNQKFKDKATFTADKSSSTAYMQLSSLTSEDSA
VFYCARGLRGAYYFDYWGQGTTLTVSS
5C9 Light chain variable region (SEQ ID NO: 76)
DIVLTQSPASLAVSLGLRATISCRSSQSVSTSTNGYMHWYQQKPGQ
PPKLLIMYASNLESGVPARFSGSGSGTDFTLN1HPVDEEDTATYYC
QHSWVLPYTFGGGTKLEIK

In some embodiments, a B7-H7-binding agent comprises a heavy chain variable region CDR1, CDR2, and CDR3 and/or a light chain variable region CDR1, CDR2, and CDR3 from an antibody described herein. In some embodiments, a B7-H7-binding agent comprises a heavy chain variable region CDR1, CDR2, and CDR3 and a light chain variable region CDR1, CDR2, and CDR3 from an antibody described herein. In some embodiments, a B7-H7-binding agent comprises a humanized version or humanized variant of an antibody described herein.

In some embodiments, a B7-H7-binding agent comprises a heavy chain variable region CDR1, CDR2, and CDR3 and/or a light chain variable region CDR1, CDR2, and CDR3 from antibody 2D7, a humanized version thereof, or variants thereof. In some embodiments, a B7-H7-binding agent comprises a heavy chain variable region CDR1, a heavy chain variable region CDR2, and a heavy chain variable region CDR3 from antibody 2D7. In other embodiments, a B7-H7-binding agent comprises a light chain variable region CDR1, a light chain variable region CDR2, and a light chain variable region CDR3 from antibody 2D7. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 2D7. In some embodiments, a B7-H7-binding agent is a humanized version of antibody 2D7 (e.g., Hz2D7). In some embodiments, a B7-H7-binding agent is a variant of antibody 2D7.

In some embodiments, a B7-H7-binding agent comprises a heavy chain variable region CDR1, CDR2, and CDR3 and/or a light chain variable region CDR1, CDR2, and CDR3 from antibody 2F10, a humanized version thereof, or variants thereof. In some embodiments, a B7-H7-binding agent comprises a heavy chain variable region CDR1, a heavy chain variable region CDR2, and a heavy chain variable region CDR3 from antibody 2F10. In other embodiments, a B7-H7-binding agent comprises a light chain variable region CDR1, a light chain variable region CDR2, and a light chain variable region CDR3 from antibody 2F10. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 2F10. In some embodiments, a B7-H7-binding agent is a humanized version of antibody 2F10. In some embodiments, a B7-H7-binding agent is a variant of antibody 2F10.

In some embodiments, a B7-H7-binding agent comprises a heavy chain variable region CDR1, CDR2, and CDR3 and/or a light chain variable region CDR1, CDR2, and CDR3 from antibody 3H6, a humanized version thereof, or variants thereof. In some embodiments, a B7-H7-binding agent comprises a heavy chain variable region CDR1, a heavy chain variable region CDR2, and a heavy chain variable region CDR3 from antibody 3H6. In other embodiments, a B7-H7-binding agent comprises a light chain variable region CDR1, a light chain variable region CDR2, and a light chain variable region CDR3 from antibody 3H6. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 3H6. In some embodiments, a B7-H7-binding agent is a humanized version of antibody 3H6. In some embodiments, a B7-H7-binding agent is a variant of antibody 3H6.

In some embodiments, a B7-H7-binding agent comprises a heavy chain variable region CDR1, CDR2, and CDR3 and/or a light chain variable region CDR1, CDR2, and CDR3 from antibody 5F7, a humanized version thereof, or variants thereof. In some embodiments, a B7-H7-binding agent comprises a heavy chain variable region CDR1, a heavy chain variable region CDR2, and a heavy chain variable region CDR3 from antibody 5F7. In other embodiments, a B7-H7-binding agent comprises a light chain variable region CDR1, a light chain variable region CDR2, and a light chain variable region CDR3 from antibody 5F7. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 5F7. In some embodiments, a B7-H7-binding agent is a humanized version of antibody 5H7. In some embodiments, a B7-H7-binding agent is a variant of antibody 5H7.

In some embodiments, a B7-H7-binding agent comprises a heavy chain variable region CDR1, CDR2, and CDR3 and/or a light chain variable region CDR1, CDR2, and CDR3 from antibody 16D5, a humanized version thereof, or variants thereof. In some embodiments, a B7-H7-binding agent comprises a heavy chain variable region CDR1, a heavy chain variable region CDR2, and a heavy chain variable region CDR3 from antibody 16D5. In other embodiments, a B7-H7-binding agent comprises a light chain variable region CDR1, a light chain variable region CDR2, and a light chain variable region CDR3 from antibody 16D5. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 16D5. In some embodiments, a B7-H7-binding agent is a humanized version of antibody 16D5. In some embodiments, a B7-H7-binding agent is a variant of antibody 16D5.

In some embodiments, a B7-H7-binding agent comprises a heavy chain variable region CDR1, CDR2, and CDR3 and/or a light chain variable region CDR1, CDR2, and CDR3 from antibody 28F5, a humanized version thereof, or variants thereof. In some embodiments, a B7-H7-binding agent comprises a heavy chain variable region CDR1, a heavy chain variable region CDR2, and a heavy chain variable region CDR3 from antibody 28F5. In other embodiments, a B7-H7-binding agent comprises a light chain variable region CDR1, a light chain variable region CDR2, and a light chain variable region CDR3 from antibody 28F5. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 28F5. In some embodiments, a B7-H7-binding agent is a humanized version of antibody 28F5. In some embodiments, a B7-H7-binding agent is a variant of antibody 28F5.

In some embodiments, a B7-H7-binding agent comprises a heavy chain variable region CDR1, CDR2, and CDR3 and/or a light chain variable region CDR1, CDR2, and CDR3 from antibody 1D3, a humanized version thereof, or variants thereof. In some embodiments, a B7-H7-binding agent comprises a heavy chain variable region CDR1, a heavy chain variable region CDR2, and a heavy chain variable region CDR3 from antibody 1D3. In other embodiments, a B7-H7-binding agent comprises a light chain variable region CDR1, a light chain variable region CDR2, and a light chain variable region CDR3 from antibody 1D3. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 1D3. In some embodiments, a B7-H7-binding agent is a humanized version of antibody 1D3. In some embodiments, a B7-H7-binding agent is a variant of antibody 1D3.

In some embodiments, a B7-H7-binding agent comprises a heavy chain variable region CDR1, CDR2, and CDR3 and/or a light chain variable region CDR1, CDR2, and CDR3 from antibody 5B10, a humanized version thereof, or variants thereof. In some embodiments, a B7-H7-binding agent comprises a heavy chain variable region CDR1, a heavy chain variable region CDR2, and a heavy chain variable region CDR3 from antibody 5B10. In other embodiments, a B7-H7-binding agent comprises a light chain variable region CDR1, a light chain variable region CDR2, and a light chain variable region CDR3 from antibody 5B10. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 5B10. In some embodiments, a B7-H7-binding agent is a humanized version of antibody 5B10. In some embodiments, a B7-H7-binding agent is a variant of antibody 5B10.

In some embodiments, a B7-H7-binding agent comprises a heavy chain variable region CDR1, CDR2, and CDR3 and/or a light chain variable region CDR1, CDR2, and CDR3 from antibody 5C9, a humanized version thereof, or variants thereof. In some embodiments, a B7-H7-binding agent comprises a heavy chain variable region CDR1, a heavy chain variable region CDR2, and a heavy chain variable region CDR3 from antibody 5C9. In other embodiments, a B7-H7-binding agent comprises a light chain variable region CDR1, a light chain variable region CDR2, and a light chain variable region CDR3 from antibody 5C9. In certain embodiments, a B7-H7-binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 5C9. In some embodiments, a B7-H7-binding agent is a humanized version of antibody 5C9. In some embodiments, a B7-H7-binding agent is a variant of antibody 5C9.

In some embodiments, a B7-H7-binding agent is an antibody. In some embodiments, a variant of an anti-B7-H7 antibody described herein comprises one to thirty conservative amino acid substitutions. In some embodiments, a variant of the anti-B7-H7 antibody comprises one to twenty-five conservative amino acid substitutions. In some embodiments, a variant of the anti-B7-H7 antibody comprises one to twenty conservative amino acid substitutions. In some embodiments, a variant of the anti-B7-H7 antibody comprises one to fifteen conservative amino acid substitutions. In some embodiments, a variant of the anti-B7-H7 antibody comprises one to ten conservative amino acid substitution(s). In some embodiments, a variant of the anti-B7-H7 antibody comprises one to five conservative amino acid substitution(s). In some embodiments, a variant of the anti-B7-H7 antibody comprises one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

In some embodiments, a B7-H7-binding agent (e.g., an antibody) comprises: (a) a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15); and/or (b) a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, a B7-H7-binding agent comprises a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15). In some embodiments, a B7-H7-binding agent comprises a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, a B7-H7-binding agent comprises (a) a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15), and (b) a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, a B7-H7-binding agent comprises (a) a heavy chain variable region comprising a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15), and (b) a light chain variable region comprising a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18).

In some embodiments, a B7-H7-binding agent (e.g., an antibody) comprises: (a) a heavy chain variable region comprising: a heavy chain variable region CDR1 comprising GYTFTEY (SEQ ID NO:85), a heavy chain variable region CDR2 comprising NPNNYG (SEQ ID NO:86), and a heavy chain variable region CDR3 comprising GGYYFDY (SEQ ID NO: 15); and/or (b) a light chain variable region comprising: a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:85, SEQ ID NO:86, and SEQ ID NO: 15, respectively; and (b) a light chain variable region comprising: a light chain variable region CDR1, CDR2, and CDR3 comprising the sequences of SEQ ID NO: 16, SEQ ID NO: 17, and SEQ ID NO: 18, respectively. In some embodiments, a B7-H7-binding agent (e.g., an antibody) comprises: (a) a heavy chain variable region comprising: a heavy chain variable region CDR1 comprising GYTFTEYTMH (SEQ ID NO: 13), a heavy chain variable region CDR2 comprising GINPNNYGAP (SEQ ID NO:87), and a heavy chain variable region CDR3 comprising GGYYFDY (SEQ ID NO: 15); and/or (b) a light chain variable region comprising: a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 13, SEQ ID NO:87, and SEQ ID NO: 15, respectively; and (b) a light chain variable region comprising: a light chain variable region CDR1, CDR2, and CDR3 comprising the sequences of SEQ ID NO: 16, SEQ ID NO: 17, and SEQ ID NO: 18, respectively. In some embodiments, a B7-H7-binding agent (e.g., an antibody) comprises: (a) a heavy chain variable region comprising: a heavy chain variable region CDR1 comprising EYTMH (SEQ ID NO:88), a heavy chain variable region CDR2 comprising GINPNNYGAPYNQKFKG (SEQ ID NO: 14), and a heavy chain variable region CDR3 comprising GGYYFDY (SEQ ID NO: 15); and/or (b) a light chain variable region comprising: a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:88, SEQ ID NO: 14, and SEQ ID NO: 15, respectively; and (b) a light chain variable region comprising: a light chain variable region CDR1, CDR2, and CDR3 comprising the sequences of SEQ ID NO: 16, SEQ ID NO: 17, and SEQ ID NO: 18, respectively. In some embodiments, a B7-H7-binding agent (e.g., an antibody) comprises: (a) a heavy chain variable region comprising: a heavy chain variable region CDR1 comprising TEYTMH (SEQ ID NO:89), a heavy chain variable region CDR2 comprising WIGGINPNNYGAP (SEQ ID NO:90), and a heavy chain variable region CDR3 comprising ASGGYYFD (SEQ ID NO:91); and/or (b) a light chain variable region comprising: a light chain variable region CDR1 comprising GTAVAWY (SEQ ID NO:92), a light chain variable region CDR2 comprising LLISWAFTRH (SEQ ID NO:93), and a light chain variable region CDR3 comprising QQHYDTPF (SEQ ID NO:94). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:89, SEQ ID NO:90, and SEQ ID NO:91, respectively; and (b) a light chain variable region comprising: a light chain variable region CDR1, CDR2, and CDR3 comprising the sequences of SEQ ID NO:92, SEQ ID NO:93, and SEQ ID NO:94, respectively. In some embodiments, a B7-H7-binding agent (e.g., an antibody) comprises: (a) a heavy chain variable region comprising: a heavy chain variable region CDR1 comprising TEYTMH (SEQ ID NO:89), a heavy chain variable region CDR2 comprising WMGGINPNNYGAP (SEQ ID NO:95), and a heavy chain variable region CDR3 comprising ASGGYYFD (SEQ ID NO:91); and/or (b) a light chain variable region comprising: a light chain variable region CDR1 comprising GTAVAWY (SEQ ID NO:92), a light chain variable region CDR2 comprising LLIYWAFTRH (SEQ ID NO:96), and a light chain variable region CDR3 comprising QQHYDTPF (SEQ ID NO:94). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:89, SEQ ID NO:95, and SEQ ID NO:91, respectively; and (b) a light chain variable region comprising: a light chain variable region CDR1, CDR2, and CDR3 comprising the sequences of SEQ ID NO:92, SEQ ID NO:96, and SEQ ID NO:94, respectively.

In some embodiments, a B7-H7-binding agent (e.g., an antibody) comprises: (a) a heavy chain variable region CDR1 comprising GYTFTEYTMH (SEQ ID NO: 13), or a variant thereof comprising 1, 2, 3, or 4 amino acid substitutions; a heavy chain variable region CDR2 comprising GINPNNYGAPYNQKFKG (SEQ ID NO: 14), or a variant thereof comprising 1, 2, 3, or 4 amino acid substitutions; a heavy chain variable region CDR3 comprising GGYYFDY (SEQ ID NO: 15), or a variant thereof comprising 1, 2, 3, or 4 amino acid substitutions; a light chain variable region CDR1 comprising KASQDVGTAVA (SEQ ID NO: 16), or a variant thereof comprising 1, 2, 3, or 4 amino acid substitutions; a light chain variable region CDR2 comprising WAFTRHT (SEQ ID NO: 17), or a variant thereof comprising 1, 2, 3, or 4 amino acid substitutions; and a light chain variable region CDR3 comprising QQHYDTPFT (SEQ ID NO: 18), or a variant thereof comprising 1, 2, 3, or 4 amino acid substitutions. In some embodiments, a CDR comprises one amino acid substitution. In some embodiments, a CDR comprises two amino acid substitutions. In some embodiments, a CDR comprises three amino acid substitutions. In some embodiments, a CDR comprises four amino acid substitutions. In some embodiments, the one or more amino acid substitutions are conservative substitutions. In some embodiments, the CDR is a heavy chain variable region CDR1. In some embodiments, the CDR is a heavy chain variable region CDR2. In some embodiments, the CDR is a heavy chain variable region CDR3. In some embodiments, the CDR is a light chain variable region CDR1. In some embodiments, the CDR is a light chain variable region CDR2. In some embodiments, the CDR is a light chain variable region CDR3. In some embodiments, the one or more substitutions are made as part of a humanization process. In some embodiments, the one or more substitutions are made as part of a germline humanization process. In some embodiments, the one or more substitutions are made as part of an affinity maturation process. In some embodiments, the one or more substitutions are made as part of an optimization process.

In some embodiments, an anti-B7-H7 binding agent (e.g., antibody) comprises a heavy chain variable region comprising an amino acid sequence that has the three heavy chain variable region CDRs of antibody 2D7 and which has at least 75%, 80%, 85%, 90%, 95%, 96%, 97%, 98%, 99%, or 100% identity to the sequence of SEQ ID NO: 19 and a light chain variable region comprising an amino acid sequence that has the three light chain variable region CDRs of antibody 2D7 and which has at least 75%, 80%, 85%, 90%, 95%, 96%, 97%, 98%, 99%, or 100% identity to the sequence of SEQ ID NO:20. In some embodiments, an anti-B7-H7 binding agent (e.g., antibody) comprises a heavy chain variable region comprising an amino acid sequence that has the three heavy chain variable region CDRs of antibody 2D7 and which has at least 75%, 80%, 85%, 90%, 95%, 96%, 97%, 98%, 99%, or 100% identity to the sequence of SEQ ID NO:21 and a light chain variable region comprising an amino acid sequence that has the three light chain variable region CDRs of antibody 2D7 and which has at least 75%, 80%, 85%, 90%, 95%, 96%, 97%, 98%, 99%, or 100% identity to the sequence of SEQ ID NO:22.

In some embodiments, a B7-H7-binding agent (e.g., an antibody) comprises a heavy chain variable region having at least about 80% sequence identity to SEQ ID NO: 19 or SEQ ID NO:21, and/or a light chain variable region having at least 80% sequence identity to SEQ ID NO:20 or SEQ ID NO:22. In some embodiments, a B7-H7-binding agent (e.g., an antibody) comprises a heavy chain variable region having at least about 85%, at least about 90%, at least about 95%, at least about 97%, or at least about 99% sequence identity to SEQ ID NO: 19. In some embodiments, a B7-H7-binding agent (e.g., an antibody) comprises a light chain variable region having at least about 85%, at least about 90%, at least about 95%, at least about 97%, or at least about 99% sequence identity to SEQ ID NO:20. In some embodiments, a B7-H7-binding agent (e.g., an antibody) comprises a heavy chain variable region having at least about 85%, at least about 90%, at least about 95%, at least about 97%, or at least about 99% sequence identity to SEQ ID NO:21. In some embodiments, a B7-H7-binding agent (e.g., an antibody) comprises a light chain variable region having at least about 85%, at least about 90%, at least about 95%, at least about 97%, or at least about 99% sequence identity to SEQ ID NO:22.

In some embodiments, a B7-H7-binding agent (e.g., an antibody) comprises a heavy chain variable region having at least about 80% sequence identity to SEQ ID NO: 19 and/or a light chain variable region having at least about 80% sequence identity to SEQ ID NO:20. In some embodiments, a B7-H7-binding agent (e.g., an antibody) comprises a heavy chain variable region having at least about 80% sequence identity to SEQ ID NO: 19 and a light chain variable region having at least about 80% sequence identity to SEQ ID NO:20. In some embodiments, a B7-H7-binding agent (e.g., an antibody) comprises a heavy chain variable region having at least about 90% sequence identity to SEQ ID NO: 19 and/or a light chain variable region having at least about 90% sequence identity to SEQ ID NO:20. In some embodiments, a B7-H7-binding agent (e.g., an antibody) comprises a heavy chain variable region having at least about 90% sequence identity to SEQ ID NO: 19 and a light chain variable region having at least about 90% sequence identity to SEQ ID NO:20. In some embodiments, a B7-H7-binding agent (e.g., an antibody) comprises a heavy chain variable region having at least about 95% sequence identity to SEQ ID NO: 19 and/or a light chain variable region having at least about 95% sequence identity to SEQ ID NO:20. In some embodiments, a B7-H7-binding agent (e.g., an antibody) comprises a heavy chain variable region having at least about 95% sequence identity to SEQ ID NO: 19 and a light chain variable region having at least about 95% sequence identity to SEQ ID NO:20.

In some embodiments, a B7-H7-binding agent (e.g., an antibody) comprises a heavy chain variable region comprising SEQ ID NO: 19. In some embodiments, a B7-H7-binding agent comprises a light chain variable region comprising SEQ ID NO:20. In some embodiments, a B7-H7-binding agent (e.g., an antibody) comprises a heavy chain variable region comprising SEQ ID NO: 19 and a light chain variable region comprising SEQ ID NO:20.

In some embodiments, a B7-H7-binding agent (e.g., an antibody) comprises a heavy chain variable region having at least about 80% sequence identity to SEQ ID NO:21 and/or a light chain variable region having at least about 80% sequence identity to SEQ ID NO:22. In some embodiments, a B7-H7-binding agent (e.g., an antibody) comprises a heavy chain variable region having at least about 80% sequence identity to SEQ ID NO:21 and a light chain variable region having at least about 80% sequence identity to SEQ ID NO:22. In some embodiments, a B7-H7-binding agent (e.g., an antibody) comprises a heavy chain variable region having at least about 90% sequence identity to SEQ ID NO:21 and/or a light chain variable region having at least about 90% sequence identity to SEQ ID NO:22. In some embodiments, a B7-H7-binding agent (e.g., an antibody) comprises a heavy chain variable region having at least about 90% sequence identity to SEQ ID NO:21 and a light chain variable region having at least about 90% sequence identity to SEQ ID NO:22. In some embodiments, a B7-H7-binding agent (e.g., an antibody) comprises a heavy chain variable region having at least about 95% sequence identity to SEQ ID NO:21 and/or a light chain variable region having at least about 95% sequence identity to SEQ ID NO:22. In some embodiments, a B7-H7-binding agent (e.g., an antibody) comprises a heavy chain variable region having at least about 95% sequence identity to SEQ ID NO:21 and a light chain variable region having at least about 95% sequence identity to SEQ ID NO:22.

In some embodiments, a B7-H7-binding agent (e.g., an antibody) comprises a heavy chain variable region comprising SEQ ID NO:21. In some embodiments, a B7-H7-binding agent comprises a light chain variable region comprising SEQ ID NO:22. In some embodiments, a B7-H7-binding agent (e.g., an antibody) comprises a heavy chain variable region comprising SEQ ID NO:21 and a light chain variable region comprising SEQ ID NO:22.

In some embodiments, a B7-H7-binding agent (e.g., an antibody) comprises a CDR1, CDR2, and CDR3 from a heavy chain variable region having the amino acid sequence of SEQ ID NO: 19 and a CDR1, CDR2, and CDR3 from a light chain variable region having the amino acid sequence of SEQ ID NO:20. In some embodiments, a B7-H7-binding agent (e.g., an antibody) comprises a CDR1, CDR2, and CDR3 from a heavy chain variable region having the amino acid sequence of SEQ ID NO:21 and a CDR1, CDR2, and CDR3 from a light chain variable region having the amino acid sequence of SEQ ID NO:22. In some embodiments, a B7-H7-binding agent (e.g., an antibody) comprises: (a) a heavy chain variable region comprising a CDR1, CDR2, and CDR3 from a heavy chain variable region having the amino acid sequence of SEQ ID NO: 19, and (b) a light chain variable region comprising a CDR1, CDR2, and CDR3 from a light chain variable region having the amino acid sequence of SEQ ID NO:20. In some embodiments, a B7-H7-binding agent (e.g., an antibody) comprises: (a) a heavy chain variable region comprising a CDR1, CDR2, and CDR3 from a heavy chain variable region having the amino acid sequence of SEQ ID NO:2I, and (b) a light chain variable region comprising a CDR1, CDR2, and CDR3 from a light chain variable region having the amino acid sequence of SEQ ID NO:22.

In some embodiments, a B7-H7-binding agent (e.g., an antibody) comprises (a) a heavy chain variable region CDR1 comprising GYSFTDYIIV (SEQ ID NO:27), a heavy chain variable region CDR2 comprising KINPYYGTTTYNLRFED (SEQ ID NO:28), and a heavy chain variable region CDR3 comprising WDYVSTLFAMDY (SEQ ID NO:29), and/or (b) a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, a B7-H7-binding agent comprises a heavy chain variable region CDR1 comprising GYSFTDYIIV (SEQ ID NO:27), a heavy chain variable region CDR2 comprising KINPYYGTTTYNLRFED (SEQ ID NO:28), and a heavy chain variable region CDR3 comprising WDYVSTLFAMDY (SEQ ID NO:29). In some embodiments, a B7-H7-binding agent comprises a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, a B7-H7-binding agent comprises (a) a heavy chain variable region CDR1 comprising GYSFTDYIIV (SEQ ID NO:27), a heavy chain variable region CDR2 comprising KINPYYGTTTYNLRFED (SEQ ID NO:28), and a heavy chain variable region CDR3 comprising WDYVSTLFAMDY (SEQ ID NO:29), and (b) a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, a B7-H7-binding agent comprises (a) a heavy chain variable region comprising a heavy chain variable region CDR1 comprising GYSFTDYIIV (SEQ ID NO:27), a heavy chain variable region CDR2 comprising KINPYYGTTTYNLRFED (SEQ ID NO:28), and a heavy chain variable region CDR3 comprising WDYVSTLFAMDY (SEQ ID NO:29), and (b) a light chain variable region comprising a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31).

In some embodiments, a B7-H7-binding agent (e.g., an antibody) comprises (a) a heavy chain variable region comprising: a heavy chain variable region CDR1 comprising GYSFTDY (SEQ ID NO:97), a heavy chain variable region CDR2 comprising NPYYGT (SEQ ID NO:98), and a heavy chain variable region CDR3 comprising WDYVSTLFAMDY (SEQ ID NO:29), and/or (b) a light chain variable region comprising: a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:97, SEQ ID NO:98, and SEQ ID NO:29, respectively; and (b) a light chain variable region comprising: a light chain variable region CDR1, CDR2, and CDR3 comprising the sequences of SEQ ID NO: 16, SEQ ID NO:30, and SEQ ID NO:31, respectively. In some embodiments, a B7-H7-binding agent (e.g., an antibody) comprises (a) a heavy chain variable region comprising: a heavy chain variable region CDR1 comprising GYSFTDYIIV (SEQ ID NO:27), a heavy chain variable region CDR2 comprising KINPYYGTTT (SEQ ID NO:99), and a heavy chain variable region CDR3 comprising WDYVSTLFAMDY (SEQ ID NO:29), and/or (b) a light chain variable region comprising: a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:27, SEQ ID NO:99, and SEQ ID NO:29, respectively; and (b) a light chain variable region comprising: a light chain variable region CDR1, CDR2, and CDR3 comprising the sequences of SEQ ID NO: 16, SEQ ID NO:30, and SEQ ID NO:31, respectively. In some embodiments, a B7-H7-binding agent (e.g., an antibody) comprises (a) a heavy chain variable region comprising: a heavy chain variable region CDR1 comprising DYIIV (SEQ ID NO: 100), a heavy chain variable region CDR2 comprising KINPYYGTTTYNLRFED (SEQ ID NO:28), and a heavy chain variable region CDR3 comprising WDYVSTLFAMDY (SEQ ID NO:29), and/or (b) a light chain variable region comprising: a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 100, SEQ ID NO:28, and SEQ ID NO:29, respectively; and (b) a light chain variable region comprising: a light chain variable region CDR1, CDR2, and CDR3 comprising the sequences of SEQ ID NO: 16, SEQ ID NO:30, and SEQ ID NO:31, respectively. In some embodiments, a B7-H7-binding agent (e.g., an antibody) comprises (a) a heavy chain variable region comprising: a heavy chain variable region CDR1 comprising TDYIIV (SEQ ID NO: 101), a heavy chain variable region CDR2 comprising WIEKINPYYGTTT (SEQ ID NO: 102), and a heavy chain variable region CDR3 comprising ARWDYVSTLFAMD (SEQ ID NO: 103), and/or (b) a light chain variable region comprising: a light chain variable region CDR1 comprising GTAVAWY (SEQ ID NO:92), a light chain variable region CDR2 comprising LLIYWASTRH (SEQ ID NO: 104), and a light chain variable region CDR3 comprising QQYKRYY (SEQ ID NO: 105). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 101, SEQ ID NO: 102, and SEQ ID NO: 103, respectively; and (b) a light chain variable region comprising: a light chain variable region CDR1, CDR2, and CDR3 comprising the sequences of SEQ ID NO:92, SEQ ID NO: 104, and SEQ ID NO: 105, respectively.

In some embodiments, an anti-B7-H7 binding agent (e.g., antibody) comprises a heavy chain variable region comprising an amino acid sequence that has the three heavy chain variable region CDRs of antibody 2F10 and which has at least 75%, 80%, 85%, 90%, 95%, 96%, 97%, 98%, 99%, or 100% identity to the sequence of SEQ ID NO:32 and a light chain variable region comprising an amino acid sequence that has the three light chain variable region CDRs of antibody 2F10 and which has at least 75%, 80%, 85%, 90%, 95%, 96%, 97%, 98%, 99%, or 100% identity to the sequence of SEQ ID NO: 33.

In some embodiments, a B7-H7-binding agent (e.g., an antibody) comprises a heavy chain variable region having at least about 80%, about 85%, about 90%, or about 95% sequence identity to SEQ ID NO:32 and/or a light chain variable region having at least about 80%, about 85%, about 90%, or about 95% sequence identity to SEQ ID NO:33. In some embodiments, a B7-H7-binding agent (e.g, an antibody) comprises a heavy chain variable region having at least about 80%, about 85%, about 90%, or about 95% sequence identity to SEQ ID NO:32. In some embodiments, a B7H7 binding agent (e.g., an antibody) comprises a light chain variable region having at least about 80%, about 85%, about 90%, or about 95% sequence identity to SEQ ID NO:33. In some embodiments, a B7-H7-binding agent (e.g., an antibody) comprises a heavy chain variable region having at least about 80%, about 85%, about 90%, or about 95% sequence identity to SEQ ID NO:32 and a light chain variable region having at least about 80%, about 85%, about 90%, or about 95% sequence identity to SEQ ID NO:33. In some embodiments, a B7-H7-binding agent comprises a heavy chain variable region comprising SEQ ID NO:32 and/or a light chain variable region comprising SEQ ID NO:33. In some embodiments, a B7-H7-binding agent comprises a heavy chain variable region comprising SEQ ID NO:32. In some embodiments, a B7-H7-binding agent comprises a light chain variable region comprising SEQ ID NO:33. In some embodiments, a B7-H7-binding agent comprises a heavy chain variable region of SEQ ID NO:32 and a light chain variable region of SEQ ID NO:33.

In some embodiments, a B7-H7-binding agent (e.g., an antibody) comprises (a) a heavy chain variable region CDR1 comprising GYTFTEYTMH (SEQ ID NO: 13), a heavy chain variable region CDR2 comprising GINPNNGGAPYNQKFKG (SEQ ID NO:34), and a heavy chain variable region CDR3 comprising GGYYFDY (SEQ ID NO: 15), and/or (b) a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, a B7-H7-binding agent comprises a heavy chain variable region CDR1 comprising GYTFTEYTMH (SEQ ID NO: 13), a heavy chain variable region CDR2 comprising GINPNNGGAPYNQKFKG (SEQ ID NO:34), and a heavy chain variable region CDR3 comprising GGYYFDY (SEQ ID NO: 15). In some embodiments, a B7-H7-binding agent comprises a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, a B7-H7-binding agent comprises (a) a heavy chain variable region CDR1 comprising GYTFTEYTMH (SEQ ID NO: 13), a heavy chain variable region CDR2 comprising GINPNNGGAPYNQKFKG (SEQ ID NO:34), and a heavy chain variable region CDR3 comprising GGYYFDY (SEQ ID NO: 15), and (b) a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, a B7-H7-binding agent comprises (a) a heavy chain variable region comprising a heavy chain variable region CDR1 comprising GYTFTEYTMH (SEQ ID NO: 13), a heavy chain variable region CDR2 comprising GINPNNGGAPYNQKFKG (SEQ ID NO:34), and a heavy chain variable region CDR3 comprising GGYYFDY (SEQ ID NO: 15), and (b) a light chain variable region comprising a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18).

In some embodiments, a B7-H7-binding agent (e.g., an antibody) comprises (a) a heavy chain variable region comprising: a heavy chain variable region CDR1 comprising GYTFTEY (SEQ ID NO: 85), a heavy chain variable region CDR2 comprising NPNNGG (SEQ ID NO: 106), and a heavy chain variable region CDR3 comprising GGYYFDY (SEQ ID NO: 15), and/or (b) a light chain variable region comprising: a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:85, SEQ ID NO: 106, and SEQ ID NO: 15, respectively; and (b) a light chain variable region comprising: a light chain variable region CDR1, CDR2, and CDR3 comprising the sequences of SEQ ID NO:35, SEQ ID NO:30, and SEQ ID NO: 18, respectively. In some embodiments, a B7-H7-binding agent (e.g., an antibody) comprises (a) a heavy chain variable region comprising: a heavy chain variable region CDR1 comprising GYTFTEYTMH (SEQ ID NO: 13), a heavy chain variable region CDR2 comprising GINPNNGGAP (SEQ ID NO: 107), and a heavy chain variable region CDR3 comprising GGYYFDY (SEQ ID NO: 15), and/or (b) a light chain variable region comprising: a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 13, SEQ ID NO: 107, and SEQ ID NO: 15, respectively; and (b) a light chain variable region comprising: a light chain variable region CDR1, CDR2, and CDR3 comprising the sequences of SEQ ID NO:35, SEQ ID NO:30, and SEQ ID NO: 18, respectively. In some embodiments, a B7-H7-binding agent (e.g., an antibody) comprises (a) a heavy chain variable region comprising: a heavy chain variable region CDR1 comprising EYTMH (SEQ ID NO:88), a heavy chain variable region CDR2 comprising GINPNNGGAPYNQKFKG (SEQ ID NO:34), and a heavy chain variable region CDR3 comprising GGYYFDY (SEQ ID NO: 15), and/or (b) a light chain variable region comprising: a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:88, SEQ ID NO:34, and SEQ ID NO: 15, respectively; and (b) a light chain variable region comprising: a light chain variable region CDR1, CDR2, and CDR3 comprising the sequences of SEQ ID NO:35, SEQ ID NO:30, and SEQ ID NO: 18, respectively. In some embodiments, a B7-H7-binding agent (e.g., an antibody) comprises (a) a heavy chain variable region comprising: a heavy chain variable region CDR1 comprising TEYTMH (SEQ ID NO:89), a heavy chain variable region CDR2 comprising WIGGINPNNGGAP (SEQ ID NO: 108), and a heavy chain variable region CDR3 comprising ARGGYYFD (SEQ ID NO: 109), and/or (b) a light chain variable region comprising: a light chain variable region CDR1 comprising STAVAWY (SEQ ID NO: 110), a light chain variable region CDR2 comprising LLIYWASTRH (SEQ ID NO: 111), and a light chain variable region CDR3 comprising QQHYDTPF (SEQ ID NO:94). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:89, SEQ ID NO: 108, and SEQ ID NO: 109, respectively; and (b) a light chain variable region comprising: a light chain variable region CDR1, CDR2, and CDR3 comprising the sequences of SEQ ID NO: 110, SEQ ID NO: 111, and SEQ ID NO:94, respectively.

In some embodiments, an anti-B7-H7 binding agent (e.g., antibody) comprises a heavy chain variable region comprising an amino acid sequence that has the three heavy chain CDRs of antibody 3H6 and which has at least 75%, 80%, 85%, 90%, 95%, 96%, 97%, 98%, 99%, or 100% identity to the sequence of SEQ ID NO:36 and a light chain variable region comprising an amino acid sequence that has the three light chain variable region CDRs of antibody 3H6 and which has at least 75%, 80%, 85%, 90%, 95%, 96%, 97%, 98%, 99%, or 100% identity to the sequence of SEQ ID NO 37.

In some embodiments, a B7-H7-binding agent (e.g., an antibody) comprises a heavy chain variable region having at least about 80%, about 85%, about 90%, or about 95% sequence identity to SEQ ID NO:36 and/or a light chain variable region having at least about 80%, about 85%, about 90%, or about 95% sequence identity to SEQ ID NO:37. In some embodiments, a B7-H7-binding agent (e.g., an antibody) comprises a heavy chain variable region having at least about 80%, about 85%, about 90%, or about 95% sequence identity to SEQ ID NO:36. In some embodiments, a B7H7 binding agent (e.g., an antibody) comprises a light chain variable region having at least about 80%, about 85%, about 90%, or about 95% sequence identity to SEQ ID NO:37. In some embodiments, a B7-H7-binding agent (e.g., an antibody) comprises a heavy chain variable region having at least about 80%, about 85%, about 90%, or about 95% sequence identity to SEQ ID NO:36 and a light chain variable region having at least about 80%, about 85%, about 90%, or about 95% sequence identity to SEQ ID NO:37. In some embodiments, a B7-H7-binding agent comprises a heavy chain variable region comprising SEQ ID NO:36 and/or a light chain variable region comprising SEQ ID NO:37. In some embodiments, a B7-H7-binding agent comprises a heavy chain variable region comprising SEQ ID NO:36. In some embodiments, a B7-H7-binding agent comprises a light chain variable region comprising SEQ ID NO:37. In some embodiments, a B7-H7-binding agent comprises a heavy chain variable region of SEQ ID NO:36 and a light chain variable region of SEQ ID NO:37.

In some embodiments, a B7-H7-binding agent (e.g., an antibody) comprises (a) a heavy chain variable region CDR1 comprising GYTFSSYSMH (SEQ ID NO:38), a heavy chain variable region CDR2 comprising TIYPGNENTSYNQKFKG (SEQ ID NO:39), and a heavy chain variable region CDR3 comprising GGYYFDY (SEQ ID NO: 15), and/or (b) a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40). In some embodiments, a B7-H7-binding agent comprises a heavy chain variable region CDR1 comprising GYTFSSYSMH (SEQ ID NO:38), a heavy chain variable region CDR2 comprising TIYPGNENTSYNQKFKG (SEQ ID NO:39), and a heavy chain variable region CDR3 comprising GGYYFDY (SEQ ID NO: 15). In some embodiments, a B7-H7-binding agent comprises a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40). In some embodiments, a B7-H7-binding agent comprises (a) a heavy chain variable region CDR1 comprising GYTFSSYSMH (SEQ ID NO:38), a heavy chain variable region CDR2 comprising TIYPGNENTSYNQKFKG (SEQ ID NO:39), and a heavy chain variable region CDR3 comprising GGYYFDY (SEQ ID NO: 15), and (b) a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40). In some embodiments, a B7-H7-binding agent comprises (a) a heavy chain variable region comprising a heavy chain variable region CDR1 comprising GYTFSSYSMH (SEQ ID NO:38), a heavy chain variable region CDR2 comprising TIYPGNENTSYNQKFKG (SEQ ID NO:39), and a heavy chain variable region CDR3 comprising GGYYFDY (SEQ ID NO: 15), and (b) a light chain variable region comprising a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40).

In some embodiments, a B7-H7-binding agent (e.g., an antibody) comprises (a) a heavy chain variable region comprising: a heavy chain variable region CDR1 comprising GYTFSSY (SEQ ID NO: 112), a heavy chain variable region CDR2 comprising YPGNEN (SEQ ID NO: 113), and a heavy chain variable region CDR3 comprising GGYYFDY (SEQ ID NO: 15), and/or (b) a light chain variable region comprising: a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 112, SEQ ID NO: 113, and SEQ ID NO: 15, respectively; and (b) a light chain variable region comprising: a light chain variable region CDR1, CDR2, and CDR3 comprising the sequences of SEQ ID NO:35, SEQ ID NO:30, and SEQ ID NO:40, respectively. In some embodiments, a B7-H7-binding agent (e.g., an antibody) comprises (a) a heavy chain variable region comprising: a heavy chain variable region CDR1 comprising GYTFSSYSMH (SEQ ID NO:38), a heavy chain variable region CDR2 comprising TIYPGNENTS (SEQ ID NO: 114), and a heavy chain variable region CDR3 comprising GGYYFDY (SEQ ID NO: 15), and/or (b) a light chain variable region comprising: a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:38, SEQ ID NO: 114, and SEQ ID NO: 15, respectively; and (b) a light chain variable region comprising: a light chain variable region CDR1, CDR2, and CDR3 comprising the sequences of SEQ ID NO:35, SEQ ID NO:30, and SEQ ID NO:40, respectively. In some embodiments, a B7-H7-binding agent (e.g., an antibody) comprises (a) a heavy chain variable region comprising: a heavy chain variable region CDR1 comprising SYSMH (SEQ ID NO: 115), a heavy chain variable region CDR2 comprising TIYPGNENTSYNQKFKG (SEQ ID NO:39), and a heavy chain variable region CDR3 comprising GGYYFDY (SEQ ID NO: 15), and/or (b) a light chain variable region comprising: a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 115, SEQ ID NO:39, and SEQ ID NO: 15, respectively; and (b) a light chain variable region comprising: a light chain variable region CDR1, CDR2, and CDR3 comprising the sequences of SEQ ID NO:35, SEQ ID NO:30, and SEQ ID NO:40, respectively. In some embodiments, a B7-H7-binding agent (e.g., an antibody) comprises (a) a heavy chain variable region comprising: a heavy chain variable region CDR1 comprising SSYSMH (SEQ ID NO: 116), a heavy chain variable region CDR2 comprising WMGTIYPGNENTS (SEQ ID NO: 117), and a heavy chain variable region CDR3 comprising ARGGYYFD (SEQ ID NO: 109), and/or (b) a light chain variable region comprising: a light chain variable region CDR1 comprising STAVAWY (SEQ ID NO: 110), a light chain variable region CDR2 comprising LLIYWASTRH (SEQ ID NO: 111), and a light chain variable region CDR3 comprising QQHFDIPY (SEQ ID NO: 118). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 116, SEQ ID NO: 117, and SEQ ID NO: 109, respectively; and (b) a light chain variable region comprising: a light chain variable region CDR1, CDR2, and CDR3 comprising the sequences of SEQ ID NO: 110, SEQ ID NO:111, and SEQ ID NO: 118, respectively.

In some embodiments, an anti-B7-H7 binding agent (e.g., antibody) comprises a heavy chain variable region comprising an amino acid sequence that has the three heavy chain variable region CDRs of antibody 5F7 and which has at least 75%, 80%, 85%, 90%, 95%, 96%, 97%, 98%, 99%, or 100% identity to the sequence of SEQ ID NO:41 and a light chain variable region comprising an amino acid sequence that has the three light chain variable region CDRs of antibody 5F7 and which has at least 75%, 80%, 85%, 90%, 95%, 96%, 97%, 98%, 99%, or 100% identity to the sequence of SEQ ID NO:42.

In some embodiments, a B7-H7-binding agent (e.g., an antibody) comprises a heavy chain variable region having at least about 80%, about 85%, about 90%, or about 95% sequence identity to SEQ ID NO:41 and/or a light chain variable region having at least about 80%, about 85%, about 90%, or about 95% sequence identity to SEQ ID NO:42, in some embodiments, a B7-H7-binding agent (e.g., an antibody) comprises a heavy chain variable region having at least about 80%, about 85%, about 90%, or about 95% sequence identity to SEQ ID NO:41. In some embodiments, a B7-H7-binding agent (e.g., an antibody) comprises a light chain variable region having at least about 80%, about 85%, about 90%, or about 95% sequence identity to SEQ ID NO:42. In some embodiments, a B7-H7-binding agent (e.g., an antibody) comprises a heavy chain variable region having at least about 80%, about 85%, about 90%, or about 95% sequence identity to SEQ ID NO:4I and a light chain variable region having at least about 80%, about 85%, about 90%, or about 95% sequence identity to SEQ ID NO:42. In some embodiments, a B7-H7-binding agent comprises a heavy chain variable region comprising SEQ ID NO:41 and/or a light chain variable region comprising SEQ ID NO:42. In some embodiments, a B7-H7-binding agent comprises a heavy chain variable region comprising SEQ ID NO:41. In some embodiments, a B7-H7-binding agent comprises a light chain variable region comprising SEQ ID NO:42. In some embodiments, a B7-H7-binding agent comprises a heavy chain variable region of SEQ ID NO:41 and a light chain variable region of SEQ ID NO:42.

In some embodiments, a B7-H7-binding agent (e.g., an antibody) comprises (a) a heavy chain variable region CDR1 comprising GYTFTTYTMH (SEQ ID NO:43), a heavy chain variable region CDR2 comprising YINPSRGYSDYSQKFQG (SEQ ID NO:44), and a heavy chain variable region CDR3 comprising GGYDFDY (SEQ ID NO:45), and/or (b) a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, a B7-H7-binding agent comprises a heavy chain variable region CDR1 comprising GYTFTTYTMH (SEQ ID NO:43), a heavy chain variable region CDR2 comprising YINPSRGYSDYSQKFQG (SEQ ID NO:44), and a heavy chain variable region CDR3 comprising GGYDFDY (SEQ ID NO:45). In some embodiments, a B7-H7-binding agent comprises a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, a B7-H7-binding agent comprises (a) a heavy chain variable region CDR1 comprising GYTFTTYTMH (SEQ ID NO:43), a heavy chain variable region CDR2 comprising YINPSRGYSDYSQKFQG (SEQ ID NO:44), and a heavy chain variable region CDR3 comprising GGYDFDY (SEQ ID NO:45), and (b) a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, a B7-H7-binding agent comprises (a) a heavy chain variable region comprising a heavy chain variable region CDR1 comprising GYTFTTYTMH (SEQ ID NO:43), a heavy chain variable region CDR2 comprising YINPSRGYSDYSQKFQG (SEQ ID NO:44), and a heavy chain variable region CDR3 comprising GGYDFDY (SEQ ID NO:45), and (b) a light chain variable region comprising a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46).

In some embodiments, a B7-H7-binding agent (e.g., an antibody) comprises (a) a heavy chain variable region comprising: a heavy chain variable region CDR1 comprising GYTFTTY (SEQ ID NO: 119), a heavy chain variable region CDR2 comprising NPSRGY (SEQ ID NO: 120), and a heavy chain variable region CDR3 comprising GGYDFDY (SEQ ID NO:45), and/or (b) a light chain variable region comprising: a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 119, SEQ ID NO: 120, and SEQ ID NO:45, respectively; and (b) a light chain variable region comprising: a light chain variable region CDR1, CDR2, and CDR3 comprising the sequences of SEQ ID NO: 16, SEQ ID NO:30, and SEQ ID NO:46, respectively. In some embodiments, a B7-H7-binding agent (e.g., an antibody) comprises (a) a heavy chain variable region comprising: a heavy chain variable region CDR1 comprising GYTFTTYTMH (SEQ ID NO:43), a heavy chain variable region CDR2 comprising YINPSRGYSD (SEQ ID NO: 121), and a heavy chain variable region CDR3 comprising GGYDFDY (SEQ ID NO:45), and/or (b) a light chain variable region comprising: a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:43, SEQ ID NO: 121, and SEQ ID NO:45, respectively; and (b) a light chain variable region comprising: a light chain variable region CDR1, CDR2, and CDR3 comprising the sequences of SEQ ID NO: 16, SEQ ID NO:30, and SEQ ID NO:46, respectively. In some embodiments, a B7-H7-binding agent (e.g., an antibody) comprises (a) a heavy chain variable region comprising: a heavy chain variable region CDR1 comprising TYTMH (SEQ ID NO: 122), a heavy chain variable region CDR2 comprising YINPSRGYSDYSQKFQG (SEQ ID NO:44), and a heavy chain variable region CDR3 comprising GGYDFDY (SEQ ID NO:45), and/or (b) a light chain variable region comprising: a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 122, SEQ ID NO:44, and SEQ ID NO:45, respectively; and (b) a light chain variable region comprising: a light chain variable region CDR1, CDR2, and CDR3 comprising the sequences of SEQ ID NO: 16, SEQ ID NO:30, and SEQ ID NO:46, respectively. In some embodiments, a B7-H7-binding agent (e.g., an antibody) comprises (a) a heavy chain variable region comprising: a heavy chain variable region CDR1 comprising TTYTMH (SEQ ID NO: 123), a heavy chain variable region CDR2 comprising WIGYINPSRGYSD (SEQ ID NO: 124), and a heavy chain variable region CDR3 comprising ARGGYDFD (SEQ ID NO: 125), and/or (b) a light chain variable region comprising: a light chain variable region CDR1 comprising GTAVAWY (SEQ ID NO:92), a light chain variable region CDR2 comprising LLLYWASTRH (SEQ ID NO: 126), and a light chain variable region CDR3 comprising QQHFITPY (SEQ ID NO: 127). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 123, SEQ ID NO: 124, and SEQ ID NO: 125, respectively; and (b) a light chain variable region comprising: a light chain variable region CDR1, CDR2, and CDR3 comprising the sequences of SEQ ID NO:92, SEQ ID NO: 126, and SEQ ID NO: 127, respectively.

In some embodiments, an anti-B7-H7 binding agent (e.g., antibody) comprises a heavy chain variable region comprising an amino acid sequence that has the three heavy chain variable region CDRs of antibody 16D5 and which has at least 75%, 80%, 85%, 90%, 95%, 96%, 97%, 98%, 99%, or 100% identity to the sequence of SEQ ID NO:47 and a light chain variable region comprising an amino acid sequence that has the three light chain variable region CDRs of antibody 16D5 and which has at least 75%, 80%, 85%, 90%, 95%, 96%, 97%, 98%, 99%, or 100% identity to the sequence of SEQ ID NO:48.

In some embodiments, a B7-H7-binding agent (e.g., an antibody) comprises a heavy chain variable region having at least about 80%, about 85%, about 90%, or about 95% sequence identity to SEQ ID NO:47 and/or a light chain variable region having at least about 80%, about 85%, about 90%, or about 95% sequence identity to SEQ ID NO:48. In some embodiments, a B7-H7-binding agent (e.g, an antibody) comprises a heavy chain variable region having at least about 80%, about 85%, about 90%, or about 95% sequence identity to SEQ ID NO:47. In some embodiments, a B7H7 binding agent (e.g., an antibody) comprises a light chain variable region having at least about 80%, about 85%, about 90%, or about 95% sequence identity to SEQ ID NO:48. In some embodiments, a B7-H7-binding agent (e.g., an antibody) comprises a heavy chain variable region having at least about 80%, about 85%, about 90%, or about 95% sequence identity to SEQ ID NO:47 and a light chain variable region having at least about 80%, about 85%, about 90%, or about 95% sequence identity to SEQ ID NO:48. In some embodiments, a B7-H7-binding agent comprises a heavy chain variable region comprising SEQ ID NO:47 and/or a light chain variable region comprising SEQ ID NO:48. In some embodiments, a B7-H7-binding agent comprises a heavy chain variable region comprising SEQ ID NO:47. In some embodiments, a B7-H7-binding agent comprises a light chain variable region comprising SEQ ID NO:48. In some embodiments, a B7-H7-binding agent comprises a heavy chain variable region of SEQ ID NO:47 and a light chain variable region of SEQ ID NO:48.

In some embodiments, a B7-H7-binding agent (e.g., an antibody) comprises (a) a heavy chain variable region CDR1 comprising GFSLTGYGVN (SEQ ID NO:49), a heavy chain variable region CDR2 comprising VIWGDGSTDYNSVLKS (SEQ ID NO:50), and a heavy chain variable region CDR3 comprising EATEYLYWYFDV (SEQ ID NO:51), and/or (b) a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, a B7-H7-binding agent comprises a heavy chain variable region CDR1 comprising GFSLTGYGVN (SEQ ID NO:49), a heavy chain variable region CDR2 comprising VIWGDGSTDYNSVLKS (SEQ ID NO:50), and a heavy chain variable region CDR3 comprising EATEYLYWYFDV (SEQ ID NO:51). In some embodiments, a B7-H7-binding agent comprises a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, a B7-H7-binding agent comprises (a) a heavy chain variable region CDR1 comprising GFSLTGYGVN (SEQ ID NO:49), a heavy chain variable region CDR2 comprising VIWGDGSTDYNSVLKS (SEQ ID NO:50), and a heavy chain variable region CDR3 comprising EATEYLYWYFDV (SEQ ID NO:51), and (b) a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, a B7-H7-binding agent comprises (a) a heavy chain variable region comprising a heavy chain variable region CDR1 comprising GFSLTGYGVN (SEQ ID NO:49), a heavy chain variable region CDR2 comprising VIWGDGSTDYNSVLKS (SEQ ID NO:50), and a heavy chain variable region CDR3 comprising EATEYLYWYFDV (SEQ ID NO:51), and (b) a light chain variable region comprising a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54).

In some embodiments, a B7-H7-binding agent (e.g., an antibody) comprises (a) a heavy chain variable region comprising: a heavy chain variable region CDR1 comprising GFSLTGY (SEQ ID NO: 128), a heavy chain variable region CDR2 comprising WGDGS (SEQ ID NO: 129), and a heavy chain variable region CDR3 comprising EATEYLYWYFDV (SEQ ID NO:51), and/or (b) a light chain variable region comprising: a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 128, SEQ ID NO: 129, and SEQ ID NO:51, respectively; and (b) a light chain variable region comprising: a light chain variable region CDR1, CDR2, and CDR3 comprising the sequences of SEQ ID NO:52, SEQ ID NO:53, and SEQ ID NO:54, respectively. In some embodiments, a B7-H7-binding agent (e.g., an antibody) comprises (a) a heavy chain variable region comprising: a heavy chain variable region CDR1 comprising GFSLTGYGVN (SEQ ID NO:49), a heavy chain variable region CDR2 comprising VIWGDGSTD (SEQ ID NO: 130), and a heavy chain variable region CDR3 comprising EATEYLYWYFDV (SEQ ID NO:51), and/or (b) a light chain variable region comprising: a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:49, SEQ ID NO: 130, and SEQ ID NO:51, respectively; and (b) a light chain variable region comprising: a light chain variable region CDR1, CDR2, and CDR3 comprising the sequences of SEQ ID NO:52, SEQ ID NO:53, and SEQ ID NO:54, respectively. In some embodiments, a B7-H7-binding agent (e.g., an antibody) comprises (a) a heavy chain variable region comprising: a heavy chain variable region CDR1 comprising GYGVN (SEQ ID NO: 131), a heavy chain variable region CDR2 comprising VIWGDGSTDYNSVLKS (SEQ ID NO:50), and a heavy chain variable region CDR3 comprising EATEYLYWYFDV (SEQ ID NO:51), and/or (b) a light chain variable region comprising: a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 131, SEQ ID NO:50, and SEQ ID NO:51, respectively; and (b) a light chain variable region comprising: a light chain variable region CDR1, CDR2, and CDR3 comprising the sequences of SEQ ID NO:52, SEQ ID NO:53, and SEQ ID NO:54, respectively. In some embodiments, a B7-H7-binding agent (e.g., an antibody) comprises (a) a heavy chain variable region comprising: a heavy chain variable region CDR1 comprising TGYGVN (SEQ ID NO: 132), a heavy chain variable region CDR2 comprising WLGVIWGDGSTD (SEQ ID NO: 133), and a heavy chain variable region CDR3 comprising AREATEYLYWYFD (SEQ ID NO: 134), and/or (b) a light chain variable region comprising: a light chain variable region CDR1 comprising EYYGSSLMQWY (SEQ ID NO: 135), a light chain variable region CDR2 comprising LLIFAASNVE (SEQ ID NO: 136), and a light chain variable region CDR3 comprising QQGRRVPW (SEQ ID NO: 137). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 132, SEQ ID NO: 133, and SEQ ID NO: 134, respectively; and (b) a light chain variable region comprising: a light chain variable region CDR1, CDR2, and CDR3 comprising the sequences of SEQ ID NO: 135, SEQ ID NO: 136, and SEQ ID NO: 137, respectively.

In some embodiments, an anti-B7-H7 binding agent (e.g., antibody) comprises a heavy chain variable region comprising an amino acid sequence that has the three heavy chain variable region CDRs of antibody 28F5 and winch has at least 75%, 80%, 85%, 90%, 95%, 96%, 97%, 98%, 99%, or 100% identity to the sequence of SEQ ID NO:55 and a light chain variable region comprising an amino acid sequence that has the three light chain variable region CDRs of antibody 28F5 and which has at least 75%, 80%, 85%, 90%, 95%, 96%, 97%, 98%, 99%, or 100% identity to the sequence of SEQ ID NO:56.

In some embodiments, a B7-H7-binding agent (e.g., an antibody) comprises a heavy chain variable region having at least about 80%, about 85%, about 90%, or about 95% sequence identity to SEQ ID NO:55 and/or a light chain variable region having at least about 80%, about 85%, about 90%, or about 95% sequence identity to SEQ ID NO:56. In some embodiments, a B7H7 binding agent (e.g., an antibody) comprises a heavy chain variable region having at least about 80%, about 85%, about 90%, or about 95% sequence identity to SEQ ID NO:55. In some embodiments, a B7-H7-binding agent (e.g., an antibody) comprises a light chain variable region having at least about 80%, about 85%, about 90%, or about 95% sequence identity to SEQ ID NO:56. In some embodiments, a B7-H7-binding agent (e.g., an antibody) comprises a heavy chain variable region having at least about 80%, about 85%, about 90%, or about 95% sequence identity to SEQ ID NO:55 and a light chain variable region having at least about 80%, about 85%, about 90%, or about 95% sequence identity to SEQ ID NO:56. In some embodiments, a B7-H7-binding agent comprises a heavy chain variable region comprising SEQ ID NO:55 and/or a light chain variable region comprising SEQ ID NO:56. In some embodiments, a B7-H7-binding agent comprises a heavy chain variable region comprising SEQ ID NO:55. In some embodiments, a B7-H7-binding agent comprises a light chain variable region comprising SEQ ID NO:56. In some embodiments, a B7-H7-binding agent comprises a heavy chain variable region of SEQ ID NO:55 and a light chain variable region of SEQ ID NO:56.

In some embodiments, a B7-H7-binding agent (e.g., an antibody) comprises (a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SVYDAPWLAH (SEQ ID NO:59), and/or (b) a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent comprises a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SVYDAPWLAH (SEQ ID NO:59). In some embodiments, a B7-H7-binding agent comprises a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent comprises (a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SVYDAPWLAH (SEQ ID NO:59), and (b) a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent comprises (a) a heavy chain variable region comprising a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SVYDAPWLAH (SEQ ID NO:59), and (b) a light chain variable region comprising a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62).

In some embodiments, a B7-H7-binding agent (e.g., an antibody) comprises (a) a heavy chain variable region comprising: a heavy chain variable region CDR1 comprising GYSFTGY (SEQ ID NO: 138), a heavy chain variable region CDR2 comprising DPYSGG (SEQ ID NO: 139), and a heavy chain variable region CDR3 comprising SVYDAPWLAH (SEQ ID NO:59), and/or (b) a light chain variable region comprising: a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 138, SEQ ID NO: 139, and SEQ ID NO:59, respectively; and (b) a light chain variable region comprising: a light chain variable region CDR1, CDR2, and CDR3 comprising the sequences of SEQ ID NO:60, SEQ ID NO:61, and SEQ ID NO:62, respectively. In some embodiments, a B7-H7-binding agent (e.g., an antibody) comprises (a) a heavy chain variable region comprising: a heavy chain variable region CDR1 comprising GYSFTGYNMN (SEQ ID NO:57), a heavy chain variable region CDR2 comprising NIDPYSGGST (SEQ ID NO: 140), and a heavy chain variable region CDR3 comprising SVYDAPWLAH (SEQ ID NO:59), and/or (b) a light chain variable region comprising: a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:57, SEQ ID NO: 140, and SEQ ID NO:59, respectively; and (b) a light chain variable region comprising: a light chain variable region CDR1, CDR2, and CDR3 comprising the sequences of SEQ ID NO:60, SEQ ID NO:61, and SEQ ID NO:62, respectively. In some embodiments, a B7-H7-binding agent (e.g., an antibody) comprises (a) a heavy chain variable region comprising: a heavy chain variable region CDR1 comprising GYNMN (SEQ ID NO: 141), a heavy chain variable region CDR2 comprising NIDPYSGGSTYNQKFKG (SEQ ID NO:58), and a heavy chain variable region CDR3 comprising SVYDAPWLAH (SEQ ID NO:59), and/or (b) a light chain variable region comprising: a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 141, SEQ ID NO:58, and SEQ ID NO:59, respectively; and (b) a light chain variable region comprising: a light chain variable region CDR1, CDR2, and CDR3 comprising the sequences of SEQ ID NO:60, SEQ ID NO:61, and SEQ ID NO:62, respectively. In some embodiments, a B7-H7-binding agent (e.g., an antibody) comprises (a) a heavy chain variable region comprising: a heavy chain variable region CDR1 comprising TGYNMN (SEQ ID NO: 142), a heavy chain variable region CDR2 comprising WIGNIDPYSGGST (SEQ ID NO: 143), and a heavy chain variable region CDR3 comprising ARSVYDAPWLA (SEQ ID NO: 144), and/or (b) a light chain variable region comprising: a light chain variable region CDR1 comprising YIYLAWY (SEQ ID NO: 145), a light chain variable region CDR2 comprising LLVYNAKTLA (SEQ ID NO: 146), and a light chain variable region CDR3 comprising QHHYGTPP (SEQ ID NO: 147). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 142, SEQ ID NO: 143, and SEQ ID NO: 144, respectively; and (b) a light chain variable region comprising: a light chain variable region CDR1, CDR2, and CDR3 comprising the sequences of SEQ ID NO: 145, SEQ ID NO: 146, and SEQ ID NO: 147, respectively.

In some embodiments, an anti-67H7 binding agent (e.g., antibody) comprises a heavy chain variable region comprising an amino acid sequence that has the three heavy chain variable region CDRs of antibody 1D3 and which has at least 75%, 80%, 85%, 90%, 95%, 96%, 97%, 98%, 99%, or 100% identity to the sequence of SEQ ID NO:63 and a light chain variable region comprising an amino acid sequence that has the three light chain variable region CDRs of antibody ID3 and which has at least 75%, 80%, 85%, 90%, 95%, 96%, 97%, 98%, 99%, or 100% identity to the sequence of SEQ ID NO:64.

In some embodiments, a B7-H7-binding agent (e.g., an antibody) comprises a heavy chain variable region having at least about 80%, about 85%, about 90%, or about 95% sequence identity to SEQ ID NO:63 and/or a light chain variable region having at least about 80%, about 85%, about 90%, or about 95% sequence identity to SEQ ID NO:64. In some embodiments, a 67H7 binding agent (e.g., an antibody) comprises a heavy chain variable region having at least about 80%, about 85%, about 90%, or about 95% sequence identity to SEQ ID NO:63. In some embodiments, a B7-H7-binding agent (e.g., an antibody) comprises a light chain variable region having at least about 80%, about 85%, about 90%, or about 95% sequence identity to SEQ ID NO:64. In some embodiments, a B7-H7-binding agent (e.g., an antibody) comprises a heavy chain variable region having at least about 80%, about 85%, about 90%, or about 95% sequence identity to SEQ ID NO:63 and a light chain variable region having at least about 80%, about 85%, about 90%, or about 95% sequence identity to SEQ ID NO:64. In some embodiments, a B7-H7-binding agent comprises a heavy chain variable region comprising SEQ ID NO:63 and/or a light chain variable region comprising SEQ ID NO:64. In some embodiments, a B7-H7-binding agent comprises a heavy chain variable region comprising SEQ ID NO:63. In some embodiments, a B7-H7-binding agent comprises a light chain variable region comprising SEQ ID NO:64. In some embodiments, a B7-H7-binding agent comprises a heavy chain variable region of SEQ ID NO:63 and a light chain variable region of SEQ ID NO:64.

In some embodiments, a B7-H7-binding agent (e.g., an antibody) comprises (a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SFYDAPYLTY (SEQ ID NO:65), and/or (b) a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent comprises a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SFYDAPYLTY (SEQ ID NO:65). In some embodiments, a B7-H7-binding agent comprises a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent comprises (a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SFYDAPYLTY (SEQ ID NO:65), and (b) a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent comprises (a) a heavy chain variable region comprising a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SFYDAPYLTY (SEQ ID NO:65), and (b) a light chain variable region comprising a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62).

In some embodiments, a B7-H7-binding agent (e.g., an antibody) comprises (a) a heavy chain variable region comprising: a heavy chain variable region CDR1 comprising GYSFTGY (SEQ ID NO: 138), a heavy chain variable region CDR2 comprising DPYSGG (SEQ ID NO: 139), and a heavy chain variable region CDR3 comprising SFYDAPYLTY (SEQ ID NO:65), and/or (b) a light chain variable region comprising: a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 138, SEQ ID NO: 139, and SEQ ID NO:65, respectively; and (b) a light chain variable region comprising: a light chain variable region CDR1, CDR2, and CDR3 comprising the sequences of SEQ ID NO:66, SEQ ID NO:61, and SEQ ID NO:62, respectively. In some embodiments, a B7-H7-binding agent (e.g., an antibody) comprises (a) a heavy chain variable region comprising: a heavy chain variable region CDR1 comprising GYSFTGYNMN (SEQ ID NO:57), a heavy chain variable region CDR2 comprising NIDPYSGGST (SEQ ID NO: 140), and a heavy chain variable region CDR3 comprising SFYDAPYLTY (SEQ ID NO:65), and/or (b) a light chain variable region comprising: a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:57, SEQ ID NO: 140, and SEQ ID NO:65, respectively; and (b) a light chain variable region comprising: a light chain variable region CDR1, CDR2, and CDR3 comprising the sequences of SEQ ID NO:66, SEQ ID NO:61, and SEQ ID NO:62, respectively. In some embodiments, a B7-H7-binding agent (e.g., an antibody) comprises (a) a heavy chain variable region comprising: a heavy chain variable region CDR1 comprising GYNMN (SEQ ID NO: 141), a heavy chain variable region CDR2 comprising NIDPYSGGSTYNQKFKG (SEQ ID NO:58), and a heavy chain variable region CDR3 comprising SFYDAPYLTY (SEQ ID NO:65), and/or (b) a light chain variable region comprising: a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 141, SEQ ID NO:58, and SEQ ID NO:65, respectively; and (b) a light chain variable region comprising: a light chain variable region CDR1, CDR2, and CDR3 comprising the sequences of SEQ ID NO:66, SEQ ID NO:61, and SEQ ID NO:62, respectively. In some embodiments, a B7-H7-binding agent (e.g., an antibody) comprises (a) a heavy chain variable region comprising: a heavy chain variable region CDR1 comprising TGYNMN (SEQ ID NO: 142), a heavy chain variable region CDR2 comprising WIGNIDPYSGGST (SEQ ID NO: 143), and a heavy chain variable region CDR3 comprising ARSFYDAPYLT (SEQ ID NO: 148), and/or (b) a light chain variable region comprising: a light chain variable region CDR1 comprising FIYLAWY (SEQ ID NO: 149), a light chain variable region CDR2 comprising LLVFNAKTLA (SEQ ID NO: 150), and a light chain variable region CDR3 comprising QHHYGTPP (SEQ ID NO: 147). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 142, SEQ ID NO: 143, and SEQ ID NO: 148, respectively; and (b) a light chain variable region comprising: a light chain variable region CDR1, CDR2, and CDR3 comprising the sequences of SEQ ID NO: 149, SEQ ID NO: 150, and SEQ ID NO: 147, respectively.

In some embodiments, an anti-B7-H7 binding agent (e.g., antibody) comprises a heavy chain variable region comprising an amino acid sequence that has the three heavy chain variable region CDRs of antibody 5B10 and which has at least 75%, 80%, 85%, 90%, 95%, 96%, 97%, 98%, 99%, or 100% identity to the sequence of SEQ ID NO:67 and a light chain variable region comprising an amino acid sequence that has the three light chain variable region CDRs of antibody 5B10 and which has at least 75%, 80%, 85%, 90%, 95%, 96%, 97%, 98%, 99%, or 100% identity to the sequence of SEQ ID NO:68.

In some embodiments, a B7-H7-binding agent (e.g., an antibody) comprises a heavy chain variable region having at least about 80%, about 85%, about 90%, or about 95% sequence identity to SEQ ID NO:67 and/or a light chain variable region having at least about 80%, about 85%, about 90%, or about 95% sequence identity to SEQ ID NO:68. In some embodiments, a B7-H7-binding agent (e.g, an antibody) comprises a heavy chain variable region having at least about 80%, about 85%, about 90%, or about 95% sequence identity to SEQ ID NO:67. In some embodiments, a B7-H7-binding agent (e.g., an antibody) comprises a light chain variable region having at least about 80%, about 85%, about 90%, or about 95% sequence identity to SEQ ID NO:68. In some embodiments, a B7-H7-binding agent (e.g., an antibody) comprises a heavy chain variable region having at least about 80%, about 85%, about 90%, or about 95% sequence identity to SEQ ID NO:67 and a light chain variable region having at least about 80%, about 85%, about 90%, or about 95% sequence identity to SEQ ID NO:68. In some embodiments, a B7-H7-binding agent comprises a heavy chain variable region comprising SEQ ID NO:67 and/or a light chain variable region comprising SEQ ID NO:68. In some embodiments, a B7-H7-binding agent comprises a heavy chain variable region comprising SEQ ID NO:67. In some embodiments, a B7-H7-binding agent comprises a light chain variable region comprising SEQ ID NO:68. In some embodiments, a B7-H7-binding agent comprises a heavy chain variable region of SEQ ID NO:67 and a light chain variable region of SEQ ID NO:68.

In some embodiments, a B7-H7-binding agent (e.g., an antibody) comprises (a) a heavy chain variable region CDR1 comprising GYTFTSFWIH (SEQ ID NO:69), a heavy chain variable region CDR2 comprising YIIPNTDYTEYNQKFKD (SEQ ID NO:70), and a heavy chain variable region CDR3 comprising GLRGAYYFDY (SEQ ID NO:71), and/or (b) a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74). In some embodiments, a B7-H7-binding agent comprises a heavy chain variable region CDR1 comprising GYTFTSFWIH (SEQ ID NO:69), a heavy chain variable region CDR2 comprising YIIPNTDYTEYNQKFKD (SEQ ID NO:70), and a heavy chain variable region CDR3 comprising GLRGAYYFDY (SEQ ID NO:71). In some embodiments, a B7-H7-binding agent comprises a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74). In some embodiments, a B7-H7-binding agent comprises (a) a heavy chain variable region CDR1 comprising GYTFTSFWIH (SEQ ID NO:69), a heavy chain variable region CDR2 comprising YIIPNTDYTEYNQKFKD (SEQ ID NO:70), and a heavy chain variable region CDR3 comprising GLRGAYYFDY (SEQ ID NO:71), and (b) a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74). In some embodiments, a B7-H7-binding agent comprises (a) a heavy chain variable region comprising a heavy chain variable region CDR1 comprising GYTFTSFWIH (SEQ ID NO:69), a heavy chain variable region CDR2 comprising YIIPNTDYTEYNQKFKD (SEQ ID NO:70), and a heavy chain variable region CDR3 comprising GLRGAYYFDY (SEQ ID NO:71), and (b) a light chain variable region comprising a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74).

In some embodiments, a B7-H7-binding agent (e.g., an antibody) comprises (a) a heavy chain variable region comprising: a heavy chain variable region CDR1 comprising GYTFTSF (SEQ ID NO: 151), a heavy chain variable region CDR2 comprising IPNTDY (SEQ ID NO: 152), and a heavy chain variable region CDR3 comprising GLRGAYYFDY (SEQ ID NO:71), and/or (b) a light chain variable region comprising: a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 151, SEQ ID NO: 152, and SEQ ID NO:71, respectively; and (b) a light chain variable region comprising: a light chain variable region CDR1, CDR2, and CDR3 comprising the sequences of SEQ ID NO:72, SEQ ID NO:73, and SEQ ID NO:74, respectively. In some embodiments, a B7-H7-binding agent (e.g., an antibody) comprises (a) a heavy chain variable region comprising: a heavy chain variable region CDR1 comprising GYTFTSFWIH (SEQ ID NO:69), a heavy chain variable region CDR2 comprising YIIPNTDYTE (SEQ ID NO: 153), and a heavy chain variable region CDR3 comprising GLRGAYYFDY (SEQ ID NO:71), and/or (b) a light chain variable region comprising: a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO:69, SEQ ID NO: 153, and SEQ ID NO:71, respectively; and (b) a light chain variable region comprising: a light chain variable region CDR1, CDR2, and CDR3 comprising the sequences of SEQ ID NO:72, SEQ ID NO:73, and SEQ ID NO:74, respectively. In some embodiments, a B7-H7-binding agent (e.g., an antibody) comprises (a) a heavy chain variable region comprising: a heavy chain variable region CDR1 comprising SFWIH (SEQ ID NO: 154), a heavy chain variable region CDR2 comprising YIIPNTDYTEYNQKFKD (SEQ ID NO:70), and a heavy chain variable region CDR3 comprising GLRGAYYFDY (SEQ ID NO:71), and/or (b) a light chain variable region comprising: a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 154, SEQ ID NO:70, and SEQ ID NO:71, respectively; and (b) a light chain variable region comprising: a light chain variable region CDR1, CDR2, and CDR3 comprising the sequences of SEQ ID NO:72, SEQ ID NO:73, and SEQ ID NO:74, respectively. In some embodiments, a B7-H7-binding agent (e.g., an antibody) comprises (a) a heavy chain variable region comprising: a heavy chain variable region CDR1 comprising TSFWIH (SEQ ID NO: 155), a heavy chain variable region CDR2 comprising WIGYIIPNTDYTE (SEQ ID NO: 156), and a heavy chain variable region CDR3 comprising ARGLRGAYYFD (SEQ ID NO: 157), and/or (b) a light chain variable region comprising: a light chain variable region CDR1 comprising STSTNGYMHWY (SEQ ID NO: 158), a light chain variable region CDR2 comprising LLIMYASNLE (SEQ ID NO: 159), and a light chain variable region CDR3 comprising QHSWVLPY (SEQ ID NO: 160). In some embodiments, a B7-H7-binding agent (e.g., an antibody) comprises: (a) a heavy chain variable region comprising: a heavy chain variable region CDR1, CDR2, and CDR3 comprising the sequences of SEQ ID NO: 155, SEQ ID NO: 156, and SEQ ID NO: 157, respectively; and (b) a light chain variable region comprising: a light chain variable region CDR1, CDR2, and CDR3 comprising the sequences of SEQ ID NO: 158, SEQ ID NO: 159, and SEQ ID NO: 160, respectively.

In some embodiments, an anti-B7-H7 binding agent (e.g., antibody) comprises a heavy chain variable region comprising an amino acid sequence that has the three heavy chain variable region CDRs of antibody 5C9 and which has at least 75%, 80%, 85%, 90%, 95%, 96%, 97%, 98%, 99%, or 100% identity to the sequence of SEQ ID NO:75 and a light chain variable region comprising an amino acid sequence that has the three light chain variable region CDRs of antibody 5C9 and which has at least 75%, 80%, 85%, 90%, 95%, 96%, 97%, 98%, 99%, or 100% identity to the sequence of SEQ ID NO:76.

In some embodiments, a B7-H7-binding agent (e.g., an antibody) comprises a heavy chain variable region having at least about 80%, about 85%, about 90%, or about 95% sequence identity to SEQ ID NO:75 and/or a light chain variable region having at least about 80%, about 85%, about 90%, or about 95% sequence identity to SEQ ID NO:76. In some embodiments, a B7-H7-binding agent (e.g, an antibody) comprises a heavy chain variable region having at least about 80%, about 85%, about 90%, or about 95% sequence identity to SEQ ID NO:75. In some embodiments, a B7-H7-binding agent (e.g., an antibody) comprises a light chain variable region having at least about 80%, about 85%, about 90%, or about 95% sequence identity to SEQ ID NO:76. In some embodiments, a B7-H7-binding agent (e.g., an antibody) comprises a heavy chain variable region having at least about 80%, about 85%, about 90%, or about 95% sequence identity to SEQ ID NO:75 and a light chain variable region having at least about 80%, about 85%, about 90%, or about 95% sequence identity to SEQ ID NO:76, In some embodiments, a B7-H7-binding agent comprises a heavy chain variable region comprising SEQ ID NO:75 and/or a light chain variable region comprising SEQ ID NO:76. In some embodiments, a B7-H7-binding agent comprises a heavy chain variable region comprising SEQ ID NO:75. In some embodiments, a B7-H7-binding agent comprises a light chain variable region comprising SEQ ID NO:76. In some embodiments, a B7-H7-binding agent comprises a heavy chain variable region of SEQ ID NO:75 and a light chain variable region of SEQ ID NO:76.

Provided herein are agents that compete with one or more of the antibodies described herein for binding to human B7-H7. In some embodiments, an agent that competes with one or more of the antibodies described herein for binding to human B7-H7 is an antibody. In some embodiments, an antibody binds the same epitope as one of the anti-B7-H7 antibodies described herein. In some embodiments, an antibody binds an epitope overlapping with an epitope bound by one of the anti-B7-H7 antibodies described herein. In some embodiments, an antibody that competes with one or more of the antibodies described herein for binding to B7-H7 is identified using a epitope binning method as described herein. Antibodies and antigen-binding fragments that compete with, or bind to the same epitope, as the anti-B7-H7 antibodies described herein are expected to show similar functional properties.

In some embodiments, an antibody competes for binding to B7-H7 with a B7-H7-binding agent (e.g., an antibody) described herein. In some embodiments, an antibody competes for binding to human B7-H7 with a B7-H7-binding agent (e.g., an antibody) described herein. In some embodiments, an antibody competes for binding to B7-H7 (e.g., human B7-H7) with a reference antibody, wherein the reference antibody comprises: (a) a heavy chain variable region comprising a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15); and (b) a light chain variable region comprising a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18). In some embodiments, an antibody competes for binding to B7-H7 (e.g., human B7-H7) with a reference antibody, wherein the reference antibody comprises a heavy chain variable region comprising SEQ ID NO: 19 and a light chain variable region comprising SEQ ID NO:20. In some embodiments, an antibody competes for binding to B7-H7 (e.g., human B7-H7) with a reference antibody, wherein the reference antibody comprises a heavy chain variable region comprising SEQ ID NO:21 and a light chain variable region comprising SEQ ID NO:22.

In some embodiments, an antibody competes for binding to B7-H7 (e.g., human B7-H7) with a reference antibody, wherein the reference antibody comprises: (a) a heavy chain variable region comprising a heavy chain variable region CDR1 comprising GYSFTDYIIV (SEQ ID NO:27), a heavy chain variable region CDR2 comprising KINPYYGTTTYNLRFED (SEQ ID NO:28), and a heavy chain variable region CDR3 comprising WDYVSTLFAMDY (SEQ ID NO:29); and (b) a light chain variable region comprising a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31). In some embodiments, an antibody competes for binding to B7-H7 (e.g., human B7-H7) with a reference antibody, wherein the reference antibody comprises a heavy chain variable region comprising SEQ ID NO:32 and a light chain variable region comprising SEQ ID NO:33.

In some embodiments, an antibody competes for binding to B7-H7 (e.g, human B7-H7) with a reference antibody, wherein the reference antibody comprises: (a) a heavy chain variable region comprising a heavy chain variable region CDR1 comprising GYTFTEYTMH (SEQ ID NO: 13), a heavy chain variable region CDR2 comprising GINPNNGGAPYNQKFKG (SEQ ID NO:34), and a heavy chain variable region CDR3 comprising GGYYFDY (SEQ ID NO: 15); and (b) a light chain variable region comprising a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18). In some embodiments, an antibody competes for binding to B7-H7 (e.g, human B7-H7) with a reference antibody, wherein the reference antibody comprises a heavy chain variable region comprising SEQ ID NO:36 and a light chain variable region comprising SEQ ID NO:37.

In some embodiments, an antibody competes for binding to B7-H7 (e.g, human B7-H7) with a reference antibody, wherein the reference antibody comprises: (a) a heavy chain variable region comprising a heavy chain variable region CDR1 comprising GYTFSSYSMH (SEQ ID NO:38), a heavy chain variable region CDR2 comprising TIYPGNENTSYNQKFKG (SEQ ID NO:39), and a heavy chain variable region CDR3 comprising GGYYFDY (SEQ ID NO: 15); and (b) a light chain variable region comprising a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40). In some embodiments, an antibody competes for binding to B7-H7 (e.g, human B7-H7) with a reference antibody, wherein the reference antibody comprises a heavy chain variable region comprising SEQ ID NO:41 and a light chain variable region comprising SEQ ID NO:42.

In some embodiments, an antibody competes for binding to B7-H7 (e.g, human B7-H7) with a reference antibody, wherein the reference antibody comprises: (a) a heavy chain variable region comprising a heavy chain variable region CDR1 comprising GYTFTTYTMH (SEQ ID NO:43), a heavy chain variable region CDR2 comprising YINPSRGYSDYSQKFQG (SEQ ID NO:44), and a heavy chain variable region CDR3 comprising GGYDFDY (SEQ ID NO:45); and (b) a light chain variable region comprising a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46). In some embodiments, an antibody competes for binding to B7-H7 (e.g., human B7-H7) with a reference antibody, wherein the reference antibody comprises a heavy chain variable region comprising SEQ ID NO:47 and a light chain variable region comprising SEQ ID NO:48.

In some embodiments, an antibody competes for binding to B7-H7 (e.g., human B7-H7) with a reference antibody, wherein the reference antibody comprises: (a) a heavy chain variable region comprising a heavy chain variable region CDR1 comprising GFSLTGYGVN (SEQ ID NO:49), a heavy chain variable region CDR2 comprising VIWGDGSTDYNSVLKS (SEQ ID NO:50), and a heavy chain variable region CDR3 comprising EATEYLYWYFDV (SEQ ID NO:51); and (b) a light chain variable region comprising a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54). In some embodiments, an antibody competes for binding to B7-H7 (e.g., human B7-H7) with a reference antibody, wherein the reference antibody comprises a heavy chain variable region comprising SEQ ID NO:55 and a light chain variable region comprising SEQ ID NO:56.

In some embodiments, an antibody competes for binding to B7-H7 (e.g., human B7-H7) with a reference antibody, wherein the reference antibody comprises: (a) a heavy chain variable region comprising a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SVYDAPWLAH (SEQ ID NO:59); and (b) a light chain variable region comprising a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62). In some embodiments, an antibody competes for binding to B7-H7 (e.g., human B7-H7) with a reference antibody, wherein the reference antibody comprises a heavy chain variable region comprising SEQ ID NO:63 and a light chain variable region comprising SEQ ID NO:64.

In some embodiments, an antibody competes for binding to B7-H7 (e.g., human B7-H7) with a reference antibody, wherein the reference antibody comprises: (a) a heavy chain variable region comprising a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SFYDAPYLTY (SEQ ID NO:65); and (b) a light chain variable region comprising a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62). In some embodiments, an antibody competes for binding to B7-H7 (e.g., human B7-H7) with a reference antibody, wherein the reference antibody comprises a heavy chain variable region comprising SEQ ID NO:67 and a light chain variable region comprising SEQ ID NO:68.

In some embodiments, an antibody competes for binding to B7-H7 (e.g., human B7-H7) with a reference antibody, wherein the reference antibody comprises: (a) a heavy chain variable region comprising a heavy chain variable region CDR1 comprising GYTFTSFWIH (SEQ ID NO:69), a heavy chain variable region CDR2 comprising YIIPNTDYTEYNQKFKD (SEQ ID NO:70), and a heavy chain variable region CDR3 comprising GLRGAYYFDY (SEQ ID NO:71); and (b) a light chain variable region comprising a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74). In some embodiments, an antibody competes for binding to B7-H7 (e.g, human B7-H7) with a reference antibody, wherein the reference antibody comprises a heavy chain variable region comprising SEQ ID NO:75 and a light chain variable region comprising SEQ ID NO:76.

In some embodiments, a B7-H7-binding agent described herein comprises an antibody in which at least one or more of the constant regions has been modified or deleted. In some embodiments, an antibody may comprise one or more modifications to one or more of the three heavy chain constant regions (CH1, CH2 or CH3) and/or to the light chain constant region (CL). In some embodiments, an antibody may comprise one or more modifications to the hinge region. In some embodiments, the heavy chain constant region of the modified antibody comprises at least one human constant region. In some embodiments, the heavy chain constant region of the modified antibody comprises more than one human constant region. In some embodiments, modifications to the constant region comprise additions, deletions, or substitutions of one or more amino acids in one or more regions. In some embodiments, one or more regions are partially or entirely deleted from the constant regions of a modified antibody. In some embodiments, one or more regions are partially or entirely deleted from the hinge region of a modified antibody. In some embodiments, the entire CH2 domain has been removed from an antibody. In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

It is known in the art that the constant region(s) of an antibody mediates several effector functions and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors that are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, an antibody comprises a variant Fc region. The amino acid sequences of the Fc region of human IgG1, IgG2, IgG3, and IgG4 are known to those of ordinary skill in the art (e.g., a representative human IgG1 is SEQ ID NO:77). In some cases, Fc regions with amino acid variations have been identified in native antibodies. In some embodiments, a variant Fc region is engineered with substitutions at specific amino acid positions as compared to a native Fc region (e.g, SEQ ID NO:78, SEQ ID NO:79, and SEQ ID NO:80).

In some embodiments, a modified antibody (e.g., comprising a modified Fc region) provides for altered effector functions that, in turn, affect the biological profile of the antibody. For example, in some embodiments, the deletion or inactivation (through point mutations or other means) of a constant region reduces Fc receptor binding of a modified antibody as it circulates. In some embodiments, the constant region modifications increase the serum half-life of an antibody. In some embodiments, the constant region modifications reduce the serum half-life of an antibody. In some embodiments, the constant region modifications decrease or remove ADCC and/or complement dependent cytotoxicity (CDC) of an antibody. In some embodiments, specific amino acid substitutions in a human IgG1 Fc region with corresponding IgG2 or IgG4 residues may reduce effector functions (e.g., ADCC and CDC) in a modified antibody. In some embodiments, the constant region modifications decrease or reduce ADCC and/or CDC of an antibody. In some embodiments, an antibody does not have one or more effector functions (e.g., “effectorless” antibodies). In some embodiments, an antibody has no ADCC activity and/or no CDC activity. In some embodiments, an antibody does not bind an Fc receptor and/or complement factors. In some embodiments, an antibody has no effector function(s). In some embodiments, the constant region modifications increase or enhance ADCC and/or CDC of an antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites.

In some embodiments, a B7-H7-binding agent comprises a heavy chain having at least 80%, at least 85%, at least 90%, or at least 95% sequence identity to SEQ ID NO:24. In some embodiments, a B7-H7-binding agent comprises a light chain having at least 80%, at least 85%, at least 90%, or at least 95% sequence identity to SEQ ID NO:26. In some embodiments, a 67H7 binding agent comprises a heavy chain having at least 80%, at least 85%, at least 90%, or at least 95% sequence identity to SEQ ID NO:24 and a light chain having at least 80%, at least 85%, at least 90%, or at least 95% sequence identity to SEQ ID NO:26. In some embodiments, a B7-H7-binding agent comprises a heavy chain having at least 90% sequence identity to SEQ ID NO:24. In some embodiments, a B7-H7-binding agent comprises a light chain having at least 90% sequence identity to SEQ ID NO:26. In some embodiments, a B7-H7-binding agent comprises a heavy chain having at least 90% sequence identity to SEQ ID NO:24 and a light chain having at least 90% sequence identity to SEQ ID NO:26. In some embodiments, a B7-H7-binding agent comprises a heavy chain comprising the amino acid sequence of SEQ ID NO:24. In some embodiments, a B7-H7-binding agent comprises a light chain comprising the amino acid sequence of SEQ ID NO:26. In some embodiments, a B7-H7-binding agent comprises a heavy chain comprising the amino acid sequence of SEQ ID NO:24 and a light chain comprising the amino acid sequence of SEQ ID NO:26, In some embodiments, a B7-H7-binding agent is an antibody that comprises a heavy chain of SEQ ID NO:24 and/or a light chain of SEQ ID NO:26. In some embodiments, a B7-H7-binding agent is an antibody that comprises a heavy chain of SEQ ID NO:24. In some embodiments, a B7-H7-binding agent is an antibody that comprises a light chain of SEQ ID NO:26. In some embodiments, a B7-H7-binding agent is an antibody that comprises a heavy chain of SEQ ID NO:24 and a light chain of SEQ ID NO:26.

Modifications to the constant region of antibodies described herein may be made using well-known biochemical or molecular engineering techniques. In some embodiments, antibody variants are prepared by introducing appropriate nucleotide changes into the encoding DNA, and/or by synthesis of the desired antibody or polypeptide. Using this technique, it may be possible to disrupt the activity or effector function provided by a specific sequence or region while substantially maintaining the structure, binding activity, and other desired characteristics of the modified antibody.

The present disclosure further embraces additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, for example, conservative amino acid replacements. Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant may be determined by systematically making insertions, deletions, or substitutions in the sequence and testing the resulting variant proteins for activity as compared to the parent protein.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g, a fluorescent tag or an enzyme).

In some embodiments, a cysteine residue not involved in maintaining the proper conformation of an antibody is substituted or deleted to modulate the antibody's characteristics, for example, to improve oxidative stability and/or prevent aberrant disulfide crosslinking. Conversely, in some embodiments, one or more cysteine residues are added to create disulfide bond(s) to improve stability.

In some embodiments, an antibody of the present disclosure is “deimmunized”. The deimmunization of antibodies generally consists of introducing specific amino acid mutations (e.g., substitutions, deletions, additions) that result in removal of predicted T-cell epitopes without significantly reducing the binding affinity or other desired characteristics of the antibody.

The variant antibodies or polypeptides described herein may be generated using methods known in the art, including but not limited to, site-directed mutagenesis, alanine scanning mutagenesis, and PCR mutagenesis.

In some embodiments, a B7-H7-binding agent described herein is chemically modified. In some embodiments, a B7-H7-binding agent is an anti-B7-H7 antibody that has been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques.

The present disclosure encompasses B7-H7-binding agents built upon non-immunoglobulin backbones, wherein the agents bind the same epitope or essentially the same epitope as an anti-B7-H7 antibody disclosed herein. In some embodiments, a non-immunoglobulin-based binding agent is an agent that competes with an anti-B7-H7 antibody described herein in a competitive binding assay. In some embodiments, an alternative B7-H7-binding agent comprises a scaffold protein. Generally, scaffold proteins can be assigned to one of three groups based on the architecture of their backbone (1) scaffolds consisting of α-helices; (2) small scaffolds with few secondary structures or an irregular architecture of α-helices and β-sheets; and (3) scaffolds consisting of predominantly β-sheets. Scaffold proteins include, but are not limited to, anticalins, which are based upon the lipocalin scaffold; adnectins, which are based on the 10^{th} domain of human fibronectin type 3; affibodies, which are based on the B-domain in the Ig-binding region of Staphylococcus aureus protein A; darpins, which are based on ankyrin repeat domain proteins; fynomers, which are based on the SH3 domain of the human Fyn protein kinase; affitins, which are based on Sac7d from Sulfolobus acidocaldarius; affilins, which are based on human γ-B-crystallin or human ubiquitin; avimers, which are based on the A-domains of membrane receptor proteins; knottins (cysteine knot miniproteins), which are based upon a stable 30-amino acid anti-parallel β-strand protein fold; and Kunitz domain inhibitor scaffolds, which are based upon a structure that contains three disulfide bonds and three loops.

In some embodiments, a B7-H7-binding agent comprises an engineered scaffold protein comprising a heavy chain variable region CDR1, CDR2, and CDR3 and a light chain variable region CDR1, CDR2, and CDR3 from antibody 2D7. In some embodiments, a B7-H7-binding agent comprises an engineered scaffold protein comprising a heavy chain variable region CDR1, CDR2, and CDR3 and a light chain variable region CDR1, CDR2, and CDR3 from antibody 2F10. In some embodiments, a B7-H7-binding agent comprises an engineered scaffold protein comprising a heavy chain variable region CDR1, CDR2, and CDR3 and a light chain variable region CDR1, CDR2, and CDR3 from antibody 3H6. In some embodiments, a B7-H7-binding agent comprises an engineered scaffold protein comprising a heavy chain variable region CDR1, CDR2, and CDR3 and a light chain variable region CDR1, CDR2, and CDR3 from antibody 5F7. In some embodiments, a B7-H7-binding agent comprises an engineered scaffold protein comprising a heavy chain variable region CDR1, CDR2, and CDR3 and a light chain variable region CDR1, CDR2, and CDR3 from antibody 16D5. In some embodiments, a B7-H7-binding agent comprises an engineered scaffold protein comprising a heavy chain variable region CDR1, CDR2, and CDR3 and a light chain variable region CDR1, CDR2, and CDR3 from antibody 28F5. In some embodiments, a B7-H7-binding agent comprises an engineered scaffold protein comprising a heavy chain variable region CDR1, CDR2, and CDR3 and a light chain variable region CDR1, CDR2, and CDR3 from antibody 1D3. In some embodiments, a B7-H7-binding agent comprises an engineered scaffold protein comprising a heavy chain variable region CDR1, CDR2, and CDR3 and a light chain variable region CDR1, CDR2, and CDR3 from antibody 5B10. In some embodiments, a B7-H7-binding agent comprises an engineered scaffold protein comprising a heavy chain variable region CDR1, CDR2, and CDR3 and a light chain variable region CDR1, CDR2, and CDR3 from antibody 5C9.

Generally speaking, antigen-antibody interactions are non-covalent and reversible, formed by a combination of hydrogen bonds, hydrophobic interactions, electrostatic and van der Waals forces. When describing the strength of an antigen-antibody complex, the terms affinity and/or avidity are often used. The binding of an antibody to its antigen is a reversible process, and the affinity of the binding is typically reported as an equilibrium dissociation constant (K_{D}). K_{D} is the ratio of an antibody dissociation rate (k_{off}) (how quickly it dissociates from its antigen) to the antibody association rate (kₒₙ) (how quickly it binds to its antigen). In some embodiments, K_{D} values are determined by measuring the kₒₙ and k_{off} rates of a specific antibody/antigen interaction and then using a ratio of these values to calculate the K_{D} value. In some embodiments, K_{D} values are used to evaluate and rank the strength of individual antibody/antigen interactions. The lower the K_{D} of an antibody, the higher the affinity of the antibody for its target. In some embodiments, affinity is measured using SPR technology in a Biacore system. Avidity gives a measure of the overall strength of an antibody-antigen complex. It is dependent on three major parameters: (i) affinity of the antibody for the target, (ii) valency of both the antibody and antigen, and (iii) structural arrangement of the parts that interact.

In some embodiments, a B7-H7-binding agent (e.g., an antibody) binds B7-H7 with a K_{D} of about 1 μM or less, about 100 nM or less, about 40 nM or less, about 20 nM or less, about 10 nM or less, about 1 nM or less, about 0.1 nM or less, 50 pM or less, 10 pM or less, or 1 pM or less. In some embodiments, a B7-H7-binding agent binds B7-H7 with a K_{D} of about 20 nM or less. In some embodiments, a B7-H7-binding agent binds B7-H7 with a K_{D} of about 10 nM or less. In some embodiments, a B7-H7-binding agent binds B7-H7 with a K_{D} of about 5 nM or less. In some embodiments, a B7-H7-binding agent binds B7-H7 with a K_{D} of about 3 nM or less. In some embodiments, a B7-H7-binding agent binds B7-H7 with a K_{D} of about 2 nM or less. In some embodiments, a B7-H7-binding agent binds B7-H7 with a K_{D} of about 1 nM or less. In some embodiments, a B7-H7-binding agent binds B7-H7 with a K_{D} of about 0.5 nM or less. In some embodiments, a B7-H7-binding agent binds B7-H7 with a K_{D} of about 0.1 nM or less. In some embodiments, a B7-H7-binding agent binds B7-H7 with a K_{D} of about 50 pM or less. In some embodiments, a B7-H7-binding agent binds B7-H7 with a K_{D} of about 25 pM or less. In some embodiments, a B7-H7-binding agent binds B7-H7 with a K_{D} of about 10 pM or less. In some embodiments, a B7-H7-binding agent binds B7-H7 with a K_{D} of about 1 pM or less. In some embodiments, a B7-H7-binding agent binds B7-H7 with a K_{D} of about 0.01 nM to about 2.5 nM. In some embodiments, a B7-H7-binding agent binds B7-H7 with a K_{D} of about 0.1 nM to about 5 nM. In some embodiments, a B7-H7-binding agent binds B7-H7 with a K_{D} of about 1 nM to about 5 nM. In some embodiments, the dissociation constant of the binding agent (e.g., an antibody) to B7-H7 is the dissociation constant determined using a B7-H7 protein or a fragment thereof immobilized on a Biacore chip with the binding agent flowed over the chip. In some embodiments, the dissociation constant of the binding agent (e.g., an antibody) for B7-H7 is the dissociation constant determined using the binding agent captured on a Biacore chip with soluble B7-H7 flowed over the chip.

In some embodiments, a B7-H7-binding agent (e.g., an antibody) binds B7-H7 with a half maximal effective concentration (EC50) of about 1 pM or less, about 100 nM or less, about 40 nM or less, about 20 nM or less, about 10 nM or less, about 1 nM or less, or about 0.1 nM or less. In some embodiments, a B7-H7-binding agent binds to human B7-H7 with an EC50 of about 1 pM or less, about 100 nM or less, about 40 nM or less, about 20 nM or less, about 10 nM or less, about 1 nM or less, or about 0.1 nM or less. In some embodiments, a B7-H7-binding agent binds cyno B7-H7 and/or human B7-H7 with an EC50 of about 40 nM or less, about 20 nM or less, about 10 nM or less, about 1 nM or less or about 0.1 nM or less. In some embodiments, a B7-H7-binding agent binds B7-H7 with an EC50 of 0.1 nM to 3 nM, 0.1 nM to 2 nM, 0.1 nM to 1 nM, 0.5 nM to 3 nM, 0.5 nM to 2 nM, or 0.5 nM to 1 nM.

The B7-H7-binding agents (e.g., antibodies) described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest is constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), a polynucleotide sequence encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In some embodiments, a recombinant expression vector is used to amplify and express DNA encoding an antibody against human B7-H7. For example, a recombinant expression vector can be a replicable DNA construct that includes synthetic or cDNA-derived DNA fragments encoding a polypeptide chain of a B7-H7-binding agent, such as an anti-B7-H7 antibody operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence that is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can also be included. DNA regions are “operatively linked” when they are functionally related to each other. For example, DNA for a signal peptide (secretory leader) is operatively linked to DNA for a polypeptide if it is expressed as a precursor that participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, a B7-H7-binding agent (e.g., an antibody) of the present disclosure is expressed from one or more vectors. In some embodiments, a heavy chain polypeptide is expressed by one vector and a light chain polypeptide is expressed by a second vector. In some embodiments, a heavy chain polypeptide and a light chain polypeptide are expressed by one vector. In some embodiments, a vector encodes a heavy chain polypeptide of a B7-H7 binding agent described herein. In some embodiments, a vector encodes a light chain polypeptide of a B7-H7 binding agent described herein. In some embodiments, a vector encodes a heavy chain polypeptide and a light chain polypeptide of a B7-H7 binding agent described herein.

Suitable host cells for expression of a B7-H7-binding agent (e.g., an antibody) or a B7-H7 protein or fragment thereof to use as an antigen or immunogen include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well-known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present disclosure provides cells comprising the B7-H7-binding agents described herein. In some embodiments, the cells produce the B7-H7-binding agents described herein. In some embodiments, the cells produce an antibody. In some embodiments, the cells produce an antibody that binds human B7-H7. In some embodiments, the cells produce an antibody that binds cyno B7-H7. In some embodiments, the cells produce an antibody that binds human B7-H7 and cyno B7-H7. In some embodiments, the cells produce an antibody designated 2D7. In some embodiments, the cells produce a humanized version of antibody 2D7, referred to as Hz2D7. In some embodiments, the cells produce an antibody designated 2F10. In some embodiments, the cells produce a humanized version of antibody designated 2F10. In some embodiments, the cells produce an antibody designated 3H6. In some embodiments, the cells produce a humanized version of antibody designated 3H6. In some embodiments, the cells produce an antibody designated 5F7. In some embodiments, the cells produce a humanized version of antibody designated 5F7. In some embodiments, the cells produce an antibody designated 16D5. In some embodiments, the cells produce a humanized version of antibody designated 16D5. In some embodiments, the cells produce an antibody designated 28F5. In some embodiments, the cells produce a humanized version of antibody designated 28F5. In some embodiments, the cells produce an antibody designated 1D3. In some embodiments, the cells produce a humanized version of antibody designated 1D3. In some embodiments, the cells produce an antibody designated 5B10. In some embodiments, the cells produce a humanized version of antibody designated 5B10. In some embodiments, the cells produce an antibody designated 5C9. In some embodiments, the cells produce a humanized version of antibody designated 5C9. In some embodiments, the cell is a prokaryotic cell (e.g., E. coli). In some embodiments, the cell is an eukaryotic cell. In some embodiments, the cell is a mammalian cell. In some embodiments, the cell is a hybridoma cell.

Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine (SEQ ID NO: 161), maltose binding domain, influenza coat sequence, and glutathione-S-transferase can be attached to the protein to allow easy purification by passage over an appropriate affinity column. Affinity chromatography used for purifying immunoglobulins include, but are not limited to, Protein A, Protein G, and Protein F chromatography. Isolated proteins can be physically characterized using techniques known to those of skill in the art, including but not limited to, proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems that secrete recombinant protein into culture media are first concentrated using a commercially available protein concentration filter, for example, an Amicon® or Millipore Pellicon® ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In some embodiments, one or more reverse-phase HPLC steps employing hydrophobic RP-HPLC media, e.g., silica gel having pendant methyl or other aliphatic groups, are employed to further purify a recombinant protein. In some embodiments, hydrophobic interaction chromatography (HIC) is used to separate recombinant proteins based on their hydrophobicity. HIC is a useful separation technique for purifying proteins while maintaining biological activity due to the use of conditions and matrices that operate under less denaturing conditions than some other techniques. Some or all of the foregoing purification steps, in various combinations, can be employed to provide a homogeneous recombinant protein.

B7-H7-binding agents (e.g., antibodies) of the present disclosure may be analyzed for their physical/chemical properties and/or biological activities by various methods known in the art. In some embodiments, an anti-B7-H7 antibody is tested for its ability to bind B7-H7 (e.g., human B7-H7 and/or cyno B7-H7). Binding assays include, but are not limited to, SPR (e.g., Biacore), ELISA, and FACS. In some embodiments, an anti-B7-H7 antibody is tested for its ability to inhibit, reduce, or block binding of B7-H7 to its receptor(s). In some embodiments, an anti-B7-H7 antibody is tested for its ability to inhibit, reduce, or block binding of B7-H7 to CD28H. In some embodiments, an anti-B7-H7 antibody is tested for its ability to inhibit, reduce, or block binding of B7-H7 to KIR3DL3. In some embodiments, an anti-B7-H7 antibody is tested for its ability to inhibit, reduce, or block B7-H7 activity. In some embodiments, an anti-B7-H7 antibody is tested for its ability to inhibit, reduce, or block CD28H activity. In some embodiments, an anti-B7-H7 antibody is tested for its ability to inhibit, reduce, or block KIR3DL3 activity. In addition, antibodies may be evaluated for solubility, stability, thermostability, viscosity, expression levels, expression quality, and/or purification efficiency.

In some embodiments, monoclonal antibodies generated against B7-H7 are grouped based upon the epitope each individual antibody recognizes, a process known as “epitope binning”. Generally, antibodies are tested in a pairwise combinatorial manner and antibodies that compete with each other are grouped together into bins. For example, in a premix binning assay, a first antibody is immobilized on a surface and a premixed solution of the second antibody and antigen is flowed over the immobilized first antibody. In parallel, the target protein is immobilized on a surface and the two antibodies are flowed over the immobilized antigen and compete to bind to the target. From this technique, antibodies that block one another can be identified. A competitive blocking profile is created for each antibody relative to the others. The results determine which bin each antibody is placed in. High-throughput methods of epitope binning are known in the art and allow for screening and characterization of large numbers of antibodies. Antibodies that bind similar epitopes often share a similar function. Conversely, antibodies that bind different epitopes may have different functional activities.

In some embodiments, an epitope bin comprises at least one antibody from the group consisting of: 2D7, 2F10, 3H6, 5F7, 16D5, 28F5, 1D3, 5B10, and 5C9. In some embodiments, an epitope bin comprises antibodies 2D7, 2F10, 3H6, 5F7, 16D5, and 28F5. In some embodiments, an epitope bin comprises antibodies 1D3 and 5B10. In some embodiments, an epitope bin comprises antibody 5C9.

Epitope mapping is a method of identifying the binding site, region, or epitope on a target protein where an antibody (or other binding agent) binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g, X-ray crystallography and NMR).

As described herein, the extracellular domain of B7-H7 comprises a sequence of (i) a first IgV-like domain, (ii) an IgC1-like domain, and (iii) a second IgV-like domain. In some studies, these domains are referred to as “domain 1” (first IgV-like domain), “domain 2” (IgC1-like domain), and “domain 3” (second IgV-like domain). In some embodiments, a B7-H7-binding agent (e.g., an antibody) specifically binds within the first IgV-like domain (domain 1). In some embodiments, a B7-H7-binding agent (e.g., an antibody) specifically binds within the IgC1-like domain (domain 2). In some embodiments, a B7-H7-binding agent (e.g., an antibody) specifically binds within the second IgV-like domain (domain 3). In some embodiments, B7-H7-binding agents (e.g., antibodies) are grouped according to the domain of B7-H7 that they bind to (i.e., “domain 1-binding” anti-B7-H7 antibodies).

In some embodiments, B7-H7-binding agents (e.g., antibodies) described herein are characterized by assays including, but not limited to, N-terminal sequencing, amino acid analysis, HPLC, mass spectrometry, ion exchange chromatography, and papain digestion.

In some embodiments, assays are provided for identifying a B7-H7-binding agent (e.g., antibody) that affects B7-H7 activity or a B7-H7 receptor activity. In some embodiments, SPR, ELISA, or FACS assays are used to assess the ability of a B7-H7-binding agent to block binding of B7-H7 to CD28H and/or KIR3DL3. In some embodiments, cytotoxicity assays are used to assess the ability of a B7-H7-binding agent to affect natural killer (NK) cell activity. In some embodiments, proliferation assays are used to assess the ability of a B7-H7-binding agent to affect T-cell activity.

In some embodiments, a B7-H7-binding agent (e.g., an antibody) described herein is an antagonist of human B7-H7. In some embodiments, a B7-H7-binding agent induces, enhances, and/or increases an immune response. In some embodiments, a B7-H7-binding agent activates and/or increases activity of NK cells and/or T-cells (e.g., cytolytic activity or cytokine production). In some embodiments, a B7-H7-binding agent inhibits B7-H7 suppression of NK cell activity. In some embodiments, the terms “inhibiting”, “reducing”, “inducing”, “enhancing”, and “increasing” are relative to levels and/or activity in the absence of treatment with the B7-H7-binding agent. In some embodiments, the terms “inhibiting”, “reducing”, “inducing”, “enhancing”, and “increasing” are relative to levels and/or activity prior to treatment with the B7-H7-binding agent.

In certain embodiments, a B7-H7-binding agent (e.g., an antibody) described herein increases cytolytic activity of a NK cell. In certain embodiments, a B7-H7-binding agent increases the activity of NK cells at least about 10%, at least about 25%, at least about 50%, at least about 75%, at least about 90%, or at least about 95%.

In certain embodiments, a B7-H7-binding agent (e.g., an antibody) described herein increases activation and/or proliferation of a T-cell. In certain embodiments, a B7-H7-binding agent increases the activation and/or proliferation of a T-cell at least about 10%, at least about 25%, at least about 50%, at least about 75%, at least about 90%, or at least about 95%.

The present disclosure also provides conjugates comprising a B7-H7-binding agent (e.g., an antibody) described herein. In some embodiments, an anti-B7-H7 antibody is attached to a second molecule. In some embodiments, an anti-B7-H7 antibody is conjugated to a cytotoxic agent or moiety. In some embodiments, an anti-B7-H7 antibody is conjugated to a cytotoxic agent to form an ADC (antibody-drug conjugate). In some embodiments, the cytotoxic moiety is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DM1 and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleuritesfordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody is conjugated to one or more small molecule toxins, such as calicheamicins, maytansinoids, trichothenes, and CC1065. A derivative of any one of these toxins may be used as long as the derivative retains the cytotoxic activity of the parent molecule.

Conjugates comprising a B7-H7-binding agent (e.g., an antibody) described herein may be made using any suitable method known in the art. In some embodiments, conjugates are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, a B7-H7-binding agent (e.g., an antibody) described herein is conjugated to a detectable substance or molecule that allows the agent to be used for diagnosis and/or detection. A detectable substance can include, but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷SC, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions.

An anti-B7-H7 antibody described herein can also be conjugated to a second antibody to form an antibody heteroconjugate.

A B7-H7-binding agent (e.g., an antibody) described herein may be attached to a solid support. Such solid supports include, but are not limited to, glass, cellulose, polyacrylamide, nylon, polystyrene, polyvinyl chloride, or polypropylene. In some embodiments, an immobilized anti-B7-H7 antibody is used in an immunoassay. In some embodiments, an immobilized anti-B7-H7 antibody is used in purification of the target antigen (e.g., human B7-H7 or cyno B7-H7).

### III. Polynucleotides

In some embodiments, the disclosure encompasses polynucleotides comprising polynucleotides that encode a polypeptide (e.g., a B7-H7-binding agent) described herein. The term “polynucleotides that encode a polypeptide” encompasses a polynucleotide that includes only coding sequences for the polypeptide as well as a polynucleotide that includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, a polynucleotide comprises a polynucleotide (e.g., a nucleotide sequence) encoding a heavy chain of a B7-H7-binding agent (e.g., antibody) described herein. In some embodiments, a polynucleotide comprises a polynucleotide encoding a light chain of a B7-H7-binding agent (e.g., antibody) described herein. In some embodiments, a polynucleotide comprises a polynucleotide encoding a heavy chain of a B7-H7-binding agent (e.g, antibody) described herein and a polynucleotide encoding a light chain of the B7-H7-binding agent (e.g., antibody).

In some embodiments, the polynucleotide comprises a polynucleotide (e.g., a nucleotide sequence) encoding a polypeptide comprising an amino acid sequence selected from the group consisting of: SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:32, SEQ ID NO:33, SEQ ID NO:36, SEQ ID NO:37, SEQ ID NO:41, SEQ ID NO:42, SEQ ID NO:47, SEQ ID NO:48, SEQ ID NO:55, SEQ ID NO:56, SEQ ID NO:63, SEQ ID NO:64, SEQ ID NO:67, SEQ ID NO:68, SEQ ID NO:75, and SEQ ID NO:76. In some embodiments, the polynucleotide comprises a polynucleotide (e.g., a nucleotide sequence) encoding a polypeptide comprising an amino acid sequence of SEQ ID NO: 19. In some embodiments, the polynucleotide comprises a polynucleotide encoding a polypeptide comprising an amino acid sequence of SEQ ID NO:20. In some embodiments, the polynucleotide comprises a polynucleotide encoding a polypeptide comprising an amino acid sequence of SEQ ID NO:21. In some embodiments, the polynucleotide comprises a polynucleotide encoding a polypeptide comprising an amino acid sequence of SEQ ID NO:22. In some embodiments, the polynucleotide comprises a polynucleotide encoding a polypeptide comprising an amino acid sequence of SEQ ID NO:23. In some embodiments, the polynucleotide comprises a polynucleotide encoding a polypeptide comprising an amino acid sequence of SEQ ID NO:24. In some embodiments, the polynucleotide comprises a polynucleotide encoding a polypeptide comprising an amino acid sequence of SEQ ID NO:25. In some embodiments, the polynucleotide comprises a polynucleotide encoding a polypeptide comprising an amino acid sequence of SEQ ID NO:26. In some embodiments, the polynucleotide comprises a polynucleotide encoding a polypeptide comprising an amino acid sequence of SEQ ID NO:32. In some embodiments, the polynucleotide comprises a polynucleotide encoding a polypeptide comprising an amino acid sequence of SEQ ID NO:33. In some embodiments, the polynucleotide comprises a polynucleotide encoding a polypeptide comprising an amino acid sequence of SEQ ID NO:36. In some embodiments, the polynucleotide comprises a polynucleotide encoding a polypeptide comprising an amino acid sequence of SEQ ID NO:37. In some embodiments, the polynucleotide comprises a polynucleotide encoding a polypeptide comprising an amino acid sequence of SEQ ID NO:41. In some embodiments, the polynucleotide comprises a polynucleotide encoding a polypeptide comprising an amino acid sequence of SEQ ID NO:42. In some embodiments, the polynucleotide comprises a polynucleotide encoding a polypeptide comprising an amino acid sequence of SEQ ID NO:47. In some embodiments, the polynucleotide comprises a polynucleotide encoding a polypeptide comprising an amino acid sequence of SEQ ID NO:48. In some embodiments, the polynucleotide comprises a polynucleotide encoding a polypeptide comprising an amino acid sequence of SEQ ID NO:55. In some embodiments, the polynucleotide comprises a polynucleotide encoding a polypeptide comprising an amino acid sequence of SEQ ID NO:56. In some embodiments, the polynucleotide comprises a polynucleotide encoding a polypeptide comprising an amino acid sequence of SEQ ID NO:63. In some embodiments, the polynucleotide comprises a polynucleotide encoding a polypeptide comprising an amino acid sequence of SEQ ID NO:64. In some embodiments, the polynucleotide comprises a polynucleotide encoding a polypeptide comprising an amino acid sequence of SEQ ID NO:67. In some embodiments, the polynucleotide comprises a polynucleotide encoding a polypeptide comprising an amino acid sequence of SEQ ID NO:68. In some embodiments, the polynucleotide comprises a polynucleotide encoding a polypeptide comprising an amino acid sequence of SEQ ID NO:75. In some embodiments, the polynucleotide comprises a polynucleotide encoding a polypeptide comprising an amino acid sequence of SEQ ID NO:76.

In some embodiments, the polynucleotide comprises a polynucleotide (e.g., a nucleotide sequence) encoding a polypeptide comprising more than one amino acid sequence selected from the group consisting of: SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:32, SEQ ID NO:33, SEQ ID NO:36, SEQ ID NO:37, SEQ ID NO:41, SEQ ID NO:42, SEQ ID NO:47, SEQ ID NO:48, SEQ ID NO:55, SEQ ID NO:56, SEQ ID NO:63, SEQ ID NO:64, SEQ ID NO:67, SEQ ID NO:68, SEQ ID NO:75, and SEQ ID NO:76. In some embodiments, the polynucleotide comprises a polynucleotide encoding (i) a polypeptide comprising an amino acid sequence of SEQ ID NO: 19 and (ii) a polypeptide comprising an amino acid sequence of SEQ ID NO:20. In some embodiments, the polynucleotide comprises a polynucleotide encoding (i) a polypeptide comprising an amino acid sequence of SEQ ID NO:21 and (ii) a polypeptide comprising an amino acid sequence of SEQ ID NO:22. In some embodiments, the polynucleotide comprises a polynucleotide encoding (i) a polypeptide comprising an amino acid sequence of SEQ ID NO:23 and (ii) a polypeptide comprising an amino acid sequence of SEQ ID NO:25. In some embodiments, the polynucleotide comprises a polynucleotide encoding (i) a polypeptide comprising an amino acid sequence of SEQ ID NO:24 and (ii) a polypeptide comprising an amino acid sequence of SEQ ID NO:26. In some embodiments, the polynucleotide comprises a polynucleotide encoding (i) a polypeptide comprising an amino acid sequence of SEQ ID NO:32 and (ii) a polypeptide comprising an amino acid sequence of SEQ ID NO:33. In some embodiments, the polynucleotide comprises a polynucleotide encoding (i) a polypeptide comprising an amino acid sequence of SEQ ID NO:36 and (ii) a polypeptide comprising an amino acid sequence of SEQ ID NO:37. In some embodiments, the polynucleotide comprises a polynucleotide encoding (i) a polypeptide comprising an amino acid sequence of SEQ ID NO:41 and (ii) a polypeptide comprising an amino acid sequence of SEQ ID NO:42. In some embodiments, the polynucleotide comprises a polynucleotide encoding (i) a polypeptide comprising an amino acid sequence of SEQ ID NO:47 and (ii) a polypeptide comprising an amino acid sequence of SEQ ID NO:48. In some embodiments, the polynucleotide comprises a polynucleotide encoding (i) a polypeptide comprising an amino acid sequence of SEQ ID NO:55 and (ii) a polypeptide comprising an amino acid sequence of SEQ ID NO:56. In some embodiments, the polynucleotide comprises a polynucleotide encoding (i) a polypeptide comprising an amino acid sequence of SEQ ID NO:63 and (ii) a polypeptide comprising an amino acid sequence of SEQ ID NO:64. In some embodiments, the polynucleotide comprises a polynucleotide encoding (i) a polypeptide comprising an amino acid sequence of SEQ ID NO:67 and (ii) a polypeptide comprising an amino acid sequence of SEQ ID NO:68. In some embodiments, the polynucleotide comprises a polynucleotide encoding (i) a polypeptide comprising an amino acid sequence of SEQ ID NO:75 and (ii) a polypeptide comprising an amino acid sequence of SEQ ID NO:76.

The present disclosure also provides variants of the polynucleotides described herein, wherein the variant encodes, for example, fragments, analogs, and/or derivatives of a polypeptide. In some embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide described herein.

In some embodiments, a polynucleotide comprises a polynucleotide having a nucleotide sequence at least about 80% identical, at least about 85% identical, at least about 90% identical, at least about 95% identical, and in some embodiments, at least about 96%, 97%, 98%, or 99% identical to a polynucleotide encoding an amino acid sequence selected from the group consisting of: SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:32, SEQ ID NO:33, SEQ ID NO:36, SEQ ID NO:37, SEQ ID NO:41, SEQ ID NO:42, SEQ ID NO:47, SEQ ID NO:48, SEQ ID NO:55, SEQ ID NO:56, SEQ ID NO:63, SEQ ID NO:64, SEQ ID NO:67, SEQ ID NO:68, SEQ ID NO:75, and SEQ ID NO:76. Also provided is a polynucleotide that comprises a polynucleotide that hybridizes to a polynucleotide encoding an amino acid sequence selected from the group consisting of: SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:32, SEQ ID NO:33, SEQ ID NO:36, SEQ ID NO:37, SEQ ID NO:41, SEQ ID NO:42, SEQ ID NO:47, SEQ ID NO:48, SEQ ID NO:55, SEQ ID NO:56, SEQ ID NO:63, SEQ ID NO:64, SEQ ID NO:67, SEQ ID NO:68, SEQ ID NO:75, and SEQ ID NO:76. In some embodiments, the hybridization is under conditions of high stringency as is known to those skilled in the art.

As used herein, the phrase “a polynucleotide having a nucleotide sequence at least, for example, 95% identical to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations that produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). In some embodiments, a polynucleotide variant comprises one or more mutated codons comprising one or more (e.g., 1, 2, or 3) substitutions to the codon that change the amino acid encoded by that codon. Methods for introducing one or more substitutions into a codon are known in the art, including but not limited to, PCR mutagenesis and site-directed mutagenesis. Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide (e.g., an antibody) fused in the same reading frame to a polynucleotide that aids in expression and secretion of a polypeptide from a host cell (e.g., a leader sequence that functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine (SEQ ID NO: 161) tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker is used in conjunction with other markers or tags.

In some embodiments, the polynucleotides are isolated. In some embodiments, the polynucleotides are substantially pure.

Vectors and cells comprising the polynucleotides described herein are also provided. In some embodiments, an expression vector comprises a polynucleotide molecule encoding a B7-H7-binding agent (e.g., an antibody) described herein. In some embodiments, an expression vector comprises a polynucleotide molecule encoding a polypeptide that is part of a B7-H7-binding agent described herein. In some embodiments, a host cell comprises an expression vector comprising the polynucleotide molecule encoding a B7-H7-binding agent described herein. In some embodiments, a host cell comprises an expression vector comprising the polynucleotide molecule encoding a polypeptide that is part of a B7-H7-binding agent described herein. In some embodiments, a host cell comprises a polynucleotide molecule encoding a B7-H7-binding agent described herein. In some embodiments, a cell comprises one or more polynucleotides encoding a B7-H7-binding agent described herein. In some embodiments, a cell comprises a single polynucleotide encoding a B7-H7-binding agent described herein. In some embodiments, a cell comprises a first polynucleotide encoding a heavy chain of a B7-H7-binding agent described herein and a second polynucleotide encoding a light chain of a B7-H7-binding agent described herein. In some embodiments, a cell comprises a polynucleotide encoding a heavy chain and a light chain of a B7-H7-binding agent described herein. In some embodiments, a cell comprises one or more vectors encoding a B7-H7 binding agent described herein. In some embodiments, a cell comprises a vector encoding a B7-H7-binding agent described herein. In some embodiments, a cell comprises a first vector encoding a heavy chain of a B7-H7-binding agent described herein and a second vector encoding a light chain of a B7-H7-binding agent described herein. In some embodiments, a cell comprises a single vector encoding a heavy chain and a light chain of a B7-H7-binding agent described herein.

### IV. Methods of Making Binding Agents

The disclosure provides methods for making the B7-H7-binding agents (e.g., antibodies) described herein. In some embodiments, a method comprises providing a cell comprising a heavy chain and/or light chain of a B7-H7-binding agent (e.g., an antibody), incubating the cell under conditions that permit the expression of the binding agent, and isolating the binding agent. In certain embodiments, the cell comprises one or more vectors encoding the heavy chain and the light chain of an anti-B7-H7 antibody described herein. In some embodiments, a cell comprises a first vector encoding the heavy chain of an anti-B7-H7 antibody described herein and a second vector encoding the light chain an anti-B7-H7 antibody described herein. In other embodiments, a cell comprises a vector encoding the heavy chain and the light chain of an anti-B7-H7 antibody described herein. In certain embodiments, a ceil comprises one or more polynucleotides encoding the heavy chain and the light chain of an anti-B7-H7 antibody described herein. In some embodiments, a cell comprises a first polynucleotide encoding the heavy chain of an anti-B7-H7 antibody described herein and a second poly nucleotide encoding the light chain of an anti-B7-H7 antibody described herein. In other embodiments, a cell comprises a polynucleotide encoding the heavy chain and the light chain of an anti-B7-H7 antibody described herein. In some embodiments, the method comprises purifying the antibody. In certain embodiments, the cell is a CHQ cell. In some embodiments, the cell is a 293 cell. In certain embodiments, the cell is a bacterial cell (e.g., E. coli).

### V. Methods of Use and Pharmaceutical Compositions

The B7-H7-binding agents (e.g., antibodies) of the disclosure are useful in a variety of applications including, but not limited to, therapeutic treatment methods. In some embodiments, a B7-H7-binding agent described herein is useful in methods for modulating an immune response. In some embodiments, a B7-H7-binding agent described herein is useful in methods for inducing, activating, promoting, increasing, enhancing, or prolonging an immune response. In some embodiments, a B7-H7-binding agent described herein is useful in methods for increasing an immune response. In some embodiments, a B7-H7-binding agent described herein is useful in methods for treating cancer. In some embodiments, a B7-H7-binding agent described herein is useful in methods for inhibiting tumor growth. In any of the methods described herein, the subject may be a human subject.

In some embodiments, a method of modulating an immune response in a subject comprises administering to the subject a therapeutically effective amount of a B7-H7-binding agent described herein. In some embodiments, a method of modulating an immune response in a subject comprises administering to the subject a therapeutically effective amount of an anti-B7-H7 antibody described herein. In some embodiments, a method of inducing, activating, promoting, increasing, enhancing, or prolonging an immune response in a subject comprises administering to the subject a therapeutically effective amount of a B7-H7-binding agent described herein. In some embodiments, a method of inducing, activating, promoting, increasing, enhancing, or prolonging an immune response in a subject comprises administering to the subject a therapeutically effective amount of an anti-B7-H7 antibody described herein. In some embodiments, the inducing, activating, promoting, increasing, enhancing, or prolonging of an immune response comprises increasing NK cell activity. In some embodiments, the immune response is against a tumor. In some embodiments, the immune response is against cancer. In some embodiments, the immune response is in response to an antigenic stimulation. In some embodiments, the antigenic stimulation is a tumor cell. In some embodiments, the antigenic stimulation is a cancer cell or cancer.

In some embodiments, a method of increasing NK cell activity in a subject comprises administering to the subject a therapeutically effective amount of a B7-H7-binding agent described herein. In some embodiments, a method of increasing NK cell activity in a subject comprises administering to the subject a therapeutically effective amount of an anti-B7-H7 antibody described herein.

In some embodiments, a method of inhibiting suppression of NK cell activity in a subject comprises administering to the subject a therapeutically effective amount of a B7-H7-binding agent described herein. In some embodiments, a method of inhibiting suppression of NK cell activity in a subject comprises administering to the subject a therapeutically effective amount of an anti-B7-H7 antibody described herein.

In some embodiments, a method of inhibiting growth of tumor cells comprises contacting the tumor cells with an effective amount of a B7-H7-binding agent described herein. In some embodiments, a method of inhibiting growth of tumor cells comprises contacting the tumor cells with an effective amount of an anti-B7-H7 antibody described herein. In certain embodiments, the method of inhibiting growth of a tumor comprises contacting a cell mixture with a B7-H7-binding agent (e.g., antibody) in vitro. For example, an immortalized cell line or a cancer cell line mixed with immune cells (e.g, T-cells and/or NK cells) is cultured in medium to which is added a test B7-H7-binding agent. In some embodiments, tumor cells are isolated from a patient sample such as, for example, a tissue biopsy, pleural effusion, or blood sample, mixed with immune cells (e.g., T-cells and/or NK cells), and cultured in medium to which is added a test B7-H7-binding agent. In some embodiments, the invention provides use of a B7-H7-binding agent (e.g., antibody) described herein in the manufacture or preparation of a medicament for inhibiting growth of a tumor or a tumor cell. In some embodiments, a B7-H7-binding agent increases, promotes, and/or enhances the activity of the immune cells. In some embodiments, a B7-H7-binding agent inhibits tumor cell growth.

In some embodiments, the method of inhibiting growth of a tumor comprises contacting the tumor or tumor cells with a B7-H7-binding agent (e.g., antibody) described herein in vivo. In certain embodiments, contacting a tumor or tumor cell with a B7-H7-binding agent is undertaken in an animal model. For example, a test B7-H7-binding agent may be administered to mice that have tumors. In some embodiments, a B7-H7-binding agent increases, promotes, and/or enhances the activity of immune cells in the mice. In some embodiments, a B7-H7-binding agent inhibits tumor growth. In some embodiments, a B7-H7-binding agent causes a tumor to regress. In some embodiments, a B7-H7-binding agent is administered at the same time or shortly after introduction of tumor cells into the animal to prevent tumor growth (“preventative model”). In some embodiments, a B7-H7-binding agent is administered as a therapeutic after tumors have grown to a specified size or have become “established” (“therapeutic model”).

In some embodiments, a method of inhibiting growth of a tumor in a subject comprises administering to the subject a therapeutically effective amount of a B7-H7-binding agent described herein. In some embodiments, a method of inhibiting growth of a tumor in a subject comprises administering to the subject a therapeutically effective amount of an anti-B7-H7 antibody described herein. In some embodiments, the tumor expresses B7-H7. In certain embodiments, the subject is a human. In certain embodiments, the subject has a tumor or the subject had a tumor that was at least partially removed.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, bone tumor, glioblastoma, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In certain embodiments, the tumor is a melanoma tumor. In certain embodiments, the tumor is a breast tumor. In certain embodiments, the tumor is a liver tumor. In certain embodiments, the tumor is a prostate tumor. In certain embodiments, the tumor is a kidney tumor.

In some embodiments, a method of treating cancer in a subject comprises administering to the subject a therapeutically effective amount of a B7-H7-binding agent described herein. In some embodiments, a method of treating cancer in a subject comprises administering to the subject a therapeutically effective amount of an anti-B7-H7 antibody described herein. In some embodiments, the cancer expresses B7-H7.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, bone cancer, glioblastoma, and head and neck cancer. In certain embodiments, the cancer is colorectal cancer. In certain embodiments, the cancer is ovarian cancer. In certain embodiments, the cancer is lung cancer. In certain embodiments, the cancer is pancreatic cancer. In certain embodiments, the cancer is melanoma. In certain embodiments, the cancer is breast cancer. In certain embodiments, the cancer is liver cancer. In certain embodiments, the cancer is prostate cancer. In certain embodiments, the cancer is kidney cancer.

In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody 2D7. In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody Hz2D7. In some embodiments of the methods described herein, a B7-H7-binding agent comprises a heavy chain variable region CDR1, CDR2, and CDR3 and a light chain variable region CDR1, CDR2, and CDR3 of Table 1. In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody 2F10. In some embodiments of the methods described herein, a B7-H7-binding agent comprises a heavy chain variable region CDR1, CDR2, and CDR3 and a light chain variable region CDR1, CDR2, and CDR3 of Table 2. In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody 3H6. In some embodiments of the methods described herein, a B7-H7-binding agent comprises a heavy chain variable region CDR1, CDR2, and CDR3 and a light chain variable region CDR1, CDR2, and CDR3 of Table 3. In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody 5F7. In some embodiments of the methods described herein, a B7-H7-binding agent comprises a heavy chain variable region CDR1, CDR2, and CDR3 and a light chain variable region CDR1, CDR2, and CDR3 of Table 4. In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody 16D5. In some embodiments of the methods described herein, a B7-H7-binding agent comprises a heavy chain variable region CDR1, CDR2, and CDR3 and a light chain variable region CDR1, CDR2, and CDR3 of Table 5. In some embodiments of the methods described herein, the C3-binding agent (e.g. anti-C3 antibody) comprises a heavy chain variable region CDR1, CDR2, and CDR3 and a light chain variable region CDR1, CDR2, and CDR3 of antibody 28F5. In some embodiments of the methods described herein, a B7-H7-binding agent comprises a heavy chain variable region CDR1, CDR2, and CDR3 and a light chain variable region CDR1, CDR2, and CDR3 of Table 6. In some embodiments of the methods described herein, a B7-H7-binding agent (e.g, an anti-B7-H7 antibody) comprises a heavy chain variable region CDR1, CDR2, and CDR3 and a light chain variable region CDR1, CDR2, and CDR3 of antibody 1D3. In some embodiments of the methods described herein, a B7-H7-binding agent comprises a heavy chain variable region CDR1, CDR2, and CDR3 and a light chain variable region CDR1, CDR2, and CDR3 of Table 7. In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody 5B10. In some embodiments of the methods described herein, a B7-H7-binding agent comprises a heavy chain variable region CDR1, CDR2, and CDR3 and a light chain variable region CDR1, CDR2, and CDR3 of Table 8. In some embodiments of the methods described herein, a B7-H7-binding agent (e.g., an anti-B7-H7 antibody) comprises a heavy chain variable region CDR1, CDR2, and CDR3 and a light chain variable region CDR1, CDR2, and CDR3 of antibody 5C9. In some embodiments of the methods described herein, a B7-H7-binding agent comprises a heavy chain variable region CDR1, CDR2, and CDR3 and a light chain variable region CDR1, CDR2, and CDR3 of Table 9.

In some embodiments of the methods described herein, the B7-H7-binding agent is a humanized anti-B7-H7 antibody having the above specified CDRs.

In some embodiments of the methods described herein, the anti-B7-H7 antibody comprises a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15), and a light chain variable region CDR1 comprising KASQDVGTAVA (SEQ ID NO: 16); a light chain variable region CDR2 comprising WAFTRHT (SEQ ID NO: 17); and a light chain variable region CDR3 comprising QQHYDTPFT (SEQ ID NO: 18.

In some embodiments of the methods described herein, the anti-B7-H7 antibody comprises: (a) a heavy chain variable region of SEQ ID NO: 19 or SEQ ID NO:21; and (b) a light chain variable region of SEQ ID NO:20 or SEQ ID NO:22. In some embodiments of the methods described herein, the anti-B7-H7 antibody comprises a heavy chain variable region of SEQ ID NO: 19 and a light chain variable region of SEQ ID NO:20. In some embodiments of the methods described herein, the anti-B7-H7 antibody comprises a heavy chain variable region of SEQ ID NO:21 and a light chain variable region of SEQ ID NO:22. In some embodiments of the methods described herein, the anti-B7-H7 antibody is 2D7. In some embodiments of the methods described herein, the anti-B7-H7 antibody is Hz2D7.

In some embodiments of the methods described herein, the anti-B7-H7 antibody comprises a polypeptide of SEQ ID NO:24 and a polypeptide of SEQ ID NO:26.

In some embodiments of the methods described herein, a method comprises administering a B7-H7-binding agent described herein in combination with at least one additional therapeutic agent or therapeutic therapy. In some embodiments, the at least one additional therapeutic agent comprises 1, 2, 3, or more additional therapeutic agents. Treatment with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistance to an agent will develop.

In some embodiments, the combination of a B7-H7-binding agent described herein and at least one additional therapeutic agent results in additive or synergistic results. In some embodiments, the combination therapy results in an increase in the therapeutic index of the B7-H7-binding agent. In some embodiments, the combination therapy results in an increase in the therapeutic index of the additional therapeutic agent(s). In some embodiments, the combination therapy results in a decrease in the toxicity and/or side effects of the B7-H7-binding agent. In some embodiments, the combination therapy results in a decrease in the toxicity and/or side effects of the additional therapeutic agent(s).

Therapeutic agents that may be administered in combination with the B7-H7-binding agents described herein include chemotherapeutic agents. Thus, in some embodiments, the method or treatment involves the administration of a B7-H7-binding agent of the present invention in combination with a chemotherapeutic agent or in combination with a cocktail of chemotherapeutic agents.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in combination therapy include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K (PSK); razoxane; sizofirran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); toxoids such as paclitaxel (TAXOU) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; irinotecan (CPT-11); topoisomerase inhibitor RFS 2000; difluoromethylomithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plkl. In certain embodiments, the additional therapeutic agent is paclitaxel. In certain embodiments, the additional therapeutic agent is albumin-bound paclitaxel (ABRAXANE).

In some embodiments, an additional therapeutic agent comprises an agent such as a small molecule. For example, treatment can involve the combined administration of an agent of the present invention with a small molecule that acts as an inhibitor against tumor-associated antigens including, but not limited to, EGFR, HER2 (ErbB2), and/or VEGF. In some embodiments, an agent of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In some embodiments, in addition to administering a B7-H7-binding agent described herein, a method or treatment further comprises administering at least one immunotherapeutic agent. In some embodiments, an immunotherapeutic agent is an immunomodulatory agent. In some embodiments, an immunotherapeutic agent is an immune response stimulating agent. In some embodiments, the immunotherapeutic agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g, IL-1, IL2, IL-3, IL-7, IL-12, IL-15, IL-18), an antibody that blocks immunosuppressive functions (e.g., an anti-CTLA4 antibody, anti-CD28 antibody, anti-CD3 antibody, anti-PD-1 antibody, anti-PD-L1 antibody), an antibody that enhances immune cell functions (e.g., an anti-GITR antibody or an anti-OX-40 antibody), a toll-like receptor (e.g., TLR4, TLR7, TLR9), a soluble ligand (e.g., GITRL or OX-40L), or a member of the B7 family (e.g, CD80, CD86). In some embodiments, the immunotherapeutic agent is an anti-PD-1 antibody. In some embodiments, the immunotherapeutic agent is an anti-PD-L1 antibody.

In some embodiments, the additional therapeutic agent is an immune checkpoint inhibitor. In some embodiments, the immune checkpoint inhibitor is an anti-PD-1 antibody, an anti-PD-L1 antibody, an anti-CTLA4 antibody, an anti-CD28 antibody, an anti-LAG3 antibody, an anti-TIM3 antibody, an anti-GITR antibody, an anti-OX-40 antibody, or an anti-4-1BB antibody. In some embodiments, the additional therapeutic agent is an anti-PD-1 antibody selected from the group consisting of: nivolumab (OPDIVO), pembrolizumab (KEYTRUDA), or pidilzumab. In some embodiments, the additional therapeutic agent is an anti-PD-1 antibody selected from the group consisting of: MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MEDI4736), or avelumab (MSB0010718C). In some embodiments, the additional therapeutic agent is an anti-CTLA-4 antibody selected from the group consisting of: ipilimumab (YERVOY) or tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

It will be appreciated that the combination of a B7-H7-binding agent described herein and at least one additional therapeutic agent can be administered in any order or concurrently. In some embodiments, an additional therapeutic agent is administered prior to administration of the B7-H7-binding agent. In some embodiments, an additional therapeutic agent is administered concurrently with administration of the B7-H7-binding agent. In some embodiments, an additional therapeutic agent is administered subsequently to administration of the B7-H7-binding agent. Administration can include co-administration, either in a single pharmaceutical formulation or using separate formulations, or consecutive administration in either order but generally within a time period such that the active agents can exert their biological activities.

In some embodiments, the B7-H7-binding agent is administered to subjects that have previously undergone treatment with a therapeutic agent. In some embodiments, the B7-H7-binding agent and a second therapeutic agent is administered substantially simultaneously or concurrently. For example, a subject may be given a B7-H7-binding agent while undergoing a course of treatment with a second therapeutic agent (e.g., chemotherapeutic agent). In some embodiments, a B7-H7-binding agent is administered within 1 year of the treatment with a second therapeutic agent. In some embodiments, a B7-H7-binding agent is administered within 10, 8, 6, 4, or 2 months of any treatment with a second therapeutic agent. In some embodiments, a B7-H7-binding agent is administered within 4, 3, 2, or 1 weeks of any treatment with a second therapeutic agent. In some embodiments, a B7-H7-binding agent is administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

Preparation and dosing schedules for additional therapeutic agents can be used according to manufacturers' instructions or as determined empirically by the skilled practitioner. For example, preparation and dosing schedules for chemotherapy are described in The Chemotherapy Source Book 4th Edition, 2008, M. C. Perry, Editor, Fippincott, Williams & Wilkins, Philadelphia, Pa.

The dose of a B7-H7-binding agent described herein may vary depending on the nature and/or severity of the disease or disorder, as well as the condition of the subject. In some embodiments, dosage of the agent is from 0.01 μg/kg to 100 mg/kg of body weight, from 0.1 μg/kg to 100 mg/kg of body weight, from 1 μg/kg to 100 mg/kg of body weight, from 1 mg/kg to 100 mg/kg of body weight, 1 mg/kg to 80 mg/kg of body weight, from 1 mg/kg to 50 mg/kg of body weight, from 1 mg/kg to 25 mg/kg of body weight, from 1 mg/kg to 15 mg/kg of body weight, from 10 mg/kg to 100 mg/kg of body weight, from 10 mg/kg to 75 mg/kg of body weight, or from 10 mg/kg to 50 mg/kg of body weight. In some embodiments, dosage of the agent is from about 0.1 mg to about 20 mg/kg of body weight. In some embodiments, dosage of the agent is about 0.5 mg/kg of body weight. In some embodiments, dosage of the agent is about 1 mg/kg of body weight. In some embodiments, dosage of the agent is about 1.5 mg/kg of body weight. In some embodiments, dosage of the agent is about 2 mg/kg of body weight. In some embodiments, dosage of the agent is about 2.5 mg/kg of body weight. In some embodiments, dosage of the agent is about 5 mg/kg of body weight. In some embodiments, dosage of the agent is about 7.5 mg/kg of body weight. In some embodiments, dosage of the agent is about 10 mg/kg of body weight. In some embodiments, dosage of the agent is about 12.5 mg/kg of body weight. In some embodiments, dosage of the agent is about 15 mg/kg of body weight. In some embodiments, the agent is dosed once or more daily, weekly, monthly, or yearly. In some embodiments, the agent is dosed once every week, once every two weeks, once every three weeks, or once every four weeks. In some embodiments, the agent is dosed once a week. In some embodiments, the agent is dosed once every two weeks. In some embodiments, the agent is dosed once every three weeks. In some embodiments, the agent is dosed once every four weeks.

The present disclosure provides compositions comprising a B7-H7-binding agent described herein. The present disclosure also provides pharmaceutical compositions comprising a B7-H7-binding agent described herein and a pharmaceutically acceptable vehicle.

In some embodiments, the pharmaceutical composition comprising a B7-H7-binding further comprises an anti-tumor agent. In some embodiments, the anti-tumor agent is a checkpoint inhibitor. In some embodiments, the checkpoint inhibitor is selected from the group consisting of an anti-PD-1 antibody, anti-PD-L1 antibody, an anti-CTLA4 antibody, an anti-LAG3 antibody, an anti-TIM3 antibody, an anti-GITR antibody, an anti-OX40 antibody, an anti-4-1-BB antibody and an anti-CD28 antibody.

Formulations are prepared for storage and/or use by combining a B7-H7-binding agent (e.g., antibody) of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{nd} Edition, 2012, Pharmaceutical Press, London). In some embodiments, the formulation is in the form of an aqueous solution. In some embodiments, the formulation is lyophilized or in an alternative dried form.

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol, and cellulose acetate.

The binding agents of the present disclosure may be formulated in any suitable form for delivery to a target cell/tissue. In some embodiments, a B7-H7-binding agent can be formulated as a liposome, microparticle, microcapsule, albumin microsphere, microemulsion, nano-particle, nanocapsule, or macroemulsion. In some embodiments, the pharmaceutical formulation includes a B7-H7-binding agent of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE).

In some embodiments, a B7-H7-binding agent is formulated as a sustained-release preparation. Suitable examples of sustained-release preparations include semi-permeable matrices of solid hydrophobic polymers containing an agent, where the matrices are in the form of shaped articles (e.g., films or microcapsules). Sustained-release matrices include but are not limited to polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions or formulations of the present disclosure can be administered in any number of ways for either local or systemic treatment. In some embodiments, administration is topical by epidermal or transdermal patches, ointments, lotions, creams, gels, drops, suppositories, sprays, liquids and powders. In some embodiments, administration is pulmonary by inhalation or insufflation of powders or aerosols, including by nebulizer, intratracheal, and intranasal. In some embodiments, administration is oral. In some embodiments, administration is parenteral including intravenous, intraarterial, intratumoral, subcutaneous, intraperitoneal, intramuscular (e.g., injection or infusion), or intracranial (e.g., intrathecal or intraventricular). In some embodiments, administration is by intravenous injection or intravenous infusion.

Various delivery systems are known and can be used to administer a B7-H7-binding agent described herein. In some embodiments, a B7-H7-binding agent or a composition described herein is delivered in a controlled release or sustained release system. In some embodiments, a pump is used to achieve controlled or sustained release. In some embodiments, polymeric materials are used to achieve controlled or sustained release of the B7-H7-binding agent described herein. Examples of polymers used in sustained release formulations include, but are not limited to, poly 2-hydroxy ethyl methacrylate, polymethyl methacrylate, polyacrylic acid, polyethylene-co-vinyl acetate, polymethacrylic acid, polyglycolides (PLG), polyanhydrides, poly N-vinyl pyrrolidone, polyvinyl alcohol (PVA), polyacrylamide, polyethylene glycol (PEG), polylactides (PLA), polylactide-co-glycolides (PLGA), and polyorthoesters. Any polymer used in a sustained release formulation should be inert, free of leachable impurities, stable on storage, sterile, and biodegradable.

Additional delivery systems can be used to administer a B7-H7-binding agent described herein including, but not limited to, injectable drug delivery devices and osmotic pumps. Injectable drug delivery devices include, for example, hand-held devices (e.g., autoinjectors) or wearable devices. Different types of osmotic pump systems may include single compartment systems, dual compartment systems, and multiple compartment systems.

### VI. Assays and/or Kits Comprising B7-H7-Binding Agents

In some embodiments, the B7-H7-binding agents (e.g., anti-B7-H7 antibodies) described herein are useful for detecting the presence of B7-H7 in a biological sample. In some embodiments, a B7-H7 binding agent is an anti-B7-H7 antibody that binds human and/or cyno B7-H7, but does not inhibit B7-H7 activity. The term “detecting” as used herein encompasses quantitative or qualitative detection. In some embodiments, a biological sample comprises a cell, tissue, blood, or other bodily fluid.

In some embodiments, a method of detecting the presence of B7-H7 in a biological sample comprises contacting the biological sample with an anti-B7-H7 antibody under conditions permissive for binding of the anti-B7-H7 antibody to B7-H7, and detecting whether a complex is formed between the anti-B7-H7 antibody and B7-H7. The methods may include assays known by those of skill in the art, such as Western blot analyses, radioimmunoassays, ELISAs, “sandwich” immunoassays, SPR (e.g., Biacore), immunoprecipitation assays, fluorescent immunoassays, protein A immunoassays, and immunohistochemistry (IHC).

In some embodiments, the anti-B7-H7 antibody is tagged with a detectable label. Useful detectable labels include, but are not limited to, fluorescent molecules, chemiluminescent molecules, bioluminescent molecules, enzymes, and radioisotopes.

The present disclosure provides kits that comprise the B7-H7-binding agents described herein. In some embodiments, a kit is used to perform the methods described herein. In some embodiments, a kit comprises at least one purified B7-H7-binding agent (e.g., an antibody) in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed B7-H7-binding agents of the present disclosure can be readily incorporated into one of the established kit formats that are well known in the art.

### EXAMPLES

### Example 1

### B7-H7 Expression

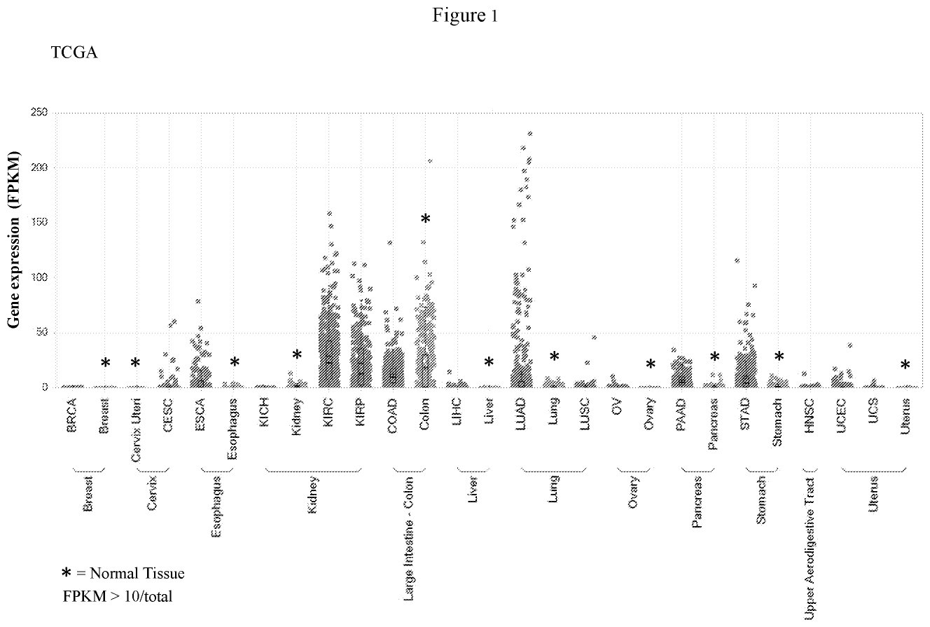

Information from the publicly available database The Cancer Genome Atlas (TCGA) was analyzed to assess B7-H7 mRNA expression in a collection of human cancers and normal tissues. Data from TCGA showed that B7-H7 (HHLA2) mRNA was expressed in tissue from cervical cancer, colon cancer, lung cancer, pancreatic cancer, renal cancer, esophageal cancer, and stomach cancer (FIG. 1). In addition, expression of B7-H7 was observed in normal colon tissue and at very low levels in stomach and pancreas.

### Example 2

### Generation of Anti-B7-H7 Antibodies

Anti-B7-H7 antibodies were generated using the extracellular domain of human B7-H7 as the immunogen. A recombinant construct comprising the extracellular region of human B7-H7 (aa 23-344) and a human Fc sequence was generated and the fusion protein was expressed in mammalian cells. Mice were immunized with the B7-H7-Fc protein and were boosted several times to induce high titers. Blood was drawn from the immunized mice and antibody titers were determined by ELISA and FACS. Single cell suspensions of lymphocytes were obtained from the spleens and lymph nodes of immunized mice that had been determined to have suitably high antibody titers. Lymphocytes were fused with murine myeloma cells by standard methods. Cells were dispersed into 96-well plates in HAT-containing selection media.

### Example 3

### Screening of Antibodies

ELISA assays were used to screen for antibodies that bound to human B7-H7. Antibodies identified by ELISA as binding to human B7-H7 were assayed by FACS for binding to human B7-H7 and cyno B7-H7 expressed on HEK-293T cells. Representative results are shown in Table 10.

TABLE 10

| | Cells expressing | Cells expressing |
|---|---|---|
| Anti-B7-H7 | human B7-H7 | cyno B7-H7 |
| Antibody | (MFI) | (MFI) |
| 1D3 | 247 | 214 |
| 28F5 | 152 | 165 |
| 16D5 | 165 | 199 |
| 2F10 | 101 | 134 |
| 5F7 | 163 | 195 |
| 5B10 | 82 | 93 |
| 5C9 | 185 | 176 |
| 3H6 | 41 | 568 |

### Example 4

### Binding Affinity of Antibodies

The binding affinities of exemplary anti-B7-H7 antibodies to human B7-H7 and cyno B7-H7 were measured using SPR technology (Biacore system, GE Healthcare Life Sciences). Briefly, anti-Fc antibody (Sigma-Aldrich) was immobilized on all four flow cells of a CM5 chip using amine coupling reagents (GE Healthcare LifeSciences). Exemplary antibodies were captured on flow cells 2, 3, and 4 using flow cell 1 as a reference. Human B7-H7 or cyno B7-H7 (samples ranging from 0.62-40 nM) were injected at a flow rate of 50 μL/min at 37° C. Kinetic data were collected over time and fit using the simultaneous global fit equation to yield affinity constants (K_{D} values) for each antibody.

Table 11 shows the binding affinities (K_{D}) for antibodies 2D7, 1D3, 28F5, 16D5, 2F10, 5F7, 5B10, 5C9, and 3H6.

TABLE 11

| Anti-B7-H7 | Human B7-H7 | Cyno B7-H7 |
|---|---|---|
| Antibody | K_{D} (nM) | K_{D} (nM) |
| 2D7 | <0.1 | 0.6 |
| 1D3 | 0.9 | 3.2 |
| 28F5 | 0.4 | 4.5 |
| 16D5 | 0.2 | 6.9 |
| 2F10 | 0.3 | 8.6 |
| 5F7 | 0.6 | 1.2 |
| 5B10 | 4.0 | 4.6 |
| 5C9 | 0.8 | 4.8 |
| 3H6 | 0.8 | 6.9 |

### Example 5

### Antibody Blocking of the Interaction Between B7-H7 and B7-H7 Receptors

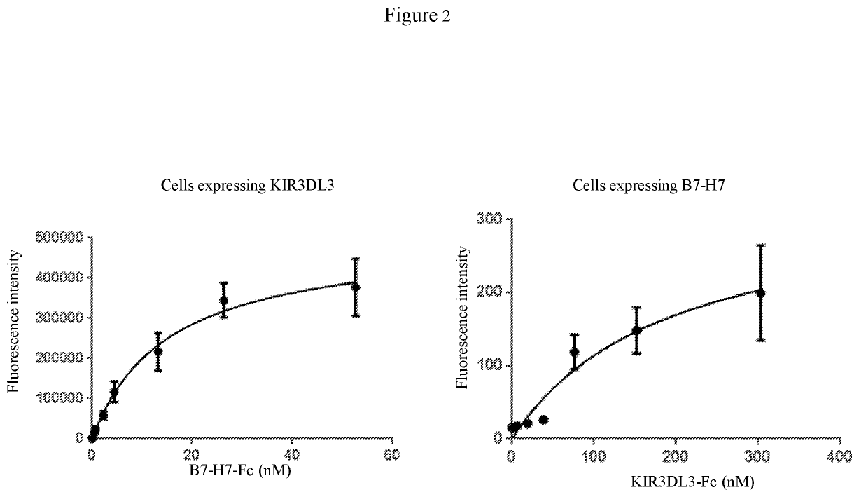

The molecule CD28H has previously been identified as a receptor of B7-H7 (Zhu et al., 2013, Nature Communications, ncomms3043; Zhu et al. refer to B7-H7 as “B7-H5”). Studies by the inventors have identified KIR3DL3 as a second receptor for B7-H7. HEK-293T cells were transiently transfected with a KIR3DL3 construct. 40,000 cells were aliquoted into each well of a 384-well plate and were transfected with 200 ng of plasmid DNA encoding KIR3DL3 using Lipofectamine™ 2000 (ThermoFisher Scientific). After 48 hours, soluble B7-H7-Fc (serially diluted from 10 to 0.16 pg/ml) was added to the wells and plates were incubated from one hour at room temperature. An Alexa Fluor® 488-labeled anti-human Fc secondary antibody was added to the wells and plates were incubated for one hour at room temperature. Cells were washed with HBSS and fixed for 15 minutes with 7.4% formalin in PBS. Cells were washed with PBS and the fluorescence signals were detected using a CellInsight™ platform (ThermoFisher Scientific). A similar experiment was conducted with cells transiently transfected with a B7-H7 construct and soluble biotinylated KIR3DF3 ECD. An Alexa Fluor® 488-labeled streptavidin was used for detection. B7-H7 bound to KIR3DF3 with a K_{D} of 15 nM and KIR3DF3 bound to B7-H7 with a K_{D} of 192 nM (FIG. 2).

Exemplary anti-B7-H7 antibodies identified by ELISA and FACS were tested for their ability to block binding of soluble B7-H7 to receptors KIR3DL3 or CD28H in a FACS assay. HEK-293 cells were transiently transfected with a construct encoding (i) a membrane-bound KIR3DL3 protein or (ii) a membrane-bound CD28H protein. After 48 hours, transfected cells were suspended in ice cold PBS containing 1% FBS and 0.1% sodium azide and incubated on ice in the presence of 1 pg/ml of soluble human B7-H7-Fc protein and antibodies 2D7, 1D3, 28F3, 16D5, 2F10, 5F7, 5B10, 5C9, or 3H6 (serially diluted from 10 pg/ml to 4.6 ng/ml). A second incubation with an anti-human Fc secondary antibody was performed to detect cells bound by the soluble B7-H7-Fc. The cells were analyzed on a BD FACSCalibur instrument (BD Biosciences) and the data was processed using FlowJo software. Calculated IC50s are shown Table 12.

TABLE 12

| | Cells expressing | Cells expressing |
|---|---|---|
| | human KIR3DL3 | human CD28H |
| Anti-B7-H7 | Soluble hB7-H7-Fc | |
| antibody | IC50 (nM) | IC50 (nM) |
| 2D7 | 2.0 | 2.1 |
| 1D3 | 5.0 | 8.0 |
| 28F5 | 2.6 | 2.7 |
| 16D5 | 1.7 | 2.0 |
| 2F10 | 2.5 | 3.0 |
| 5F7 | 1.6 | 1.8 |
| 5B10 | 2.4 | 3.4 |
| 5C9 | 3.0 | 3.4 |
| 3H6 | 2.7 | 2.5 |

These results demonstrated that the exemplary antibodies were able to block binding of hB7-H7 to receptor KIR3DL3 and also to block binding of hB7-H7 to receptor CD28H.

### Example 6

### NK Cell Cytotoxicity Assay

The inventors hypothesized that B7-H7 had the ability to suppress the activity of NK cells when binding to or interacting with KIR3DL3. NKL is a human natural killer lymphoblastic leukemia/lymphoma cell line. A KIR3DL3-expressing NKL cell line was generated by lentiviral transduction. K-562 is a human myelogenous leukemia cell line that is widely used as a target in NK cytotoxicity assays. K-562 cells were transfected with a construct encoding human B7-H7 to produce K-562 cells expressing hB7-H7 on the cell surface referred to herein as “K-562/hB7-H7 cells”. K-562 and K-562-hB7-H7 target cells were labeled with 15 pM calcein-AM (ThermoFisher Scientific). The labeled targets were incubated with the effector NKL-KIR3DL3 cells at a target:effector ratio of 1:10 in the presence of anti-KIR3DL3 or anti-B7-H7 antibodies (10 pg/ml) plus 5 ng/ml of IL2 for 1 hour at 37° C. The amount of calcein-AM released into the supernatant, an indicator of target cell lysis, was measured by a fluorometer (EnSpire® Multimode Plate Reader, PerkinElmer).

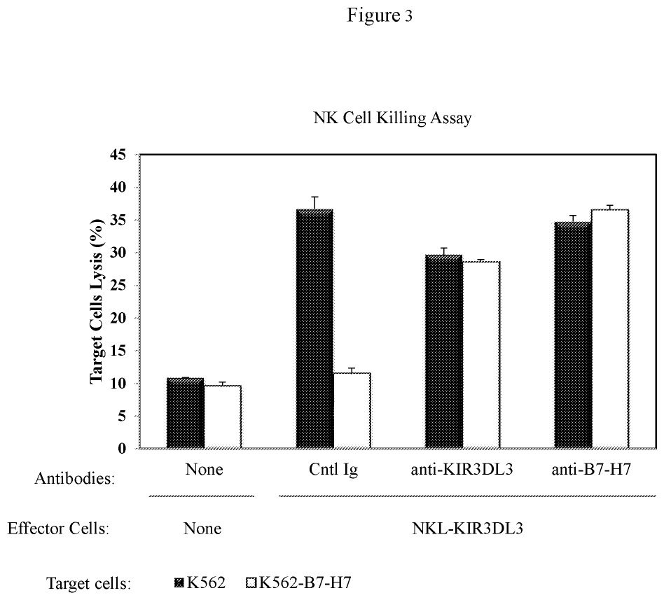

As shown in FIG. 3, the presence of B7-H7 on the surface of the target cells suppressed cytolytic activity of the NKL-KIR3DL3 cells (see control Ig samples). Surprisingly, the anti-KIR3DL3 and anti-B7-H7 antibodies inhibited the suppression of the cytolytic activity. In the presence of the antibodies, the level of K562-B7-H7 target cell lysis was increased to levels equal to lysis of target cells not expressing B7-H7.

In subsequent experiments, anti-B7-H7 antibodies 2D7, 1D3, 28F3, 16D5, 2F10, 5F7, 5B10, 5C9, and 3H6 as well as a control IgG antibody were studied for their effect on the cytotoxic activity of NK cells. The inventors have identified a subset of NK-92 cells (a human natural killer cell line) that spontaneously expresses KIR3DL3 in culture. Cytotoxicity assays using NK92-KIR3DL3 effectors and K562-B7-H7 target cells were performed as described above in the presence of exemplary antibodies (serially diluted 5 to 0.02 pg/ml).

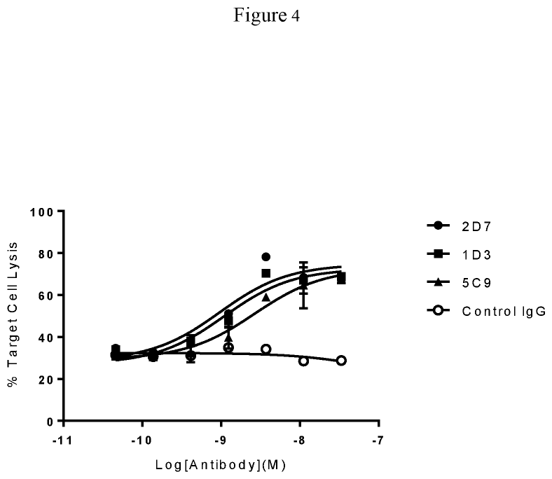

Calculated EC50s and relative efficacies are shown Table 13 and exemplary antibodies 2D7, 1D3, and 5C9 are shown in FIG. 4.

TABLE 13

| | Induction of NK-92 cytotoxicity | |
|---|---|---|
| | EC50 | Relative efficacy |
| Anti-B7-H7Antibody | (nM) | (%) |
| 2D7 | 1.0 | >120 |
| 1D3 | 1.2 | 136 |
| 28F5 | 0.5 | 150 |
| 16D5 | 0.5 | 135 |
| 2F10 | 0.9 | 146 |
| 5F7 | 0.6 | 151 |
| 5B10 | 1.6 | 148 |
| 5C9 | 1.6 | 155 |
| 3H6 | 0.8 | 105 |

These results suggest that the interaction of KIR3DL3 and B7-H7 inhibits and/or suppresses NK cell activity. Importantly, the blockade of B7-H7 with anti-B7-H7 antibodies appears to reduce the B7-H7-associated inhibition of NK activity as seen by the increase in NK cytotoxicity in the presence of the antibodies. These data support the theory that inhibition or blockade of B7-H7 can “release the brake” on NK cell suppression and enhance immune responses.

### Example 7

### Tetanus Toxoid Recall Assay

Antigens, such as tetanus toxoid, are widely used by those of skill in the art to measure antigen-specific recall T-cell responses when assessing cellular immunity. An antigen-specific recall assay measures the ability of T-cells to respond to a specific antigen after that antigen has been processed and presented by antigen-presenting cells. Anti-B7-H7 antibody 2D7, an anti-PD-1 antibody, an anti-PD-L1 antibody, an anti-CD28H antibody, and a control antibody were studied for their effect in a tetanus toxoid recall assay. Cryopreserved human peripheral blood mononuclear cells (PBMC) were thawed in RPMI medium with 10% FBS, washed, and resuspended to 5×10⁶ cells/mL. 100 μL of cells was incubated with 50 μL of antibodies for 60 minutes at 37° C. Tetanus toxoid (Astarte Biologies) was added to a final concentration of 2 pg/mL and the cells were incubated for 7 days. Supernatants from each sample were analyzed for IFN-γ and TNF-alpha production using a Bio-Plex® Multiplex Immunoassay kit (Fife Science Research).

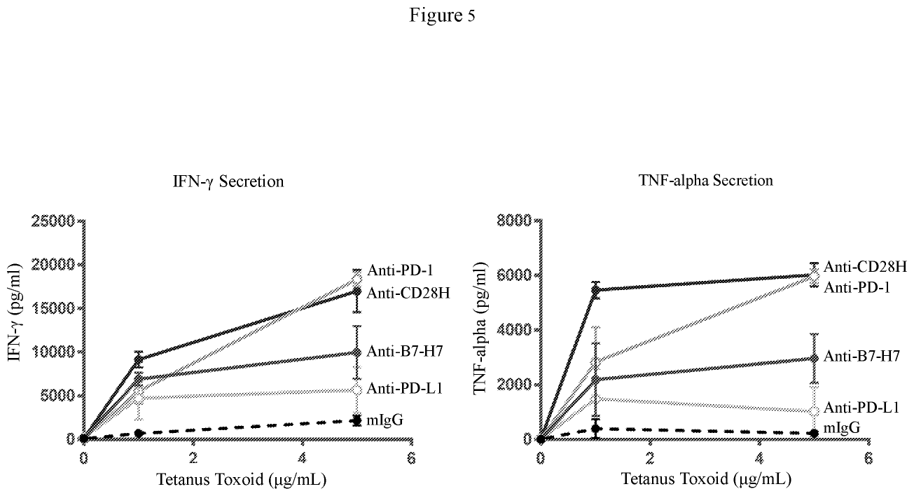

As shown in FIG. 5, the production of IFN-γ and TNF-alpha was increased by the blockade of B7-H7. This effect was also seen with other checkpoint inhibitors, such as anti-PD-1 and anti-PD-L1 antibodies.

These data further support the theory that inhibition or blockade of B7-H7 can enhance immune responses.

### Example 8

### Additional Characterization of Anti-B7-H7 Antibodies

Antibodies identified by ELISA and FACS as binding to B7-H7 were further characterized in binning experiments as described herein. Briefly, epitope binning assays were performed by directly coating wells of a 96-well plate with a first antibody. Non-specific binding was inhibited by incubating the plates with blocking solution (PBS/1% BSA). Test antibodies were pre-incubated with B7-H7 ECD-Fc protein for 30 minutes, added to individual wells, and incubated for one hour at room temperature. After washing the plates three times, HRP-anti-human Fc secondary antibody was added to the wells and the plates were incubated for one hour at room temperature. After washing the plates three times, TMB (tetramethyl benzidine) was added to the plates for detection. A positive signal indicates that the first antibody, captured on the plate, was able to bind B7-H7 ECD-Fc protein and therefore the first antibody and the second antibody do not compete for binding to B7-H7. A negative signal indicates that the first antibody, captured on the plate, was not able to bind B7-H7 ECD-Fc protein and therefore the first antibody and the second antibody compete for binding to B7-H7. Antibodies 2D7, 2F10, 3H6, 5F7, 16D5 and 28F5 were determined to belong to the same bin (Bin 1) as they competed with each other in the assay. Antibodies 1D3 and 5B10 were determined to belong to the same bin (Bin 2). Antibody 5C9 was determined to belong to a separate bin (Bin 3) as it did not compete with the antibodies of Bin 1 or Bin 2.

Antibodies 2D7, 2F10, 3H6, 5F7, 16D5, 28F5, 1D3, 5B10, and 5C9 were sequenced and the heavy chain variable region and light chain variable region amino acid sequences are disclosed herein and summarized in Table 14.

TABLE 14

| | Heavy Chain | Light Chain |
|---|---|---|
| Antibody | Variable Region | Variable Region |
| 2D7 | SEQ ID NO: 19 | SEQ ID NO: 20 |
| 2F10 | SEQ ID NO: 32 | SEQ ID NO: 33 |
| 3H6 | SEQ ID NO: 36 | SEQ ID NO: 37 |
| 5F7 | SEQ ID NO: 41 | SEQ ID NO: 42 |
| 16D5 | SEQ ID NO: 47 | SEQ ID NO: 48 |
| 28F5 | SEQ ID NO: 55 | SEQ ID NO: 56 |
| 1D3 | SEQ ID NO: 63 | SEQ ID NO: 64 |
| 5B10 | SEQ ID NO: 67 | SEQ ID NO: 68 |
| 5C9 | SEQ ID NO: 75 | SEQ ID NO: 76 |

The heavy chain and light chain variable region CDRs for the individual antibodies are disclosed in Tables 1-9 herein.

### Example 9

### Pharmacokinetic Analysis of Anti-B7-H7 Antibodies

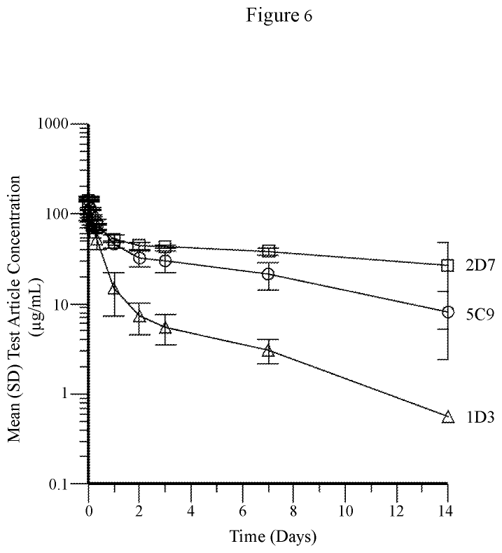

CD-1 mice were injected intravenously with a single dose of antibody 2D7, 1D3, or 5C9. These antibodies were chimeric versions comprising the murine heavy chain and light chain variable regions and a human Fc region. Blood samples from the injected mice were analyzed at multiple time points, up to 14 days post injection. Antibody concentration was determined by ELISA using an anti-idiotype antibody. While all three antibodies showed a similar maximum concentration (Cmax), these antibodies were cleared at different rates as shown in Table 15 and FIG. 6.

TABLE 15

| | CL |
|---|---|
| Antibody | (mL/day/kg) |
| 2D7 | 7.7 |
| 5C9 | 12 |
| 1D3 | 54 |

The pharmacokinetic analysis supports a dosing regimen of a single injection every two weeks or even longer for anti-B7-H7 antibody 2D7.

### Example 10

### Humanized Antibody

Based on the antibody characterization data as well as additional studies, antibody 2D7 was selected for humanization. Antibody 2D7 was humanized by methods known by those skilled in the art and is referred to herein as Hz2D7. The heavy chain variable sequence of Hz2D7 is SEQ ID NO:21 and the light chain variable sequence of Hz2D7 is SEQ ID NO:22. The CDR sequences of Hz2D7 are listed in Table IB.

The binding affinity of Hz2D7 to human B7-H7 and cyno B7-H7 was determined using SPR technology (Biacore system) as described herein and compared with the binding affinity of the parental antibody and with a chimeric 2D7 antibody comprising the murine heavy chain and light chain variable regions of 2D7 and a human Fc region.

TABLE 16

| | Human B7-H7 | | Cyno B7-H7 | |
|---|---|---|---|---|
| Antibody | K_{D} (nM) | K_{off} (1/s) | K_{D} (nM) | K_{off} (1/s) |
| 2D7 | <0.1 | 1.00 × 10⁻⁵ | 0.65 | 1.10 × 10⁻⁴ |
| Chimeric 2D7 | <0.1 | 5.61 × 10⁻⁶ | 0.14 | 3.50 × 10⁻⁵ |
| Hz2D7 | <0.1 | 1.01 × 10⁻⁵ | 0.40 | 7.60 × 10⁻⁵ |

As shown in Table 16, Hz2D7 exhibited binding to both human and cyno B7-H7 at a level similar to the parental antibody.

### Example 11

### Inhibition of Tumor Growth in a Mouse Model

A humanized mouse model was used to study the effect of an anti-B7-H7 antibody on tumor growth. Humanized NSG™ mice were obtained from The Jackson Laboratories. Each mouse was injected subcutaneously with AsPC1 cells (1×10⁶ cells/mouse). The AsPC1 cell line was derived from the ascites of a patient with pancreatic cancer. Tumors were allowed to grow until they reached an average volume of approximately 75 mm³. Tumor-bearing mice were randomized into 4 groups (n=5 mice per group). Tumor-bearing mice were treated with either a control anti-KLH antibody (20 mg/kg), anti-B7-H7 antibody Hz2D7 (20 mg/kg), an anti-PD-1 antibody (5 mg/kg), or a combination of Hz2D7 and anti-PD-1 antibody. Antibodies were dosed intravenously once a week. Tumor growth was monitored and tumor volumes were measured with electronic calipers at the indicated time points.

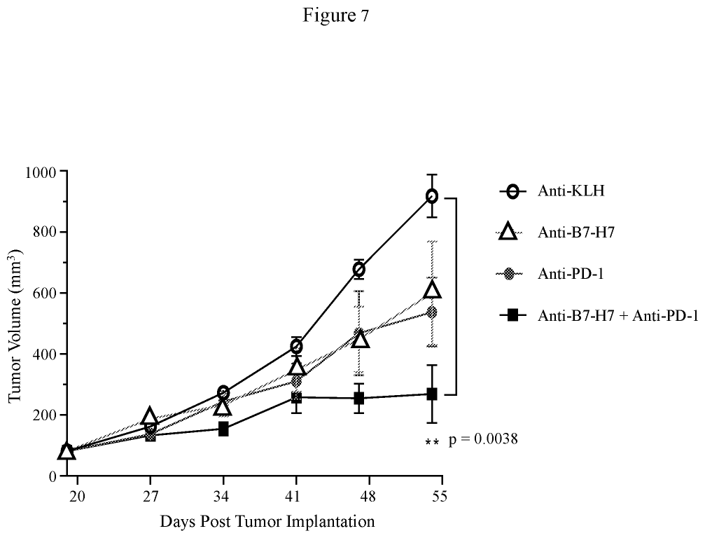

As shown in FIG. 7, tumor growth was inhibited in the mice treated with the Hz2D7 antibody as compared to control. The inhibition of tumor growth was similar to what was observed in mice treated with an anti-PD-1 antibody. Surprisingly, the combination of Hz2D7 and anti-PD-1 antibody inhibited tumor growth to a significantly greater level than either Hz2D7 alone or anti-PD-1 alone.

### Example 12

### Analysis of Anti-B7-H7 Antibody in Cynomolgus Monkeys

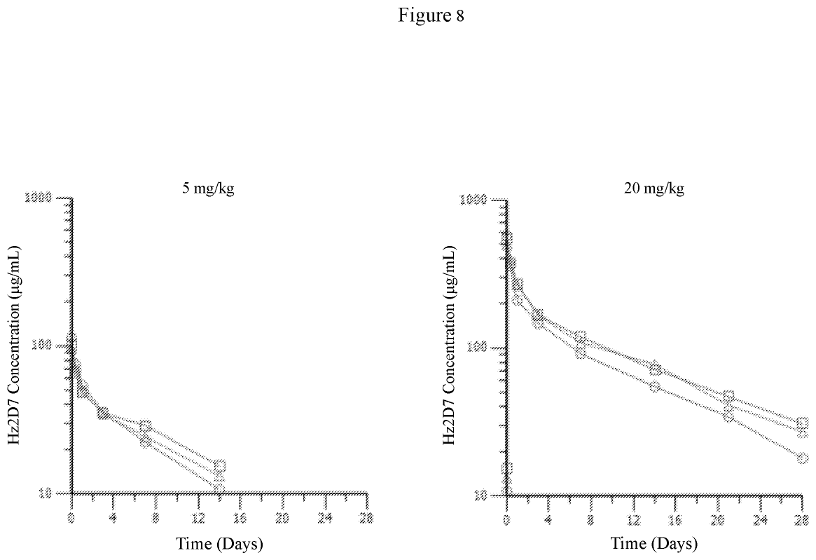

Cynomolgus monkeys (male; n=3) were injected intravenously with a single dose of antibody Hz2D7 at 5 mg/kg or 20 mg/kg. Blood samples from the injected animals were analyzed at multiple time points, up to 14 days post injection. Antibody concentration was determined by ELISA using an anti-idiotype antibody. The Hz2D7 antibody had a clearance value of 7 mL/kg/day (FIG. 8) and the terminal half-life was calculated to be 9.8±1.5 days. These results suggest that a dosing regimen of once every 3 weeks or once every 4 weeks is feasible.

There were no clinical signs of toxicity (e.g., hematology parameters such as RBCs, WBCs, lymphocytes, monocytes, and neutrophils were unchanged) and no decrease in body weight for up to four weeks following a single dose of 20 mg/kg.

In addition, no test agent-related changes in regard to gross pathology, organ weights, or histopathology were observed in monkeys treated with the antibody at either concentration. The tissues examined included bone marrow, esophagus, gall bladder, gut-associated lymphoid tissue, heart, intestine (colon, ileum, rectum, cecum, duodenum, jejunum), kidney, liver, lung, pancreas, spleen, lymph nodes (mesenteric and axillary) and stomach.

Although the foregoing present disclosure has been described in some detail by way of illustration and example for purposes of clarity of understanding, the descriptions and examples should not be construed as limiting the scope of the present disclosure. The embodiments of the present disclosure described herein are intended to be merely exemplary, and those skilled in the art will recognize numerous equivalents to the specific procedures described herein. All such equivalents are considered to be within the scope of the present disclosure and are covered by the embodiments.

All publications, patents, patent applications, internet sites, and accession numbers/database sequences including both polynucleotide and polypeptide sequences cited herein are hereby incorporated by reference in their entirety for all purposes to the same extent as if each individual publication, patent, patent application, internet site, or accession number/database sequence were specifically and individually indicated to be so incorporated by reference.

Following are sequences disclosed in the application with the exception of the heavy chain and light chain variable region CDR sequences defined in Tables 1-9.

Human B7-H7 amino acid sequence with predicted signal
sequence underlined
(SEQ ID NO: 1)
MKAQTALSFFLILITSLSGSQGIFPLAFFIYVPMNEQIVIGRLDEDIILPSSFERGSEVV
IHWKYQDSYKVHSYYKGSDHLESQDPRYANRTSLFYNEIQNGNASLFFRRVSLLDEGIYT
CYVGTAIQVITNKVVLKVGVFLTPVMKYEKRNINSFLICSVLSVYPRPIITWKMDNIPIS
ENNMEETGSLDSFSINSPLNITGSNSSYECTIENSLLKQTWTGRWTMKDGLHKMQSEHVS
LSCQPVNDYFSPNQDFKVTWSRMKSGTFSVLAYYLSSSQNTIINESRFSWNKELINQSDF
SMNLMDLNLSDSGEYLCNISSDEYTLLTIHTVHVEPSQETASHNKGLWILVPSAILAAFL
LIWSVKCCRAQLEARRSRHPADGAQQERCCVPPGERCPSAPDNGEENVPLSGKV
Human B7-H7 amino acid sequence without predicted signal
sequence
(SEQ ID NO: 2)
IFPLAFFIYVPMNEQIVIGRLDEDIILPSSFERGSEVVIHWKYQDSYKVHSYYKGSDHLE
SQDPRYANRTSLFYNEIQNGNASLFFRRVSLLDEGIYTCYVGTAIQVITNKVVLKVGVFL
TPVMKYEKRNINSFLICSVLSVYPRPIITWKMDNIPISENNMEETGSLDSFSINSPLNIT
GSNSSYECTIENSLLKQTWTGRWTMKDGLHKMQSEHVSLSCQPVNDYFSPNQDFKVTWSR
MKSGTFSVLAYYLSSSQNTIINESRFSWNKELINQSDFSMNLMDLNLSDSGEYLCNISSD
EYTLLTIHTVHVEPSQETASHNKGLWILVPSAILAAFLLIWSVKCCRAQLEARRSRHPAD
GAQQERCCVPPGERCPSAPDNGEENVPLSGKV
Human B7-H7 extracellular domain
(SEQ ID NO: 3)
IFPLAFFIYVPMNEQIVIGRLDEDIILPSSFERGSEVVIHWKYQDSYKVHSYYKGSDHLE
SQDPRYANRTSLFYNEIQNGNASLFFRRVSLLDEGIYTCYVGTAIQVITNKVVLKVGVFL
TPVMKYEKRNINSFLICSVLSVYPRPIITWKMDNIPISENNMEETGSLDSFSINSPLNIT
GSNSSYECTIENSLLKQTWTGRWTMKDGLHKMQSEHVSLSCQPVNDYFSPNQDFKVTWSR
MKSGTFSVLAYYLSSSQNTIINESRFSWNKELINQSDFSMNLMDLNLSDSGEYLCNISSD
EYTLLTIHTVHVEPSQETASHNK
Human B7-H7 IgV-type domain 1
(SEQ ID NO: 4)
IHWKYQDSYKVHSYYKGSDHLESQDPRYANRTSLFYNEIQNGNASLFFRRVSLLDEGIYT
CYVGTAIQVIT
Human B7-H7 IgC1-type domain
(SEQ ID NO: 5)
VGVFLTPVMKYEKRNINSFLICSVLSVYPRPIITWKMDNITISENNMEETGSLDSFSINS
PLNITGSNSSYECTIENSLLKQTWT
Human B7-H7 IgV-type domain 2
(SEQ ID NO: 6)
QSEHVSLSCQPVNDYFSPNQDFKVTWSRMKSGTFSVLAYYLSSSQNTIINESRFSWNKEL
INQSDFSMNLMDLNLSDSGEYLCNISSDEYTLLT
Cynomolgus monkey B7-H7 amino acid sequence with signal
sequence underlined
(SEQ ID NO: 7)
MKAQTSFFLILISSLSGSQGIFLSAFFTYVPMNEQIIIGRLGEDIILPSSFERGSEVVIH
WKYQDSYNSYNVHSYYKGSGRLESQDTRYANRTSLFYNEIQNGNASLFFRRLSLLDEGIY
TCYVGTAIQAITNKVVLKVGVFLTPMMKYEKRNINSFLICNVLSVYPRPIITWKMDNTPI
SENNMQETGSLGPFSINSTLNITGSNSSYECTIENSLLKQTWTGRWTMKDGLHKMQSEHV
SLSCELVNDYFSPNQDFKVTWSRMESGISSILAYYLSSSQNTIFYESRFSWNKELKNQSD
FSMNLTDLSLSDSGEYLCNISSDEYTLLTIHTVHVEPSQETASDNKVLWILVASLILVLC
LIWLIWKVKCCTERRRVSTESTHDDNASDNAEENMRLSGRVQQMREDLDNS
Cynomolgus monkey B7-H7 amino acid sequence without
predicted signal sequence
(SEQ ID NO: 8)
IFLSAFFTYVPMNEQIIIGRLGEDIILPSSFERGSEVVIHWKYQDSYNSYNVHSYYKGSG
RLESQDTRYANRTSLFYNEIQNGNASLFFRRLSLLDEGIYTCYVGTAIQAITNKVVLKVG
VFLTPMMKYEKRNINSFLICNVLSVYPRPIITWKMDNITISENNMQETGSLGPFSINSTL
NITGSNSSYECTIENSLLKQTWTGRWTMKDGLHKMQSEHVSLSCELVNDYFSPNQDFKVT
WSRMESGISSILAYYLSSSQNTIFYESRFSWNKELKNQSDFSMNLIDLSLSDSGEYLCNI
SSDEYILLTIHTVHVEPSQETASDNKVLWILVASLILVLCLIWLIWKVKCCTERRRVSTE
STHDDNASDNAEENMRLSGRVQQMREDLDNS
Cynomolgus monkey B7-H7 extracellular domain
(SEQ ID NO: 9)
IFLSAFFTYVPMNEQIIIGRLGEDIILPSSFERGSEVVIHWKYQDSYNSYNVHSYYKGSG
RLESQDTRYANRTSLFYNEIQNGNASLFFRRLSLLDEGIYTCYVGTAIQAITNKVVLKVG
VFLTPMMKYEKRNINSFLICNVLSVYPRPIITWKMDNITISENNMQETGSLGPFSINSTL
NITGSNSSYECTIENSLLKQTWTGRWTMKDGLHKMQSEHVSLSCELVNDYFSPNQDFKVT
WSRMESGISSILAYYLSSSQNTIFYESRFSWNKELKNQSDFSMNLIDLSLSDSGEYLCNI
SSDEYTLLTIHTVHVEPSQETASDNK
Cynomolgus monkey B7-H7 IgV-type domain 1
(SEQ ID NO: 10)
IHWKYQDSYNSYNVHSYYKGSGRLESQDTRYANRTSLFYNEIQNGNASLFFRRLSLLDEG
IYTCYVGTAIQAIT
Cynomolgus monkey B7-H7 IgC1-type domain
(SEQ ID NO: 11)
VGVFLTPMMKYEKRNINSFLICNVLSVYPRPIITWKMDNITISENNMQETGSLGPFSINS
TLNITGSNSSYECTIENSLLKQTWT
Cynomolgus monkey B7-H7 IgV-type domain 2
(SEQ ID NO: 12)
QSEHVSLSCELVNDYFSPNQDFKVTWSRMESGISSILAYYLSSSQNTIFYESRFSWNKEL
KNQSDFSMNLTDLSLSDSGEYLCNISSDEYTLLT
2D7 Heavy chain variable region amino acid sequence
(SEQ ID NO: 19)
EVQLQQSGPELVKPGASVKISCKTSGYTFTEYTMHWVKQSHGKSLEWIGGINPNNYGAPY
NQKFKGKATLTVDKSSNTAYMELRSLTSEDSAIYYCASGGYYFDYWGQGTTLIVSS
2D7 Light chain variable region amino acid sequence
(SEQ ID NO: 20)
DIVMTQSHKFMSTSFGDRVSITCKASQDVGTAVAWYQQKLGQSPKLLISWAFTRHTGVPD
RFTGSGSGTDYTLTISSLQAEDLALYYCQQHYDTPFTFGSGTKLEIK
Hz2D7 Heavy chain variable region amino acid sequence
(SEQ ID NO: 21)
QVQLVQSGAEVKKPGASVKVSCKTSGYTFTEYTMHWVRQAPGQGLEWMGGINPNNYGAPY
NQKFKGRVTMTVDTSISTAYMELSRLRSDDTAVYYCASGGYYFDYWGQGTLVTVSS
Hz2D7 Light chain variable region amino acid sequence
(SEQ ID NO: 22)
DIQMTQSPSSLSASVGDRVTITCKASQDVGTAVAWYQQKPGKAPKLLIYWAFTRHTGVPS
RFSGSGSGTDYTLTISSLQPEDFATYYCQQHYDTPFTFGQGTKVEIK
Hz2D7 Heavy chain amino acid sequence with signal
sequence underlined
(SEQ ID NO: 23)
MDMRVPAQLLGLLLLWLRGARCQVQLVQSGAEVKKPGASVKVSCKTSGYTFTEYTMHWVR
QAPGQGLEWMGGINPNNYGAPYNQKFKGRVTMTVDTSISTAYMELSRLRSDDTAVYYCAS
GGYYFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVICVVVDVSHEDPEVKFNWYV
DGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
Hz2D7 Heavy chain amino acid sequence without signal sequence
(SEQ ID NO: 24)
QVQLVQSGAEVKKPGASVKVSCKTSGYTFTEYTMHWVRQAPGQGLEWMGGINPNNYGAPY
NQKFKGRVTMTVDTSISTAYMELSRLRSDDTAVYYCASGGYYFDYWGQGTLVTVSSASTK
GPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGN
VFSCSVMHEALHNHYTQKSLSLSPGK
Hz2D7 Light chain amino acid sequence with signal
sequence underlined
(SEQ ID NO: 25)
MDMRVPAQLLGLLLLWLRGARCDIQMTQSPSSLSASVGDRVTITCKASQDVGTAVAWYQQ
KPGKAPKLLIYWAFTRHTGVPSRFSGSGSGTDYTLTISSLQPEDFATYYCQQHYDTPFTF
GQGTKVEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Hz2D7 Light chain amino acid sequence without signal sequence
(SEQ ID NO: 26)
DIQMTQSPSSLSASVGDRVTITCKASQDVGTAVAWYQQKPGKAPKLLIYWAFTRHTGVPS
RFSGSGSGTDYTLTISSLQPEDFATYYCQQHYDTPFTFGQGTKVEIKRTVAAPSVFIFPP
SDEQLKSGTASVVCLLNNFYPREAKVQWKVDNALQSGNSQESVTEQDSKDSTYSLSSILT
LSKADYEKHKVYACEVTHQGLSSPVTKSFNRGEC
2F10 Heavy chain variable region amino acid sequence
(SEQ ID NO: 32)
EIQLQQTGPELVKPGASLRISCKASGYSFTDYIIVWVKQNQGKNLDWIEKINPYYGITTY
NLRFEDKATLTVNKSSTTAYMQLNSLTSEDSAVYYCARWDYVSTLFAMDYWGQGTSITVS
S
2F10 Light chain variable region amino acid sequence
(SEQ ID NO: 33)
DIVMTQSHKVMSTSVGDRVSIICKASQDVGTAVAWYQQKPGQSPKLLIYWASTRHTGVPD
RFTGSGSGTDFILTITNVQSENLANYFCQQYKRYYTFGGGTKLEIK
3H6 Heavy chain variable region amino acid sequence
(SEQ ID NO: 36)
EVQLQQSGPELVKPGASVKISCKTSGYTFTEYTMHWVKQSHGKSLEWIGGINPNNGGAPY
NQKFKGKATLTVDKSSSTAYMELRSLTSEDSAVYYCARGGYYFDYWGQGTTLTVSS
3H6 Light chain variable region amino acid sequence
(SEQ ID NO: 37)
DIVMTQSHKFMSTSVGDRVSITCKASQDVSTAVAWYQQKSGQSPKLLIYWASTRHTGVPD
RFTGSGSGTDYTLTISSVQAEDLALYYCQQHYDTPFTFGGGTKLEIK
5F7 Heavy chain variable region amino acid sequence
(SEQ ID NO: 41)
QEQLQQPGTELVKPGASVKISCKASGYTFSSYSMHWVKLTPGQGLEWMGTIYPGNENTSY
NQKFKGKATLTADKSSSTAYMQLSSLTSEDSAVYYCARGGYYFDYWGQGTTLTVSS
5F7 Light chain variable region amino acid sequence
(SEQ ID NO: 42)
DIVMTQSHKFLSTSVGDRVSITCKASQDVSTAVAWYQQKPGQSPKLLIYWASTRHTGVSD
RFTGSGSGTDFTLTISSVQAEDLALYYCQQHFDIPYWFGGGTKLEIK
16D5 Heavy chain variable region amino acid sequence
(SEQ ID NO: 47)
QVQLQQSAAELARPGASVKMSCKASGYTFTTYTMHWVKQRPGQGLEWIGYINPSRGYSDY
SQKFQGKSTLTTDKSSNTAYIQLTSLTSEDSAVYFCARGGYDFDYWGQGTTLTVSS
16D5 Light chain variable region amino acid sequence
(SEQ ID NO: 48)
DIVMTQSHKFMSTSVGDRVSITCKASQDVGTAVAWYQQKPGQSPKLLLYWASTRHTGVPD
RFTGSGSGTDYTLTISSVQAEDLALYHCQQHFITPYTFGGGTKLEIK
28F5 Heavy chain variable region amino acid sequence
(SEQ ID NO: 55)
QVQLKESGPGLVAPSQSLSITCTVSGFSLTGYGVNWVRQPPGKGLEWLGVIWGDGSTDYN
SVLKSRLSISKDNSKSQVFLKMNSLQTDDTARYYCAREATEYLYWYFDVWGAGTTVTVSS
28F5 Light chain variable region amino acid sequence
(SEQ ID NO: 56)
DIVLTQSPASLAVSLGQRATISCRASESVEYYGSSLMQWYQQKPGQPPKLLIFAASNVES
GVPPRFSGSGSGTDFNLNIHPVDEDDIAMYFCQQGRRVPWTFGGGTKLEIK
1D3 Heavy chain variable region amino acid sequence
(SEQ ID NO: 63)
EVQLQQSGPELEKPGASVKISCKASGYSFTGYNMNWVKESNGRSLEWIGNIDPYSGGSTY
NQKFKGKATMTVDKSSSTAYMQLKSLTSEDSAVYYCARSVYDAPWLAHWGQGTLVTVSA
1D3 Light chain variable region amino acid sequence
(SEQ ID NO: 64)
DIQMTQSPASLSASVGETVTITCRASENIYIYLAWYQQKQGKSPQLLVYNAKTLAEGVPS
RFSGSGSGTQFSLKINRLQPEDFGNYYCQHHYGTPPTFGGGTKLEIK
5B10 Heavy chain variable region amino acid sequence
(SEQ ID NO: 67)
EVQLQQSGPELEKPGASVKISCKASGYSFTGYNMNWVKQSNGKSLEWIGNIDPYSGGSTY
NQKFKGKATLTVDKSSSTASLQLKSLTSEDSSVYYCARSFYDAPYLTYWGQGTLVTVSA
5B10 Light chain variable region amino acid sequence
(SEQ ID NO: 68)
DIQMTQSPTSLSASVGETVTITCRASENIFIYLAWYQQKQGKSPQLLVFNAKTLAEGVPS
RFSGSGSGTQFSLKINSLRPEDFGTYYCQHHYGTPPTFGGGTKLEV
5C9 Heavy chain variable region amino acid sequence
(SEQ ID NO: 75)
QVQLQQSGAELAKPGASVKMSCKASGYTFTSFWIHWIKQRPGQGLEWIGYIIPNTDYTEY
NQKFKDKATFTADKSSSTAYMQLSSLTSEDSAVFYCARGLRGAYYFDYWGQGTTLTVSS
5C9 Light chain variable region amino acid sequence
(SEQ ID NO: 76)
DIVLTQSPASLAVSLGLRATISCRSSQSVSTSTNGYMHWYQQKPGQPPKLLIMYASNLES
GVPARFSGSGSGTDFTLNIHPVDEEDTATYYCQHSWVLPYTFGGGTKLEIK
Human IgG1 constant region
(SEQ ID NO: 77)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region E233A/L235A
(SEQ ID NO: 78)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPALAGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region L234A/L235A
(SEQ ID NO: 79)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 constant region L234A/L235A/P329G
(SEQ ID NO: 80)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human CD28H
(SEQ ID NO: 81)
MGSPGMVLGLLVQIWALQEASSLSVQQGPNLLQVRQGSQATLVCQVDQATAWERLRVKWT
KDGAILCQPYITNGSLSLGVCGPQGRLSWQAPSHLTLQLDPVSLNHSGAYVCWAAVEIPE
LEEAEGNITRLFVDPDDPTQNRNRIASFPGFLFVLLGVGSMGVAAIVWGAWFWGRRSCQQ
RDSGNSPGNAFYSNVLYRPRGAPKKSEDCSGEGKDQRGQSISISFPQPAPRQPHLASRP
CPSPRPCPSPRPGHPVSMVRVSPRPSPTQQPRPKGFPKVGEE
Human CD28H Extracellular domain (aa 23-150)
(SEQ ID NO: 82)
LSVQQGPNLLQVRQGSQATLVCQVDQATAWERLRVKWTKDGAILCQPYITNGSLSLGVCG
PQGRLSWQAPSHLTLQLDPVSLNHSGAYVCWAAVEIPELEEAEGNITRLFVDPDDPTQNR
NRIASFPG
Human KIR3DL3
(SEQ ID NO: 83)
MSLMVVSMACVGFFLLEGPWPHVGGQDKPFLSAWPGTVVSEGQHVTLQCRSRLGFNEFSL
SKEDGMPVPELYNRIFRNSFLMGPVTPAHAGTYRCCSSHPHSPTGWSAPSNPVVIMVTGV
HRKPSLLAHPGPLVKSGETVILQCWSDVRFERFLLHREGITEDPLRLVGQLHDAGSQVNY
SMGPMTPALAGTYRCFGSVTHLPYELSAPSDPLDIVVVGLYGKPSLSAQPGPTVQAGENV
TLSCSSRSLFDIYHLSREAEAGELRLTAVLRVNGTFQANFPLGPVTHGGNYRCFGSFRAL
PHAWSDPSDPLPVSVTGNSRHLHVLIGTSVVTIPFAILLFFLLHRWCANKKNAVVMDQEP
AGNRTVNREDSDEQDPQEVTYAQLNHCVFTQRKITRPSQRPKTPPTDTSV
Human KIR3DL3 Extracellular domain (26-322)
(SEQ ID NO: 84)
QDKPFLSAWPGTVVSEGQHVTLQCRSRLGFNEFSLSKEDGMPVPELYNRIFRNSFLMGPV
TPAHAGTYRCCSSHPHSPTGWSAPSNPVVIMVTGVHRKPSLLAHPGPLVKSGETVILQCW
SDVRFERFLLHREGITEDPLRLVGQLHDAGSQVNYSMGPMTPALAGTYRCFGSVTHLPYE
LSAPSDPLDIVVVGLYGKPSLSAQPGPTVQAGENVTLSCSSRSLFDIYHLSREAEAGELR
LTAVLRVKGTFQANFPLGPVTHGGNYRCFGSFRALPHAWSDPSDPLPVSVTGNSRKL

What is claimed:

1. A binding agent that specifically binds human B7-H7, which comprises:
(a) a heavy chain variable region CDR1 comprising GYTFTEYTMH (SEQ ID NO: 13), a heavy chain variable region CDR2 comprising GINPNNYGAPYNQKFKG (SEQ ID NO: 14), and a heavy chain variable region CDR3 comprising GGYYFDY (SEQ ID NO: 15); and/or
(b) a light chain variable region CDR1 comprising KASQDVGTAVA (SEQ ID NO: 16), a light chain variable region CDR2 comprising WAFTRHT (SEQ ID NO: 17), and a light chain variable region CDR3 comprising QQHYDTPFT (SEQ ID NO: 18).

2. The binding agent of claim 1, which comprises:
(a) a heavy chain variable region having at least 80% sequence identity to SEQ ID NO: 19 or SEQ ID NO:21; and/or
(b) a light chain variable region having at least 80% sequence identity to SEQ ID NO:20 or SEQ ID NO:22.

3. The binding agent of claim 1, which comprises a heavy chain variable region having at least 90% sequence identity to SEQ ID NO: 19.

4. The binding agent of claim 1, which comprises a light chain variable region having at least 90% sequence identity to SEQ ID NO:20.

5. The binding agent of claim 1, which comprises a heavy chain variable region having at least 90% sequence identity to SEQ ID NO:21.

6. The binding agent of claim 1, which comprises a light chain variable region having at least 90% sequence identity to SEQ ID NO:22.

7. The binding agent of claim 1, which comprises a heavy chain variable region having at least 90% sequence identity to SEQ ID NO: 19 and a light chain variable region having at least 90% sequence identity to SEQ ID NO:20.

8. The binding agent of claim 1, which comprises a heavy chain variable region having at least 90% sequence identity to SEQ ID NO:21 and a light chain variable region having at least 90% sequence identity to SEQ ID NO:22.

9. The binding agent of claim 1, which comprises a heavy chain variable region comprising SEQ ID NO: 19 and a light chain variable region comprising SEQ ID NO:20.

10. The binding agent of claim 1, which comprises a heavy chain variable region comprising SEQ ID NO:21.

11. The binding agent of claim 1, which comprises a light chain variable region comprising SEQ ID NO:22.

12. The binding agent of claim 1, which comprises a heavy chain variable region comprising SEQ ID NO:21 and a light chain variable region comprising SEQ ID NO:22.

13. A binding agent that specifically binds human B7-H7, which comprises a CDR1, CDR2, and CDR3 from a heavy chain variable region having the amino acid sequence of SEQ ID NO: 19 and a CDR1, CDR2, and CDR3 from a light chain variable region having the amino acid sequence of SEQ ID NO:20.

14. A binding agent that specifically binds human B7-H7, which comprises a CDR1, CDR2, and CDR3 from a heavy chain variable region having the amino acid sequence of SEQ ID NO:21 and a CDR1, CDR2, and CDR3 from a light chain variable region having the amino acid sequence of SEQ ID NO:22.

15. The binding agent of any one of claims 1-14, which is an antibody.

16. The binding agent of any one of claims 1-15, which is a monoclonal antibody.

17. The binding agent of any one of claims 1-16, which is a chimeric antibody.

18. The binding agent of any one of claims 1-8 or 10-16, which is a humanized antibody.

19. The binding agent of any one of claims 1-18, which is a bispecific antibody or a multispecific antibody.

20. The binding agent of any one of claims 1-19, which is an antibody fragment comprising at least one antigen-binding site.

21. The binding agent of claim 15, which is a Fab, Fab′, F(ab′)₂, Fv, scFv, (scFv)₂, single chain antibody, dual variable region antibody, single variable region antibody, a diabody, or a nanobody.

22. The binding agent of any one of claims 15-19, which is an IgG1 antibody.

23. The binding agent of any one of claims 15-19, which is an IgG2 antibody.

24. The binding agent of any one of claims 15-19, which is an IgG4 antibody.

25. The binding agent of any one of claims 15-24, which comprises a kappa light chain.

26. The binding agent of any one of claims 15-24, which comprises a lambda light chain.

27. A binding agent that specifically binds human B7-H7, which comprises a heavy chain comprising the amino acid sequence of SEQ ID NO:24 and a light chain comprising the amino acid sequence of SEQ ID NO:26.

28. The binding agent of claim 27, which is an antibody.

29. The binding agent of claim 27, which is a monoclonal antibody.

30. The binding agent of any one of claims 27-29, which is a bispecific antibody or a multispecific antibody.

31. A binding agent that specifically binds human B7-H7, wherein the binding agent comprises a scaffold protein and a heavy chain variable region CDR1, CDR2, and CDR3 from a heavy chain variable region comprising SEQ ID NO: 19 or SEQ ID NO:21 and a light chain variable region CDR1, CDR2, and CDR3 from a light chain variable region comprising SEQ ID NO:20 or SEQ ID NO:22.

32. An antibody that competes with the binding agent of any one of claims 1-31 for binding to human B7-H7.

33. An antibody that binds the same epitope on B7-H7 as the binding agent of any one of claims 1-31.

34. An antibody that binds an epitope on B7-H7 that overlaps with the epitope on B7-H7 bound by the binding agent of any one of claims 1-31.

35. The antibody of any one of claims 32-34, which comprises:
(a) a heavy chain variable region CDR1 comprising GYSFTDYIIV (SEQ ID NO:27), a heavy chain variable region CDR2 comprising KINPYYGTTTYNLRFED (SEQ ID NO:28), a heavy chain variable region CDR3 comprising WDYVSTLFAMDY (SEQ ID NO:29), a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31);
(b) a heavy chain variable region CDR1 comprising GYTFTEYTMH (SEQ ID NO: 13), a heavy chain variable region CDR2 comprising GINPNNGGAPYNQKFKG (SEQ ID NO:34), a heavy chain variable region CDR3 comprising GGYYFDY (SEQ ID NO: 15), a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18);
(c) a heavy chain variable region CDR1 comprising GYTFSSYSMH (SEQ ID NO:38), a heavy chain variable region CDR2 comprising TIYPGNENTSYNQKFKG (SEQ ID NO:39), a heavy chain variable region CDR3 comprising GGYYFDY (SEQ ID NO: 15), a light chain variable region CDR1 comprising KASQDVSTAVA (SEQ ID NO:35), a light chain variable region CDR2 comprising WASTRHT (SEQ ID NO:30), and a light chain variable region CDR3 comprising QQHFDIPYW (SEQ ID NO:40);
(d) a heavy chain variable region CDR1 comprising GYTFTTYTMH (SEQ ID NO:43), a heavy chain variable region CDR2 comprising YINPSRGYSDYSQKFQG (SEQ ID NO:44), a heavy chain variable region CDR3 comprising GGYDFDY (SEQ ID NO:45), a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46); or
(e) a heavy chain variable region CDR1 comprising GFSLTGYGVN (SEQ ID NO:49), a heavy chain variable region CDR2 comprising VIWGDGSTDYNSVLKS (SEQ ID NO:50), a heavy chain variable region CDR3 comprising EATEYLYWYFDV (SEQ ID NO:51), a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54).

36. The antibody of any one of claims 32-34, which comprises:
(a) a heavy chain variable region comprising SEQ ID NO:32 and a light chain variable region comprising SEQ ID NO:33;
(b) a heavy chain variable region comprising SEQ ID NO:36 and a light chain variable region comprising SEQ ID NO:37;
(c) a heavy chain variable region comprising SEQ ID NO:41 and a light chain variable region comprising SEQ ID NO:42;
(d) a heavy chain variable region comprising SEQ ID NO:47 and a light chain variable region comprising SEQ ID NO:48; or
(e) a heavy chain variable region comprising SEQ ID NO:55 and a light chain variable region comprising SEQ ID NO:56.

37. A binding agent that specifically binds human B7-H7, which comprises:
(a) a heavy chain variable region CDR1 comprising GYSFTDYIIV (SEQ ID NO:27), a heavy chain variable region CDR2 comprising KINPYYGTTTYNLRFED (SEQ ID NO:28), and a heavy chain variable region CDR3 comprising WDYVSTLFAMDY (SEQ ID NO:29); and
(b) a light chain variable region CDR1 comprising KASQDVGTAVA (SEQ ID NO: 16), a light chain variable region CDR2 comprising WASTRHT (SEQ ID NO:30), and a light chain variable region CDR3 comprising QQYKRYYT (SEQ ID NO:31).

38. The binding agent of claim 37, which comprises a heavy chain variable region having at least 80% identity to SEQ ID NO:32 and a light chain variable region having at least 80% identity to SEQ ID NO:33.

39. A binding agent that specifically binds human B7-H7, which comprises:
(a) a heavy chain variable region CDR1 comprising GYTFTEYTMH (SEQ ID NO: 13), a heavy chain variable region CDR2 comprising GINPNNGGAPYNQKFKG (SEQ ID NO:34), and a heavy chain variable region CDR3 comprising GGYYFDY (SEQ ID NO: 15); and
(b) a light chain variable region CDR1 comprising KASQDVSTAVA (SEQ ID NO:35), a light chain variable region CDR2 comprising WASTRHT (SEQ ID NO:30), and a light chain variable region CDR3 comprising QQHYDTPFT (SEQ ID NO: 18).

40. The binding agent of claim 39, which comprises a heavy chain variable region having at least 80% identity to SEQ ID NO:36 and a light chain variable region having at least 80% identity to SEQ ID NO:37.

41. A binding agent that specifically binds human B7-H7, which comprises:
(a) a heavy chain variable region CDR1 comprising GYTFSSYSMH (SEQ ID NO:38), a heavy chain variable region CDR2 comprising TIYPGNENTSYNQKFKG (SEQ ID NO:39), and a heavy chain variable region CDR3 comprising GGYYFDY (SEQ ID NO: 15); and
(b) a light chain variable region CDR1 comprising KASQDVSTAVA (SEQ ID NO:35), a light chain variable region CDR2 comprising WASTRHT (SEQ ID NO:30), and a light chain variable region CDR3 comprising (QQHFDIPYW (SEQ ID NO:40).

42. The binding agent of claim 41, which comprises a heavy chain variable region having at least 80% identity to SEQ ID NO:41 and a light chain variable region having at least 80% identity to SEQ ID NO:42.

43. A binding agent that specifically binds human B7-H7, which comprises:
(a) a heavy chain variable region CDR1 comprising GYTFTTYTMH (SEQ ID NO:43), a heavy chain variable region CDR2 comprising YINPSRGYSDYSQKFQG (SEQ ID NO:44), and a heavy chain variable region CDR3 comprising GGYDFDY (SEQ ID NO:45); and
(b) a light chain variable region CDR1 comprising KASQDVGTAVA (SEQ ID NO: 16), a light chain variable region CDR2 comprising WASTRHT (SEQ ID NO:30), and a light chain variable region CDR3 comprising QQHFITPYT (SEQ ID NO:46).

44. The binding agent of claim 43, which comprises a heavy chain variable region having at least 80% identity to SEQ ID NO:47 and a light chain variable region having at least 80% identity to SEQ ID NO:48.

45. A binding agent that specifically binds human B7-H7, which comprises:
(a) a heavy chain variable region CDR1 comprising GFSLTGYGVN (SEQ ID NO:49), a heavy chain variable region CDR2 comprising VIWGDGSTDYNSVLKS (SEQ ID NO:50), and a heavy chain variable region CDR3 comprising EATEYLYWYFDV (SEQ ID NO:51); and
(b) a light chain variable region CDR1 comprising RASESVEYYGSSLMQ (SEQ ID NO:52), a light chain variable region CDR2 comprising AASNVES (SEQ ID NO:53), and a light chain variable region CDR3 comprising QQGRRVPWT (SEQ ID NO:54).

46. The binding agent of claim 45, which comprises a heavy chain variable region having at least 80% identity to SEQ ID NO:55 and a light chain variable region having at least 80% identity to SEQ ID NO:56.

47. A binding agent that specifically binds human B7-H7, which comprises:
(a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SVYDAPWLAH (SEQ ID NO:59); and
(b) a light chain variable region CDR1 comprising RASENIYIYLA (SEQ ID NO:60), a light chain variable region CDR2 comprising NAKTLAE (SEQ ID NO:61), and a light chain variable region CDR3 comprising QHHYGTPPT (SEQ ID NO:62).

48. The binding agent of claim 47, which comprises a heavy chain variable region having at least 80% identity to SEQ ID NO:63 and a light chain variable region having at least 80% identity to SEQ ID NO:64.

49. A binding agent that specifically binds human B7-H7, which comprises:
(a) a heavy chain variable region CDR1 comprising GYSFTGYNMN (SEQ ID NO:57), a heavy chain variable region CDR2 comprising NIDPYSGGSTYNQKFKG (SEQ ID NO:58), and a heavy chain variable region CDR3 comprising SFYDAPYLTY (SEQ ID NO:65); and
(b) a light chain variable region CDR1 comprising RASENIFIYLA (SEQ ID NO:66), a light chain variable region CDR2 comprising NAKTLAE (SEQ ID NO:61), and a light chain variable region CDR3 comprising QHHYGTPPT (SEQ ID NO:62).

50. The binding agent of claim 49, which comprises a heavy chain variable region having at least 80% identity to SEQ ID NO:67 and a light chain variable region having at least 80% identity to SEQ ID NO:68.

51. A binding agent that specifically binds human B7-H7, which comprises:
(a) a heavy chain variable region CDR1 comprising GYTFTSFWIH (SEQ ID NO:69), a heavy chain variable region CDR2 comprising YIIPNTDYTEYNQKFKD (SEQ ID NO:70), and a heavy chain variable region CDR3 comprising GLRGAYYFDY (SEQ ID NO:71); and
(b) a light chain variable region CDR1 comprising RSSQSVSTSTNGYMH (SEQ ID NO:72), a light chain variable region CDR2 comprising YASNLES (SEQ ID NO:73), and a light chain variable region CDR3 comprising QHSWVLPYT (SEQ ID NO:74).

52. The binding agent of claim 51, which comprises a heavy chain variable region having at least 80% identity to SEQ ID NO:75 and a light chain variable region having at least 80% identity to SEQ ID NO:76.

53. The binding agent of any one of claims 37-52, which is an antibody.

54. The binding agent of any one of claims 37-52, which is a monoclonal antibody.

55. The binding agent of any one of claims 37-54, which is a chimeric antibody or a humanized antibody.

56. The binding agent of any one of claims 37-55, which is a bispecific antibody or a multispecific antibody.

57. The binding agent of any one of claims 37-54, which is an antibody fragment comprising at least one antigen-binding site.

58. The binding agent of any one of claims 37-54, which is a Fab, Fab′, F(ab′)₂, Fv, scFv, (scFv)₂, single chain antibody, dual variable region antibody, or single variable region antibody.

59. The binding agent of any one of claims 37-56, which is an IgG1 antibody.

60. The binding agent of any one of claims 37-56, which is an IgG2 antibody.

61. The binding agent of any one of claims 37-56, which is an IgG4 antibody.

62. The binding agent of any one of claims 37-61, which comprises a kappa light chain.

63. The binding agent of any one of claims 37-61, which comprises a lambda light chain.

64. The binding agent of any one of claims 1-63, which binds the extracellular domain of B7-H7.

65. The binding agent of any one of claims 1-63, which binds within the IgV-type domain 1 of human B7-H7.

66. The binding agent of any one of claims 1-63, which binds within an Ig-like domain comprising amino acids 61-131 of SEQ ID NOT.

67. The binding agent of claim 51, which binds within the IgC-type domain of human B7-H7.

68. The binding agent of claim 51, which binds within an Ig-like domain comprising amino acids 138-222 of SEQ ID NOT.

69. The binding agent of any one of claims 1-68, which inhibits binding of B7-H7 to a B7-H7 receptor.

70. The binding agent of any one of claims 1-68, which inhibits or blocks the interaction between B7-H7 and a B7-H7 receptor.

71. The binding agent of claim 69 or claim 70, wherein the B7-H7 receptor is KIR3DL3.

72. The binding agent of claim 69 or claim 70, wherein the B7-H7 receptor is CD28H.

73. The binding agent of any one of claims 1-72, which induces and/or increases an immune response.

74. The binding agent of claim 73, wherein the immune response is directed to a tumor or tumor cell.

75. The binding agent of any one of claims 1-72, which increases cell-mediated immunity.

76. The binding agent of any one of claims 1-72, which increases natural killer (NK) cell activity.

77. The binding agent of any one of claims 1-72, which inhibits the suppression of NK activity.

78. The binding agent of claim 76 or claim 77, wherein the NK cell activity is directed to a tumor or tumor cells.

79. A pharmaceutical composition that comprises the binding agent of any one of claims 1-68 and a pharmaceutically acceptable carrier.

80. An isolated polynucleotide molecule comprising a nucleotide sequence that encodes the binding agent of any one of claims 1-68.

81. A vector comprising the polynucleotide of claim 80.

82. An isolated cell comprising the polynucleotide of claim 80.

83. An isolated cell comprising the vector of claim 81.

84. An isolated cell producing the binding agent of any one of claims 1-68.

85. A hybridoma that produces the monoclonal antibody of any one of claims 16, 29, or 54.

86. A method of inducing, activating, promoting, increasing, enhancing, or prolonging an immune response in a subject, comprising administering to the subject a therapeutically effective amount of the binding agent of any one of claims 1-68.

87. The method of claim 86, wherein the immune response is against a tumor or cancer.

88. A method of increasing NK cell activity in a subject, wherein the method comprises administering to the subject a therapeutically effective amount of a binding agent of any one of claims 1-68.

89. A method of inhibiting the suppression of NK cell activity in a subject, wherein the method comprises administering to the subject a therapeutically effective amount of a binding agent of any one of claims 1-68.

90. A method of inhibiting growth of tumor cells, wherein the method comprises contacting the tumor cells with an effective amount of a binding agent of any one of claims 1-68.

91. A method of inhibiting growth of a tumor in a subject, wherein the method comprises administering to the subject a therapeutically effective amount of a binding agent of any one of claims 1-68.

92. A method of treating cancer in a subject, wherein the method comprises administering to the subject a therapeutically effective amount of a binding agent of any one of claims 1-68.

93. The method of any one of claims 86-92, which further comprises administering at least one additional therapeutic agent.

94. The method of claim 93, wherein the additional therapeutic agent is a chemotherapeutic agent.

95. The method of claim 93, wherein the additional therapeutic agent is an immunomodulatory agent.

96. The method of claim 93, wherein the additional therapeutic agent is an immune checkpoint inhibitor.

97. The method of claim 96, wherein the immune checkpoint inhibitor is selected from the group consisting of an anti-PD-1 antibody, an anti-PD-L1 antibody, an anti-CTLA4 antibody, an anti-LAG3 antibody, an anti-TIM3 antibody, an anti-GITR antibody, an anti-OX40 antibody, an anti-4-1-BB antibody, and an anti-CD28 antibody.

98. The method of any one of claims 86-89 and 91-97, wherein the subject is a human.

99. The method of claim 98, wherein the binding agent is administered intravenously.


SEQUENCE LISTING (161 sequences)

SEQ ID NO: 1 - protein, 414 aa, Homo sapiens
MKAQTALSFFLILITSLSGSQGIFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 2 - protein, 392 aa, Homo sapiens
IFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 3 - protein, 323 aa, Homo sapiens
IFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNK

SEQ ID NO: 4 - protein, 71 aa, Homo sapiens
IHWKYQDSYKVHSYYKGSDHLESQDPRYANRTSLFYNEIQNGNASLFFRRVSLLDEGIYTCYVGTAIQVIT

SEQ ID NO: 5 - protein, 85 aa, Homo sapiens
VGVFLTPVMKYEKRNTNSFLICSVLSVYPRPIITWKMDNTPISENNMEETGSLDSFSINSPLNITGSNSSYECTIENSLLKQTWT

SEQ ID NO: 6 - protein, 94 aa, Homo sapiens
QSEHVSLSCQPVNDYFSPNQDFKVTWSRMKSGTFSVLAYYLSSSQNTIINESRFSWNKELINQSDFSMNLMDLNLSDSGEYLCNISSDEYTLLT

SEQ ID NO: 7 - protein, 411 aa, Macaca fascicularis
MKAQTSFFLILISSLSGSQGIFLSAFFTYVPMNEQIIIGRLGEDIILPSSFERGSEVVIHWKYQDSYNSYNVHSYYKGSGRLESQDTRYANRTSLFYNEIQNGNASLFFRRLSLLDEGIYTCYVGTAIQAITNKVVLKVGVFLTPMMKYEKRNTNSFLICNVLSVYPRPIITWKMDNTPISENNMQETGSLGPFSINSTLNITGSNSSYECTIENSLLKQTWTGRWTMKDGLHKMQSEHVSLSCELVNDYFSPNQDFKVTWSRMESGISSILAYYLSSSQNTTFYESRFSWNKELKNQSDFSMNLTDLSLSDSGEYLCNISSDEYTLLTIHTVHVEPSQETASDNKVLWILVASLILVLCLIWLIWKVKCCTERRRVSTESTHDDNASDNAEENMRLSGRVQQMREDLDNS

SEQ ID NO: 8 - protein, 391 aa, Macaca fascicularis
IFLSAFFTYVPMNEQIIIGRLGEDIILPSSFERGSEVVIHWKYQDSYNSYNVHSYYKGSGRLESQDTRYANRTSLFYNEIQNGNASLFFRRLSLLDEGIYTCYVGTAIQAITNKVVLKVGVFLTPMMKYEKRNTNSFLICNVLSVYPRPIITWKMDNTPISENNMQETGSLGPFSINSTLNITGSNSSYECTIENSLLKQTWTGRWTMKDGLHKMQSEHVSLSCELVNDYFSPNQDFKVTWSRMESGISSILAYYLSSSQNTTFYESRFSWNKELKNQSDFSMNLTDLSLSDSGEYLCNISSDEYTLLTIHTVHVEPSQETASDNKVLWILVASLILVLCLIWLIWKVKCCTERRRVSTESTHDDNASDNAEENMRLSGRVQQMREDLDNS

SEQ ID NO: 9 - protein, 326 aa, Macaca fascicularis
IFLSAFFTYVPMNEQIIIGRLGEDIILPSSFERGSEVVIHWKYQDSYNSYNVHSYYKGSGRLESQDTRYANRTSLFYNEIQNGNASLFFRRLSLLDEGIYTCYVGTAIQAITNKVVLKVGVFLTPMMKYEKRNTNSFLICNVLSVYPRPIITWKMDNTPISENNMQETGSLGPFSINSTLNITGSNSSYECTIENSLLKQTWTGRWTMKDGLHKMQSEHVSLSCELVNDYFSPNQDFKVTWSRMESGISSILAYYLSSSQNTTFYESRFSWNKELKNQSDFSMNLTDLSLSDSGEYLCNISSDEYTLLTIHTVHVEPSQETASDNK

SEQ ID NO: 10 - protein, 74 aa, Macaca fascicularis
IHWKYQDSYNSYNVHSYYKGSGRLESQDTRYANRTSLFYNEIQNGNASLFFRRLSLLDEGIYTCYVGTAIQAIT

SEQ ID NO: 11 - protein, 85 aa, Macaca fascicularis
VGVFLTPMMKYEKRNTNSFLICNVLSVYPRPIITWKMDNTPISENNMQETGSLGPFSINSTLNITGSNSSYECTIENSLLKQTWT

SEQ ID NO: 12 - protein, 94 aa, Macaca fascicularis
QSEHVSLSCELVNDYFSPNQDFKVTWSRMESGISSILAYYLSSSQNTTFYESRFSWNKELKNQSDFSMNLTDLSLSDSGEYLCNISSDEYTLLT

SEQ ID NO: 13 - protein, 10 aa, Artificial Sequence
  source: /note=“Description of Artificial Sequence Synthetic peptide”
GYTFTEYTMH

SEQ ID NO: 14 - protein, 17 aa, Artificial Sequence
  source: /note=“Description of Artificial Sequence Synthetic peptide”
GINPNNYGAPYNQKFKG

SEQ ID NO: 15 - protein, 7 aa, Artificial Sequence
  source: /note=“Description of Artificial Sequence Synthetic peptide”
GGYYFDY

SEQ ID NO: 16 - protein, 11 aa, Artificial Sequence
  source: /note=“Description of Artificial Sequence Synthetic peptide”
KASQDVGTAVA

SEQ ID NO: 17 - protein, 7 aa, Artificial Sequence
  source: /note=“Description of Artificial Sequence Synthetic peptide”
WAFTRHT

SEQ ID NO: 18 - protein, 9 aa, Artificial Sequence
  source: /note=“Description of Artificial Sequence Synthetic peptide”
QQHYDTPFT

SEQ ID NO: 19 - protein, 116 aa, Artificial Sequence
  source: /note=“Description of Artificial Sequence Synthetic polypeptide”
EVQLQQSGPELVKPGASVKISCKTSGYTFTEYTMHWVKQSHGKSLEWIGGINPNNYGAPYNQKFKGKATLTVDKSSNTAYMELRSLTSEDSAIYYCASGGYYFDYWGQGTTLTVSS

SEQ ID NO: 20 - protein, 107 aa, Artificial Sequence
  source: /note=“Description of Artificial Sequence Synthetic polypeptide”
DIVMTQSHKFMSTSFGDRVSITCKASQDVGTAVAWYQQKLGQSPKLLISWAFTRHTGVPDRFTGSGSGTDYTLTISSLQAEDLALYYCQQHYDTPFTFGSGTKLEIK

SEQ ID NO: 21 - protein, 116 aa, Artificial Sequence
  source: /note=“Description of Artificial Sequence Synthetic polypeptide”
QVQLVQSGAEVKKPGASVKVSCKTSGYTFTEYTMHWVRQAPGQGLEWMGGINPNNYGAPYNQKFKGRVTMTVDTSISTAYMELSRLRSDDTAVYYCASGGYYFDYWGQGTLVTVSS

SEQ ID NO: 22 - protein, 107 aa, Artificial Sequence
  source: /note=“Description of Artificial Sequence Synthetic polypeptide”
DIQMTQSPSSLSASVGDRVTITCKASQDVGTAVAWYQQKPGKAPKLLIYWAFTRHTGVPSRFSGSGSGTDYTLTISSLQPEDFATYYCQQHYDTPFTFGQGTKVEIK

SEQ ID NO: 23 - protein, 468 aa, Artificial Sequence
  source: /note=“Description of Artificial Sequence Synthetic polypeptide”
MDMRVPAQLLGLLLLWLRGARCQVQLVQSGAEVKKPGASVKVSCKTSGYTFTEYTMHWVRQAPGQGLEWMGGINPNNYGAPYNQKFKGRVTMTVDTSISTAYMELSRLRSDDTAVYYCASGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 446 aa, Artificial Sequence
  source: /note=“Description of Artificial Sequence Synthetic polypeptide”
QVQLVQSGAEVKKPGASVKVSCKTSGYTFTEYTMHWVRQAPGQGLEWMGGINPNNYGAPYNQKFKGRVTMTVDTSISTAYMELSRLRSDDTAVYYCASGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 236 aa, Artificial Sequence
  source: /note=“Description of Artificial Sequence Synthetic polypeptide”
MDMRVPAQLLGLLLLWLRGARCDIQMTQSPSSLSASVGDRVTITCKASQDVGTAVAWYQQKPGKAPKLLIYWAFTRHTGVPSRFSGSGSGTDYTLTISSLQPEDFATYYCQQHYDTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, Artificial Sequence
  source: /note=“Description of Artificial Sequence Synthetic polypeptide”
DIQMTQSPSSLSASVGDRVTITCKASQDVGTAVAWYQQKPGKAPKLLIYWAFTRHTGVPSRFSGSGSGTDYTLTISSLQPEDFATYYCQQHYDTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 10 aa, Artificial Sequence
  source: /note=“Description of Artificial Sequence Synthetic peptide”
GYSFTDYIIV

SEQ ID NO: 28 - protein, 17 aa, Artificial Sequence
  source: /note=“Description of Artificial Sequence Synthetic peptide”
KINPYYGTTTYNLRFED

SEQ ID NO: 29 - protein, 12 aa, Artificial Sequence
  source: /note=“Description of Artificial Sequence Synthetic peptide”
WDYVSTLFAMDY

SEQ ID NO: 30 - protein, 7 aa, Artificial Sequence
  source: /note=“Description of Artificial Sequence Synthetic peptide”
WASTRHT

SEQ ID NO: 31 - protein, 8 aa, Artificial Sequence
  source: /note=“Description of Artificial Sequence Synthetic peptide”
QQYKRYYT

SEQ ID NO: 32 - protein, 121 aa, Artificial Sequence
  source: /note=“Description of Artificial Sequence Synthetic polypeptide”
EIQLQQTGPELVKPGASLRISCKASGYSFTDYIIVWVKQNQGKNLDWIEKINPYYGTTTYNLRFEDKATLTVNKSSTTAYMQLNSLTSEDSAVYYCARWDYVSTLFAMDYWGQGTSITVSS

SEQ ID NO: 33 - protein, 106 aa, Artificial Sequence
  source: /note=“Description of Artificial Sequence Synthetic polypeptide”
DIVMTQSHKVMSTSVGDRVSIICKASQDVGTAVAWYQQKPGQSPKLLIYWASTRHTGVPDRFTGSGSGTDFILTITNVQSENLANYFCQQYKRYYTFGGGTKLEIK

SEQ ID NO: 34 - protein, 17 aa, Artificial Sequence
  source: /note=“Description of Artificial Sequence Synthetic peptide”
GINPNNGGAPYNQKFKG

SEQ ID NO: 35 - protein, 11 aa, Artificial Sequence
  source: /note=“Description of Artificial Sequence Synthetic peptide”
KASQDVSTAVA

SEQ ID NO: 36 - protein, 116 aa, Artificial Sequence
  source: /note=“Description of Artificial Sequence Synthetic polypeptide”
EVQLQQSGPELVKPGASVKISCKTSGYTFTEYTMHWVKQSHGKSLEWIGGINPNNGGAPYNQKFKGKATLTVDKSSSTAYMELRSLTSEDSAVYYCARGGYYFDYWGQGTTLTVSS

SEQ ID NO: 37 - protein, 107 aa, Artificial Sequence
  source: /note=“Description of Artificial Sequence Synthetic polypeptide”
DIVMTQSHKFMSTSVGDRVSITCKASQDVSTAVAWYQQKSGQSPKLLIYWASTRHTGVPDRFTGSGSGTDYTLTISSVQAEDLALYYCQQHYDTPFTFGGGTKLEIK

SEQ ID NO: 38 - protein, 10 aa, Artificial Sequence
  source: /note=“Description of Artificial Sequence Synthetic peptide”
GYTFSSYSMH

SEQ ID NO: 39 - protein, 17 aa, Artificial Sequence
  source: /note=“Description of Artificial Sequence Synthetic peptide”
TIYPGNENTSYNQKFKG

SEQ ID NO: 40 - protein, 9 aa, Artificial Sequence
  source: /note=“Description of Artificial Sequence Synthetic peptide”
QQHFDIPYW

SEQ ID NO: 41 - protein, 116 aa, Artificial Sequence
  source: /note=“Description of Artificial Sequence Synthetic polypeptide”
QEQLQQPGTELVKPGASVKISCKASGYTFSSYSMHWVKLTPGQGLEWMGTIYPGNENTSYNQKFKGKATLTADKSSSTAYMQLSSLTSEDSAVYYCARGGYYFDYWGQGTTLTVSS

SEQ ID NO: 42 - protein, 107 aa, Artificial Sequence
  source: /note=“Description of Artificial Sequence Synthetic polypeptide”
DIVMTQSHKFLSTSVGDRVSITCKASQDVSTAVAWYQQKPGQSPKLLIYWASTRHTGVSDRFTGSGSGTDFTLTISSVQAEDLALYYCQQHFDIPYWFGGGTKLEIK

SEQ ID NO: 43 - protein, 10 aa, Artificial Sequence
  source: /note=“Description of Artificial Sequence Synthetic peptide”
GYTFTTYTMH

SEQ ID NO: 44 - protein, 17 aa, Artificial Sequence
  source: /note=“Description of Artificial Sequence Synthetic peptide”
YINPSRGYSDYSQKFQG

SEQ ID NO: 45 - protein, 7 aa, Artificial Sequence
  source: /note=“Description of Artificial Sequence Synthetic peptide”
GGYDFDY

SEQ ID NO: 46 - protein, 9 aa, Artificial Sequence
  source: /note=“Description of Artificial Sequence Synthetic peptide”
QQHFITPYT

SEQ ID NO: 47 - protein, 116 aa, Artificial Sequence
  source: /note=“Description of Artificial Sequence Synthetic polypeptide”
QVQLQQSAAELARPGASVKMSCKASGYTFTTYTMHWVKQRPGQGLEWIGYINPSRGYSDYSQKFQGKSTLTTDKSSNTAYIQLTSLTSEDSAVYFCARGGYDFDYWGQGTTLTVSS

SEQ ID NO: 48 - protein, 107 aa, Artificial Sequence
  source: /note=“Description of Artificial Sequence Synthetic polypeptide”
DIVMTQSHKFMSTSVGDRVSITCKASQDVGTAVAWYQQKPGQSPKLLLYWASTRHTGVPDRFTGSGSGTDYTLTISSVQAEDLALYHCQQHFITPYTFGGGTKLEIK

SEQ ID NO: 49 - protein, 10 aa, Artificial Sequence
  source: /note=“Description of Artificial Sequence Synthetic peptide”
GFSLTGYGVN

SEQ ID NO: 50 - protein, 16 aa, Artificial Sequence
  source: /note=“Description of Artificial Sequence Synthetic peptide”
VIWGDGSTDYNSVLKS

SEQ ID NO: 51 - protein, 12 aa, Artificial Sequence
  source: /note=“Description of Artificial Sequence Synthetic peptide”
EATEYLYWYFDV

SEQ ID NO: 52 - protein, 15 aa, Artificial Sequence
  source: /note=“Description of Artificial Sequence Synthetic peptide”
RASESVEYYGSSLMQ

SEQ ID NO: 53 - protein, 7 aa, Artificial Sequence
  source: /note=“Description of Artificial Sequence Synthetic peptide”
AASNVES

SEQ ID NO: 54 - protein, 9 aa, Artificial Sequence
  source: /note=“Description of Artificial Sequence Synthetic peptide”
QQGRRVPWT

SEQ ID NO: 55 - protein, 120 aa, Artificial Sequence
  source: /note=“Description of Artificial Sequence Synthetic polypeptide”
QVQLKESGPGLVAPSQSLSITCTVSGFSLTGYGVNWVRQPPGKGLEWLGVIWGDGSTDYNSVLKSRLSISKDNSKSQVFLKMNSLQTDDTARYYCAREATEYLYWYFDVWGAGTTVTVSS

SEQ ID NO: 56 - protein, 111 aa, Artificial Sequence
  source: /note=“Description of Artificial Sequence Synthetic polypeptide”
DIVLTQSPASLAVSLGQRATISCRASESVEYYGSSLMQWYQQKPGQPPKLLIFAASNVESGVPPRFSGSGSGTDFNLNIHPVDEDDIAMYFCQQGRRVPWTFGGGTKLEIK

SEQ ID NO: 57 - protein, 10 aa, Artificial Sequence
  source: /note=“Description of Artificial Sequence Synthetic peptide”
GYSFTGYNMN

SEQ ID NO: 58 - protein, 17 aa, Artificial Sequence
  source: /note=“Description of Artificial Sequence Synthetic peptide”
NIDPYSGGSTYNQKFKG

SEQ ID NO: 59 - protein, 10 aa, Artificial Sequence
  source: /note=“Description of Artificial Sequence Synthetic peptide”
SVYDAPWLAH

SEQ ID NO: 60 - protein, 11 aa, Artificial Sequence
  source: /note=“Description of Artificial Sequence Synthetic peptide”
RASENIYIYLA

SEQ ID NO: 61 - protein, 7 aa, Artificial Sequence
  source: /note=“Description of Artificial Sequence Synthetic peptide”
NAKTLAE

SEQ ID NO: 62 - protein, 9 aa, Artificial Sequence
  source: /note=“Description of Artificial Sequence Synthetic peptide”
QHHYGTPPT

SEQ ID NO: 63 - protein, 119 aa, Artificial Sequence
  source: /note=“Description of Artificial Sequence Synthetic polypeptide”
EVQLQQSGPELEKPGASVKISCKASGYSFTGYNMNWVKESNGRSLEWIGNIDPYSGGSTYNQKFKGKATMTVDKSSSTAYMQLKSLTSEDSAVYYCARSVYDAPWLAHWGQGTLVTVSA

SEQ ID NO: 64 - protein, 107 aa, Artificial Sequence
  source: /note=“Description of Artificial Sequence Synthetic polypeptide”
DIQMTQSPASLSASVGETVTITCRASENIYIYLAWYQQKQGKSPQLLVYNAKTLAEGVPSRFSGSGSGTQFSLKINRLQPEDFGNYYCQHHYGTPPTFGGGTKLEIK

SEQ ID NO: 65 - protein, 10 aa, Artificial Sequence
  source: /note=“Description of Artificial Sequence Synthetic peptide”
SFYDAPYLTY

SEQ ID NO: 66 - protein, 11 aa, Artificial Sequence
  source: /note=“Description of Artificial Sequence Synthetic peptide”
RASENIFIYLA

SEQ ID NO: 67 - protein, 119 aa, Artificial Sequence
  source: /note=“Description of Artificial Sequence Synthetic polypeptide”
EVQLQQSGPELEKPGASVKISCKASGYSFTGYNMNWVKQSNGKSLEWIGNIDPYSGGSTYNQKFKGKATLTVDKSSSTASLQLKSLTSEDSSVYYCARSFYDAPYLTYWGQGTLVTVSA

SEQ ID NO: 68 - protein, 106 aa, Artificial Sequence
  source: /note=“Description of Artificial Sequence Synthetic polypeptide”
DIQMTQSPTSLSASVGETVTITCRASENIFIYLAWYQQKQGKSPQLLVFNAKTLAEGVPSRFSGSGSGTQFSLKINSLRPEDFGTYYCQHHYGTPPTFGGGTKLEV

SEQ ID NO: 69 - protein, 10 aa, Artificial Sequence
  source: /note=“Description of Artificial Sequence Synthetic peptide”
GYTFTSFWIH

SEQ ID NO: 70 - protein, 17 aa, Artificial Sequence
  source: /note=“Description of Artificial Sequence Synthetic peptide”
YIIPNTDYTEYNQKFKD

SEQ ID NO: 71 - protein, 10 aa, Artificial Sequence
  source: /note=“Description of Artificial Sequence Synthetic peptide”
GLRGAYYFDY

SEQ ID NO: 72 - protein, 15 aa, Artificial Sequence
  source: /note=“Description of Artificial Sequence Synthetic peptide”
RSSQSVSTSTNGYMH

SEQ ID NO: 73 - protein, 7 aa, Artificial Sequence
  source: /note=“Description of Artificial Sequence Synthetic peptide”
YASNLES

SEQ ID NO: 74 - protein, 9 aa, Artificial Sequence
  source: /note=“Description of Artificial Sequence Synthetic peptide”
QHSWVLPYT

SEQ ID NO: 75 - protein, 119 aa, Artificial Sequence
  source: /note=“Description of Artificial Sequence Synthetic polypeptide”
QVQLQQSGAELAKPGASVKMSCKASGYTFTSFWIHWIKQRPGQGLEWIGYIIPNTDYTEYNQKFKDKATFTADKSSSTAYMQLSSLTSEDSAVFYCARGLRGAYYFDYWGQGTTLTVSS

SEQ ID NO: 76 - protein, 111 aa, Artificial Sequence
  source: /note=“Description of Artificial Sequence Synthetic polypeptide”
DIVLTQSPASLAVSLGLRATISCRSSQSVSTSTNGYMHWYQQKPGQPPKLLIMYASNLESGVPARFSGSGSGTDFTLNIHPVDEEDTATYYCQHSWVLPYTFGGGTKLEIK

SEQ ID NO: 77 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 282 aa, Homo sapiens
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 82 - protein, 128 aa, Homo sapiens
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPG

SEQ ID NO: 83 - protein, 410 aa, Homo sapiens
MSLMVVSMACVGFFLLEGPWPHVGGQDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTHLPYELSAPSDPLDIVVVGLYGKPSLSAQPGPTVQAGENVTLSCSSRSLFDIYHLSREAEAGELRLTAVLRVNGTFQANFPLGPVTHGGNYRCFGSFRALPHAWSDPSDPLPVSVTGNSRHLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 84 - protein, 297 aa, Homo sapiens
QDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTHLPYELSAPSDPLDIVVVGLYGKPSLSAQPGPTVQAGENVTLSCSSRSLFDIYHLSREAEAGELRLTAVLRVNGTFQANFPLGPVTHGGNYRCFGSFRALPHAWSDPSDPLPVSVTGNSRHL

SEQ ID NO: 85 - protein, 7 aa, Artificial Sequence
  source: /note=“Description of Artificial Sequence Synthetic peptide”
GYTFTEY

SEQ ID NO: 86 - protein, 6 aa, Artificial Sequence
  source: /note=“Description of Artificial Sequence Synthetic peptide”
NPNNYG

SEQ ID NO: 87 - protein, 10 aa, Artificial Sequence
  source: /note=“Description of Artificial Sequence Synthetic peptide”
GINPNNYGAP

SEQ ID NO: 88 - protein, 5 aa, Artificial Sequence
  source: /note=“Description of Artificial Sequence Synthetic peptide”
EYTMH

SEQ ID NO: 89 - protein, 6 aa, Artificial Sequence
  source: /note=“Description of Artificial Sequence Synthetic peptide”
TEYTMH

SEQ ID NO: 90 - protein, 13 aa, Artificial Sequence
  source: /note=“Description of Artificial Sequence Synthetic peptide”
WIGGINPNNYGAP

SEQ ID NO: 91 - protein, 8 aa, Artificial Sequence
  source: /note=“Description of Artificial Sequence Synthetic peptide”
ASGGYYFD

SEQ ID NO: 92 - protein, 7 aa, Artificial Sequence
  source: /note=“Description of Artificial Sequence Synthetic peptide”
GTAVAWY

SEQ ID NO: 93 - protein, 10 aa, Artificial Sequence
  source: /note=“Description of Artificial Sequence Synthetic peptide”
LLISWAFTRH

SEQ ID NO: 94 - protein, 8 aa, Artificial Sequence
  source: /note=“Description of Artificial Sequence Synthetic peptide”
QQHYDTPF

SEQ ID NO: 95 - protein, 13 aa, Artificial Sequence
  source: /note=“Description of Artificial Sequence Synthetic peptide”
WMGGINPNNYGAP

SEQ ID NO: 96 - protein, 10 aa, Artificial Sequence
  source: /note=“Description of Artificial Sequence Synthetic peptide”
LLIYWAFTRH

SEQ ID NO: 97 - protein, 7 aa, Artificial Sequence
  source: /note=“Description of Artificial Sequence Synthetic peptide”
GYSFTDY

SEQ ID NO: 98 - protein, 6 aa, Artificial Sequence
  source: /note=“Description of Artificial Sequence Synthetic peptide”
NPYYGT

SEQ ID NO: 99 - protein, 10 aa, Artificial Sequence
  source: /note=“Description of Artificial Sequence Synthetic peptide”
KINPYYGTTT

SEQ ID NO: 100 - protein, 5 aa, Artificial Sequence
  source: /note=“Description of Artificial Sequence Synthetic peptide”
DYIIV

SEQ ID NO: 101 - protein, 6 aa, Artificial Sequence
  source: /note=“Description of Artificial Sequence Synthetic peptide”
TDYIIV

SEQ ID NO: 102 - protein, 13 aa, Artificial Sequence
  source: /note=“Description of Artificial Sequence Synthetic peptide”
WIEKINPYYGTTT

SEQ ID NO: 103 - protein, 13 aa, Artificial Sequence
  source: /note=“Description of Artificial Sequence Synthetic peptide”
ARWDYVSTLFAMD

SEQ ID NO: 104 - protein, 10 aa, Artificial Sequence
  source: /note=“Description of Artificial Sequence Synthetic peptide”
LLIYWASTRH

SEQ ID NO: 105 - protein, 7 aa, Artificial Sequence
  source: /note=“Description of Artificial Sequence Synthetic peptide”
QQYKRYY

SEQ ID NO: 106 - protein, 6 aa, Artificial Sequence
  source: /note=“Description of Artificial Sequence Synthetic peptide”
NPNNGG

SEQ ID NO: 107 - protein, 10 aa, Artificial Sequence
  source: /note=“Description of Artificial Sequence Synthetic peptide”
GINPNNGGAP

SEQ ID NO: 108 - protein, 13 aa, Artificial Sequence
  source: /note=“Description of Artificial Sequence Synthetic peptide”
WIGGINPNNGGAP

SEQ ID NO: 109 - protein, 8 aa, Artificial Sequence
  source: /note=“Description of Artificial Sequence Synthetic peptide”
ARGGYYFD

SEQ ID NO: 110 - protein, 7 aa, Artificial Sequence
  source: /note=“Description of Artificial Sequence Synthetic peptide”
STAVAWY

SEQ ID NO: 111 - protein, 10 aa, Artificial Sequence
  source: /note=“Description of Artificial Sequence Synthetic peptide”
LLIYWASTRH

SEQ ID NO: 112 - protein, 7 aa, Artificial Sequence
  source: /note=“Description of Artificial Sequence Synthetic peptide”
GYTFSSY

SEQ ID NO: 113 - protein, 6 aa, Artificial Sequence
  source: /note=“Description of Artificial Sequence Synthetic peptide”
YPGNEN

SEQ ID NO: 114 - protein, 10 aa, Artificial Sequence
  source: /note=“Description of Artificial Sequence Synthetic peptide”
TIYPGNENTS

SEQ ID NO: 115 - protein, 5 aa, Artificial Sequence
  source: /note=“Description of Artificial Sequence Synthetic peptide”
SYSMH

SEQ ID NO: 116 - protein, 6 aa, Artificial Sequence
  source: /note=“Description of Artificial Sequence Synthetic peptide”
SSYSMH

SEQ ID NO: 117 - protein, 13 aa, Artificial Sequence
  source: /note=“Description of Artificial Sequence Synthetic peptide”
WMGTIYPGNENTS

SEQ ID NO: 118 - protein, 8 aa, Artificial Sequence
  source: /note=“Description of Artificial Sequence Synthetic peptide”
QQHFDIPY

SEQ ID NO: 119 - protein, 7 aa, Artificial Sequence
  source: /note=“Description of Artificial Sequence Synthetic peptide”
GYTFTTY

SEQ ID NO: 120 - protein, 6 aa, Artificial Sequence
  source: /note=“Description of Artificial Sequence Synthetic peptide”
NPSRGY

SEQ ID NO: 121 - protein, 10 aa, Artificial Sequence
  source: /note=“Description of Artificial Sequence Synthetic peptide”
YINPSRGYSD

SEQ ID NO: 122 - protein, 5 aa, Artificial Sequence
  source: /note=“Description of Artificial Sequence Synthetic peptide”
TYTMH

SEQ ID NO: 123 - protein, 6 aa, Artificial Sequence
  source: /note=“Description of Artificial Sequence Synthetic peptide”
TTYTMH

SEQ ID NO: 124 - protein, 13 aa, Artificial Sequence
  source: /note=“Description of Artificial Sequence Synthetic peptide”
WIGYINPSRGYSD

SEQ ID NO: 125 - protein, 8 aa, Artificial Sequence
  source: /note=“Description of Artificial Sequence Synthetic peptide”
ARGGYDFD

SEQ ID NO: 126 - protein, 10 aa, Artificial Sequence
  source: /note=“Description of Artificial Sequence Synthetic peptide”
LLLYWASTRH

SEQ ID NO: 127 - protein, 8 aa, Artificial Sequence
  source: /note=“Description of Artificial Sequence Synthetic peptide”
QQHFITPY

SEQ ID NO: 128 - protein, 7 aa, Artificial Sequence
  source: /note=“Description of Artificial Sequence Synthetic peptide”
GFSLTGY

SEQ ID NO: 129 - protein, 5 aa, Artificial Sequence
  source: /note=“Description of Artificial Sequence Synthetic peptide”
WGDGS

SEQ ID NO: 130 - protein, 9 aa, Artificial Sequence
  source: /note=“Description of Artificial Sequence Synthetic peptide”
VIWGDGSTD

SEQ ID NO: 131 - protein, 5 aa, Artificial Sequence
  source: /note=“Description of Artificial Sequence Synthetic peptide”
GYGVN

SEQ ID NO: 132 - protein, 6 aa, Artificial Sequence
  source: /note=“Description of Artificial Sequence Synthetic peptide”
TGYGVN

SEQ ID NO: 133 - protein, 12 aa, Artificial Sequence
  source: /note=“Description of Artificial Sequence Synthetic peptide”
WLGVIWGDGSTD

SEQ ID NO: 134 - protein, 13 aa, Artificial Sequence
  source: /note=“Description of Artificial Sequence Synthetic peptide”
AREATEYLYWYFD

SEQ ID NO: 135 - protein, 11 aa, Artificial Sequence
  source: /note=“Description of Artificial Sequence Synthetic peptide”
EYYGSSLMQWY

SEQ ID NO: 136 - protein, 10 aa, Artificial Sequence
  source: /note=“Description of Artificial Sequence Synthetic peptide”
LLIFAASNVE

SEQ ID NO: 137 - protein, 8 aa, Artificial Sequence
  source: /note=“Description of Artificial Sequence Synthetic peptide”
QQGRRVPW

SEQ ID NO: 138 - protein, 7 aa, Artificial Sequence
  source: /note=“Description of Artificial Sequence Synthetic peptide”
GYSFTGY

SEQ ID NO: 139 - protein, 6 aa, Artificial Sequence
  source: /note=“Description of Artificial Sequence Synthetic peptide”
DPYSGG

SEQ ID NO: 140 - protein, 10 aa, Artificial Sequence
  source: /note=“Description of Artificial Sequence Synthetic peptide”
NIDPYSGGST

SEQ ID NO: 141 - protein, 5 aa, Artificial Sequence
  source: /note=“Description of Artificial Sequence Synthetic peptide”
GYNMN

SEQ ID NO: 142 - protein, 6 aa, Artificial Sequence
  source: /note=“Description of Artificial Sequence Synthetic peptide”
TGYNMN

SEQ ID NO: 143 - protein, 13 aa, Artificial Sequence
  source: /note=“Description of Artificial Sequence Synthetic peptide”
WIGNIDPYSGGST

SEQ ID NO: 144 - protein, 11 aa, Artificial Sequence
  source: /note=“Description of Artificial Sequence Synthetic peptide”
ARSVYDAPWLA

SEQ ID NO: 145 - protein, 7 aa, Artificial Sequence
  source: /note=“Description of Artificial Sequence Synthetic peptide”
YIYLAWY

SEQ ID NO: 146 - protein, 10 aa, Artificial Sequence
  source: /note=“Description of Artificial Sequence Synthetic peptide”
LLVYNAKTLA

SEQ ID NO: 147 - protein, 8 aa, Artificial Sequence
  source: /note=“Description of Artificial Sequence Synthetic peptide”
QHHYGTPP

SEQ ID NO: 148 - protein, 11 aa, Artificial Sequence
  source: /note=“Description of Artificial Sequence Synthetic peptide”
ARSFYDAPYLT

SEQ ID NO: 149 - protein, 7 aa, Artificial Sequence
  source: /note=“Description of Artificial Sequence Synthetic peptide”
FIYLAWY

SEQ ID NO: 150 - protein, 10 aa, Artificial Sequence
  source: /note=“Description of Artificial Sequence Synthetic peptide”
LLVFNAKTLA

SEQ ID NO: 151 - protein, 7 aa, Artificial Sequence
  source: /note=“Description of Artificial Sequence Synthetic peptide”
GYTFTSF

SEQ ID NO: 152 - protein, 6 aa, Artificial Sequence
  source: /note=“Description of Artificial Sequence Synthetic peptide”
IPNTDY

SEQ ID NO: 153 - protein, 10 aa, Artificial Sequence
  source: /note=“Description of Artificial Sequence Synthetic peptide”
YIIPNTDYTE

SEQ ID NO: 154 - protein, 5 aa, Artificial Sequence
  source: /note=“Description of Artificial Sequence Synthetic peptide”
SFWIH

SEQ ID NO: 155 - protein, 6 aa, Artificial Sequence
  source: /note=“Description of Artificial Sequence Synthetic peptide”
TSFWIH

SEQ ID NO: 156 - protein, 13 aa, Artificial Sequence
  source: /note=“Description of Artificial Sequence Synthetic peptide”
WIGYIIPNTDYTE

SEQ ID NO: 157 - protein, 11 aa, Artificial Sequence
  source: /note=“Description of Artificial Sequence Synthetic peptide”
ARGLRGAYYFD

SEQ ID NO: 158 - protein, 11 aa, Artificial Sequence
  source: /note=“Description of Artificial Sequence Synthetic peptide”
STSTNGYMHWY

SEQ ID NO: 159 - protein, 10 aa, Artificial Sequence
  source: /note=“Description of Artificial Sequence Synthetic peptide”
LLIMYASNLE

SEQ ID NO: 160 - protein, 8 aa, Artificial Sequence
  source: /note=“Description of Artificial Sequence Synthetic peptide”
QHSWVLPY

SEQ ID NO: 161 - protein, 6 aa, Artificial Sequence
  source: /note=“Description of Artificial Sequence Synthetic 6xHis tag”
HHHHHH